SEQUENCE LISTING (30 sequences)

SEQ ID NO: 1 - protein, 118 aa, Synthetic construct
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSS

SEQ ID NO: 2 - protein, 112 aa, Synthetic construct
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIK

SEQ ID NO: 3 - protein, 118 aa, Synthetic construct
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSS

SEQ ID NO: 4 - protein, 118 aa, Synthetic construct
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCRTYFYSFSYWGQGTLVTVSS

SEQ ID NO: 5 - protein, 6 aa, Synthetic construct
ASTKGP

SEQ ID NO: 6 - protein, 12 aa, Synthetic construct
TVAAPSVFIFPP

SEQ ID NO: 7 - protein, 19 aa, Synthetic construct
MDWTWRILFLVAAATGAHS

SEQ ID NO: 8 - protein, 347 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 9 - protein, 574 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 10 - protein, 332 aa, Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 347 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCRTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 12 - protein, 574 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCRTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 13 - protein, 347 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 14 - protein, 574 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 15 - protein, 24 aa, Synthetic construct
MPMGSLQPLATLYLLGMLVASVLA

SEQ ID NO: 16 - protein, 118 aa, Synthetic construct
EVKLVESGGGLVQPGGTMKLSCEISGLTFRNYWMSWVRQSPEKGLEWVAEIRLRSDNYATHYAESVKGKFTISRDDSKSRLYLQMNSLRTEDTGIYYCKTYFYSFSYWGQGTLVTVSA

SEQ ID NO: 17 - protein, 112 aa, Synthetic construct
DVVMTQTPLSLPVRLGDQASISCRSSQSLLHTYGSPYLNWYLQKPGQSPKLLIYKVSNRFSGVPDRFSGSGSGTDFTLRISRVEAEDLGVYFCSQGTHLPYTFGGGTKLEIK

SEQ ID NO: 18 - protein, 5 aa, Synthetic construct
NYWMS

SEQ ID NO: 19 - protein, 19 aa, Synthetic construct
EIRLRSDNYATHYAESVKG

SEQ ID NO: 20 - protein, 7 aa, Synthetic construct
YFYSFSY

SEQ ID NO: 21 - protein, 16 aa, Synthetic construct
RSSQSLLHTYGSPYLN

SEQ ID NO: 22 - protein, 7 aa, Synthetic construct
KVSNRFS

SEQ ID NO: 23 - protein, 9 aa, Synthetic construct
SQGTHLPYT

SEQ ID NO: 24 - protein, 471 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 25 - protein, 698 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 26 - protein, 471 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 27 - protein, 698 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 28 - protein, 471 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCRTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 29 - protein, 698 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCCTYFYSFSYWGQGTLVTVSSASTKGPEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCRTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 30 - protein, 450 aa, Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKASTKGPELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKASTKGPELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0312472 A1
(43) Pub. Date: Oct. 9, 2025

(54) ANTIBODY COMPOUNDS WITH REACTIVE CYSTEINE AND RELATED ANTIBODY DRUG CONJUGATES

(71) Applicant: UNIVERSITY OF FLORIDA RESEARCH FOUNDATION, INCORPORATED, Gainesville, FL (US)

(72) Inventors: Dobeen HWANG, Palm Beach Gardens, FL (US); Christoph RADER, Jupiter, FL (US)

(21) Appl. No.: 18/860,551

(22) PCT Filed: Apr. 28, 2023
(86) PCT No.: PCT/US2023/066399

§ 371 Date: Oct. 25, 2024

Related U.S. Application Data
(60) Provisional application No. 63/336,830, filed on Apr. 29, 2022.

(51) Int. Cl. A61K 47/68; A61P 35/00; C07K 16/32

(52) U.S. Cl. CPC A61K 47/68031; A61K 47/6879; A61K 47/6889; A61P 35/00; C07K 16/32; C07K 2317/24; C07K 2317/31; C07K 2317/55; C07K 2317/565

(57) ABSTRACT

The present invention provides antibody compounds that contain a substitution of cysteine for the reactive lysine residue (lysine 93 by Kabat numbering) in the hydrophobic cleft (38C2_Cys). The invention also provides antibody drug conjugate compounds (ADCs) that contain cargo moieties that are site-specifically conjugated to the engineered cysteine residue in the 38C2_Cys variant antibody. Further provided in the invention are therapeutic applications of the compounds.

### CROSS REFERENCE TO RELATED APPLICATION

This application claims the benefit of U.S. 63/336,830 filed Apr. 29, 2022, which is incorporated by reference in its entirety for all purposes.

### FEDERALLY SPONSORED RESEARCH OR DEVELOPMENT

This invention was made with government support under Grant Numbers R01 CA174844, R01 CA181258, and R01 CA204484 awarded by The National Institutes of Health. The government has certain rights in the invention.

### REFERENCE TO A SEQUENCE LISTING

The Sequence Listing written in file 591926SEQLST.xml is 34,535 bytes, was created Mar. 30, 2023, and is hereby incorporated by reference.

### BACKGROUND OF THE INVENTION

Antibody drug conjugates (ADCs) include three components cooperatively designed to improve the therapeutic index by selectively delivering drugs to cancer cells; a cancer cell-targeting monoclonal antibody (mAb) backbone, a linker, and a potent cytotoxic payload [2]. The concept of ADCs is the targeted delivery of a highly cytotoxic drug for selective (as opposed to systemic) chemotherapy, resulting in higher activity against malignant cells and lower toxicity toward healthy cells and tissues. The field has made great progress with the Food and Drug Administration (FDA) approvals of brentuximab vedotin (Adcetris®) for treating Hodgkin lymphoma and anaplastic large cell lymphoma, trastuzumab emtansine (Kadcyla®) for HER2 positive breast cancer, inotuzumab ozogamicin (Besponsa®) for acute lymphoblastic leukemia, and gemtuzumab ozogamicin (Mylotarg®) for acute myeloid leukemia. By 2022, 11 ADCs have been approved by the U.S. Food and Drug Administration (FDA), and the growing number of ADCs in clinical trials for a wide range of solid and hematologic malignancies reflects the increasing importance of this class of antibody therapeutics in oncology [1]. Of the 11 FDA-approved ADCs, 9 use an IgG1 and 2 an IgG4 format as their mAb backbone and all use either natural lysine (3) or cysteine (8) residues for linker-drug attachment due to their surface accessibility and the nucleophilicity of their &-amino and thiol group, respectively [1].

The growing pipeline of ADCs also faces challenges. While the therapeutic index, i.e. the ratio of maximum tolerated dose and minimum effective dose, is generally higher for ADCs compared to chemotherapy (Panowski et al., MAbs 6:34-45, 2014), ADCs have also encountered on-target and off-target toxicities (Donaghy, MAbs 8:659-671, 2016). The 11 FDA-approved ADCs and the vast majority of ADCs in the clinical and preclinical pipeline randomly conjugate the drug to either surface lysine (Lys) or hinge cysteine (Cys) residues of the antibody, yielding complex mixtures of molecular species with varying drug-to-antibody ratios (DARs), pharmacokinetics, and pharmacodynamics.

The ADC field is moving toward homogeneous ADCs that, ideally, consist of a single molecular species with defined pharmacological properties. Homogeneous ADCs are highly defined compositions of mAb, linker, and drug, and typically have DARs of 2 or 4. Among various site-specific conjugation strategies including engineering cysteine residues (e.g., Junutula et al., Nat. Biotechnol. 26:925, 2008), unnatural amino acids (e.g., Axup et al., Proc. Natl. Acad. Sci. U.S.A. 109:16101, 2012), and enzymatic conjugation (e.g., Strop et al., Chem. Biol. 20:161, 2013), the chemically programmable catalytic antibody (h38C2) has been utilized to generate a site-specific ADC in one-step conjugation as reported in Nanna et al., Nat. Commun. 8:1112, 2017, and [8]. h38C2 is a humanized anti-hapten antibody binding to 1,3-diketone or β-lactam at a uniquely nucleophilic reactive lysine (K99) at the bottom of a hydrophobic pocket. The amino acid residues lining the hydrophobic pocket surround the reactive lysine residue and contribute to its unusual low pKa of 6.3 (Barbas et al., Science 278:2085, 1997). K99 of the heavy chain is located at the bottom of a 11-Å deep pocket that permits site-specific conjugation of β-diketone-, β-lactam-, and heteroaryl methylsulfonyl-functionalized payloads [9-12]. When combined with a targeting antibody in a dual variable domain (DVD) format, h38C2-based ADCs revealed high homogeneity, stability, and potency in vitro and in vivo [12, 13]. Replacing the reactive lysine with an arginine (K99R) afforded an orthogonal site-specific conjugation of phenylglyoxal-functionalized payloads and an opportunity to assemble dual payload ADCs in a one-pot reaction when combining reactive lysine and arginine in one antibody [14]. Unlike the lysine conjugation, arginine conjugation is not complete due to partial blockade of the reactive arginine with the cellular metabolite methylglyoxal [15].

Nevertheless, there is still a need in the art for a more efficient generation of ADCs with single and multiple payloads in a chemically defined manner. The instant invention is directed to this and other unmet needs.

### SUMMARY OF THE INVENTION

In one aspect, the invention provides an antibody compound comprising a binding site comprising a heavy chain variable region comprising CDRs of SEQ ID NO:1 and a light chain variable region comprising CDRs of SEQ ID NO:2, wherein position 93 of the heavy chain variable region by Kabat numbering is occupied by cysteine. Some antibody compounds are humanized. In some antibody compounds, the heavy chain and light chain variable regions comprise SEQ ID NOs: 1 and 2 respectively.

Some antibody compounds of the invention are dual variable domain (DVD) compounds comprising (i) the binding site, and (ii) a second binding site comprising a heavy chain variable region and a light chain variable region recognizing a target of interest. In some antibody compounds, heavy and light chain variable regions of the second binding site are linked to N-termini of the heavy and light chain variable regions of the binding site.

Some antibody compounds are homodimeric molecules comprising two antibody arms, each comprising the binding site and the second binding site.

Some antibody compounds are heterodimeric molecules comprising two arms, one arm comprising the binding site and the second binding site, the other arm comprising the binding site with lysine instead of cysteine at position 93 of the heavy chain variable region by Kabat numbering and the second binding site. Some antibody compounds are heterodimeric molecules comprising two arms, one arm comprising the binding site and the second binding site, the other arm comprising the binding site with arginine instead of cysteine at position 93 of the heavy chain variable region by Kabat numbering and the second binding site.

Some antibody compounds are heterodimeric molecules comprising two arms, one arm comprising the binding site and the binding site with arginine instead of cysteine at position 93 of the heavy chain variable region by Kabat numbering, the other arm comprising the binding site with lysine instead of cysteine at position 93 of the heavy chain variable region by Kabat numbering and the second binding site. Some antibody compounds are heterodimeric molecules comprising two arms, one arm comprising the binding site and the binding site with lysine instead of cysteine at position 93 of the heavy chain variable region by Kabat numbering, the other arm comprising the binding site with arginine instead of cysteine at position 93 of the heavy chain variable region by Kabat numbering and the second binding site. Some antibody compounds are heterodimeric molecules comprising two arms, one arm comprising the binding site with lysine instead of cysteine at position 93 of the heavy chain variable region by Kabat numbering and the binding site with arginine instead of cysteine at position 93 of the heavy chain variable region by Kabat numbering, the other arm comprising the binding site and the second binding site.

Some antibody compounds of the invention are triple variable domain (TVD) compounds comprising (i) the binding site, (ii) the binding site, the binding site with arginine instead of cysteine at position 93 of the heavy chain variable region by Kabat numbering, or the binding site with lysine instead of cysteine at position 93 of the heavy chain variable region by Kabat numbering and (iii) a second binding site comprising a heavy chain variable region and a light chain variable region recognizing a target of interest.

In some antibody compounds, heavy and light chain variable regions of the second binding site are linked to N-termini of the heavy and light chain variable regions of the binding site. Some antibody compounds are heterodimeric molecules comprising two arms, one arm comprising the binding site, the binding site with arginine instead of cysteine at position 93 of the heavy chain variable region by Kabat numbering, and the second binding site, the other arm comprising the binding site, the binding site with lysine instead of cysteine at position 93 of the heavy chain variable region by Kabat numbering, and the second binding site.

In some antibody compounds, the dual variable domain compound is a bispecific immunoglobulin molecule. In some antibody compounds, the binding site is a Fab, Fab′, F(ab′)₂, Fv or scFv.

In some antibody compounds, the target of interest is different than the target recognized by the binding site, wherein the triple variable domain compound is a bispecific immunoglobulin molecule. In some antibody compounds, the binding site is a Fab, Fab′, F(ab′)₂, Fv or scFv. In some antibody compounds, the binding site is a Fab.

In some antibody compounds, the binding site or second binding site or both comprises a humanized immunoglobulin sequence.

In some antibody compounds, the target of interest is a tumor cell surface antigen. In some antibody compounds, the tumor cell surface antigen is HER2, HER3, HER4, EGFR, EGFRvIII, FOLR1, FCMR (TOSO), CD19, CD22, CD30, CD33, CD123, CD138, CD79B, PSMA, BCMA, CD38, SLAMF7, Siglec-6, Siglec-15, PDL1, CD70, NECTIN4, TROP2, tissue factor, integrin avb3, GD2, ROR1 or ROR2.

In another aspect, the invention provides an antibody drug conjugate (ADC) comprising at least one drug moiety that is conjugated to an antibody compound via a reactive cysteine residue in the antibody compound, wherein the antibody compound comprise a binding site comprising a heavy chain variable region comprising CDRs of SEQ ID NO: 1 and a light chain variable region comprising CDRs of SEQ ID NO:2, wherein position 93 of the heavy chain variable region by Kabat numbering is occupied by cysteine. In some antibody drug conjugates, the antibody compound is humanized.

In some antibody drug conjugates, the antibody compound is a dual variable domain (DVD) compound comprising (i) the binding site, and (ii) a second binding site comprising a heavy chain variable region and a light chain variable region recognizing a target of interest. In some antibody drug conjugates, the DVD compound is a homodimeric molecule comprising two antibody arms, each comprising the binding site and the second binding site.

In some antibody drug conjugates, the DVD compound is a heterodimeric molecule comprising two arms, one arm comprising the binding site and the second binding site, the other arm comprising the binding site with lysine instead of cysteine at position 93 of the heavy chain variable region by Kabat numbering and the second binding site. In some antibody drug conjugates, the DVD compound is a heterodimeric molecule comprising two arms, one arm comprising the binding site and the second binding site, the other arm comprising the binding site with arginine instead of cysteine at position 93 of the heavy chain variable region by Kabat numbering and the second binding site.

In some antibody drug conjugates, the DVD compound is a heterodimeric molecule comprising two arms, one arm comprising the binding site and the binding site with arginine instead of cysteine at position 93 of the heavy chain variable region by Kabat numbering, the other arm comprising the binding site with lysine instead of cysteine at position 93 of the heavy chain variable region by Kabat numbering and the second binding site. In some antibody drug conjugates, the DVD compound is a heterodimeric molecule comprising two arms, one arm comprising the binding site and the binding site with lysine instead of cysteine at position 93 of the heavy chain variable region by Kabat numbering, the other arm comprising the binding site with arginine instead of cysteine at position 93 of the heavy chain variable region by Kabat numbering and the second binding site. In some antibody drug conjugates, the DVD compound is a heterodimeric molecule comprising two arms, one arm comprising the binding site with lysine instead of cysteine at position 93 of the heavy chain variable region by Kabat numbering and the binding site with arginine instead of cysteine at position 93 of the heavy chain variable region by Kabat numbering, the other arm comprising the binding site and the second binding site.

In some antibody drug conjugates, the antibody compound is a triple variable domain (TVD) compound comprising (i) the binding site, (ii) the binding site, the binding site with arginine instead of cysteine at position 93 of the heavy chain variable region by Kabat numbering, or the binding site with lysine instead of cysteine at position 93 of the heavy chain variable region by Kabat numbering and (iii) a second binding site comprising a heavy chain variable region and a light chain variable region recognizing a target of interest. Some antibody drug conjugates are heterodimeric molecules comprising two arms, one arm comprising the binding site, the binding site with arginine instead of cysteine at position 93 of the heavy chain variable region by Kabat numbering, and the second binding site, the other arm comprising the binding site, the binding site with lysine instead of cysteine at position 93 of the heavy chain variable region by Kabat numbering, and the second binding site.

In some antibody drug conjugates, the drug moiety is conjugated to the antibody compound via a linker moiety. In some antibody drug conjugates, the drug moiety is derivatized with the linker moiety prior to conjugation with the antibody compound. In some antibody drug conjugates, the linker moiety is a cleavable linker. In some antibody drug conjugates, the linker moiety comprises maleimide, monobromomaleimide, or dibromomaleimide.

In some antibody drug conjugates, the antibody compound comprises an antigen/hapten-binding fragment of a dual variable domain (DVD) compound that is a Fab, Fab′, F(ab′)₂, Fv or scFv. In some antibody drug conjugates, the antibody compound comprises a Fab.

In some antibody drug conjugates, the antibody compound comprises an antigen/hapten-binding fragment of a triple variable domain (TVD) compound that is a Fab, Fab′, F(ab′)₂, Fv or scFv. In some antibody drug conjugates, the antibody compound comprises a Fab.

In some antibody drug conjugates, the target of interest is a tumor cell surface antigen. In some antibody drug conjugates, the tumor cell surface antigen is HER2, HER3, HER4, EGFR, EGFRvIII, FOLR1, FCMR (TOSO), CD19, CD22, CD30, CD33, CD123, CD138, CD79B, PSMA, BCMA, CD38, SLAMF7, Siglec-6, Siglec-15, PDL1, CD70, NECTIN4, TROP2, tissue factor, integrin avb3, GD2, ROR1 or ROR2.

In some antibody drug conjugates, the drug moiety is a cytotoxic agent, an siRNA, or a small molecule-based proteolysis targeting chimera. In some antibody drug conjugates, the cytotoxic agent is selected from a toxin, a chemotherapeutic agent, a photoabsorber, an antibiotic, a radioactive isotope, a chelated radioactive isotope and a nucleolytic enzyme.

In some antibody drug conjugates, the binding site comprises heavy chain and light chain variable domain sequences respectively shown in SEQ ID NOs: 1 and 2, and the target of interest is HER2.

In some antibody drug conjugates, the drug moiety is an auristatin, a dolostatin, a cemadotin, a camptothecin, an amanitin, a maytansinoid, a pyrrolobenzodiazepine, an indolinobenzodiazepine, a duocarmycin, an endiyne, a doxorubicin, a cepafungin or a Fleximer. In some antibody drug conjugates, the drug moiety is monomethyl auristatin F (MMAF).

In some antibody drug conjugates, the antibody compound is a DVD-Fab comprising heavy chain and light chain sequences shown in SEQ ID NOs: 8 and 10, respectively. In some antibody drug conjugates, the antibody compound is a DVD-IgG1 comprising heavy chain and light chain sequences shown in SEQ ID NOs: 9 and 10, respectively.

In some antibody drug conjugates, the DVD-IgG1 is a homodimeric molecule comprising two antibody arms, each comprising heavy chain and light chain sequences shown in SEQ ID NOs: 9 and 10, respectively.

In some antibody drug conjugates, the DVD-IgG1 is a heterodimeric molecule comprising two arms, one arm comprising heavy chain and light chain sequences shown in SEQ ID NOs: 9 and 10, respectively, the other arm comprising heavy chain and light chain sequences shown in SEQ ID NOs: 14 and 10, respectively. In some antibody drug conjugates, the DVD-IgG1 is a heterodimeric molecule comprising two arms, one arm comprising heavy chain and light chain sequences shown in SEQ ID NOs: 9 and 10, respectively, the other arm comprising heavy chain and light chain sequences shown in SEQ ID NOs: 12 and 10, respectively. In some antibody drug conjugates, two different drug moieties are conjugated to the two antibody arms of the heterodimeric DVD-IgG1 molecule.

In some antibody drug conjugates, the antibody compound is a TVD-Fab comprising a heavy chain as shown in any of SEQ ID NOs: 24, 26, or 28 and a light chain sequence as shown in SEQ ID NO:30. In some antibody drug conjugates, the antibody compound is a TVD-IgG1 comprising a heavy chain as shown in any of SEQ ID NOs: 25, 27, or 29 and a light chain sequence as shown in SEQ ID NO:30.

In some antibody drug conjugates, the TVD-IgG1 is a homodimeric molecule comprising two antibody arms, each comprising a heavy chain as shown in SEQ ID NO:25 and a light chain sequence as shown in SEQ ID NO:30. In some antibody drug conjugates, the TVD-IgG1 is a homodimeric molecule comprising two antibody arms, each comprising a heavy chain as shown in SEQ ID NO:27 and a light chain sequence as shown in SEQ ID NO: 30. In some antibody drug conjugates, the TVD-IgG1 is a homodimeric molecule comprising two antibody arms, each comprising a heavy chain as shown in SEQ ID NO:29 and a light chain sequence as shown in SEQ ID NO:30.

In some antibody drug conjugates, the TVD-IgG1 is a heterodimeric molecule comprising two arms, one arm comprising heavy chain and light chain sequences shown in SEQ ID NOs: 25 and 30, respectively, the other arm comprising heavy chain and light chain sequences shown in SEQ ID NOs: 27 and 30, respectively. In some antibody drug conjugates, the TVD-IgG1 is a heterodimeric molecule comprising two arms, one arm comprising heavy chain and light chain sequences shown in SEQ ID NOs: 25 and 30, respectively, the other arm comprising heavy chain and light chain sequences shown in SEQ ID NOs: 29 and 30, respectively. In some antibody drug conjugates, the TVD-IgG1 is a heterodimeric molecule comprising two arms, one arm comprising heavy chain and light chain sequences shown in SEQ ID NOs: 27 and 30, respectively, the other arm comprising heavy chain and light chain sequences shown in SEQ ID NOs: 29 and 30, respectively.

In some antibody drug conjugates, two different drug moieties are conjugated to the two antibody arms of the heterodimeric TVD-IgG1 molecule. In some antibody drug conjugates, three different drug moieties are conjugated to the antibody arms of the heterodimeric TVD-IgG1 molecule.

In some antibody drug conjugates, a first drug moiety is conjugated to the binding site, a second drug moiety is conjugated to the binding site with arginine instead of cysteine at position 93 of the heavy chain variable region by Kabat numbering, and a third drug moiety is conjugated to the binding site with lysine instead of cysteine at position 93 of the heavy chain variable region by Kabat numbering. In some antibody drug conjugates, a first drug moiety is conjugated to the binding site, a second drug moiety is conjugated to the binding site with arginine instead of cysteine at position 93 of the heavy chain variable region by Kabat numbering, and a third drug moiety is conjugated to the binding site with lysine instead of cysteine at position 93 of the heavy chain variable region by Kabat numbering, wherein the first, second, and third drug moieties are different from each other. In some antibody drug conjugates, a first drug moiety is conjugated to the binding site and a second drug moiety is conjugated to the binding site with arginine instead of cysteine at position 93 of the heavy chain variable region by Kabat numbering, or the binding site with lysine instead of cysteine at position 93 of the heavy chain variable region by Kabat numbering, wherein the first and second drug moieties are different from each other.

In another aspect, the invention provides a pharmaceutical composition, comprising an effective amount of any of the antibody drug conjugates disclosed herein and optionally a pharmaceutically acceptable carrier. In another aspect the invention provides a method for treating cancer in a subject, comprising administering to the subject in need of treatment any of the pharmaceutical compositions described herein.

A further understanding of the nature and advantages of the present invention may be realized by reference to the remaining portions of the specification and claims.

### DESCRIPTION OF THE DRAWINGS

FIGS. 1A-E. Site specific conjugation of h38C2_K99C (A) The DVD-Fab is composed of variable domains of trastuzumab (outer Fv) and h38C2_K99C mutant (inner Fv) and constant domain. (B) DVD-Fab and Fab revealed the expected 70- and 35-kDa bands under nonreducing (NR) conditions, and the expected 50- and 25-kDa bands under reducing (R) conditions after Coomassie Blue staining. (C) The catalytic activity of parental (h38C2_Lys) and mutated (h28C2_Cys; h38C2_K99C) was measured using the retro-aldol reaction of methodol to detect a fluorescent aldehyde (RFU, relative fluorescent units). Mean (±) SD values of triplicates were plotted. (D) DVD-Fabs with h38C2_K99C, h38C2_K99Y, and h38C2_K99 were incubated with 50 μM maleimide derivatives of TAMRA (compounds 1, 2, 3; FIG. 3). Conjugates were subjected to SDS-PAGE and analyzed by fluorescent imaging and Coomassie Blue staining. (E) Covalent conjugation of a dibromomaleimide derivative to the K99C residue of h38C2.

FIG. 2. Crystal structure of h38C2_K99C Fab projected onto an IgG1 molecule. The K99C mutation and the intradomain disulfide bridge formed by C22 and C98 of h38C2 were identified by X-ray crystallography (PDB ID: 7TUS). The variable heavy chain (VH) is shown as shaded.

FIG. 3. Structure of maleimide derivatives.

FIG. 4. Human plasma stability of anti-HER2 h38C2_K99C_3 DVD-Fab. The conjugate was incubated in human plasma at 37° C. for up to 8 days. Aliquots at each time point were analyzed by Coomassie Blue staining (top) and in-gel fluorescence (bottom).

FIGS. 5A-B. Docking simulations of K99C and dibromomaleimide ligand. (A) The bulk of the stabilization of the ligand is provided by the aromatic side chains from the binding pocket. (B) Schematic visualization of the covalent thio-monobromomaleimide interactions.

FIGS. 6A-B. Characterization of ADCs based on h38C2_K99C. (A) ESI-TOF analysis of compound 4 conjugated anti-HER2 DVD-Fab K99C. (B) Cell-based cytotoxicity of anti-HER2 DVD-Fab and DVD-IgG ADCs after conjugation to β-lactam-hapten-MMAF or dibromomaleimide-MMAF (compound 4) following incubation with HER2+SK-BR-3 and HER2-MDA-MB-231 cells for 72 h at 37 C. Mean (±) SD values of triplicates were plotted.

FIG. 7. Analysis of the conjugation efficiency of maleimide derivatives 1, 2, and 3 to the h38C2_K99C-based anti-HER2 DVD-Fab and MS-PODA derivative 5 to the parental h38C2-based DVD-Fab by ESI-TOF.

FIG. 8. Comparison of antigen binding of anti-HER2 DVD-Fab h38C2_K99C before and after the conjugation to compound 3 by SPR. Five different concentrations (12.5, 25, 50, 100, and 200 nM) of DVD-Fabs were injected to measure the affinity of DVD-Fabs to HER2-Fc captured by an anti-human Fcγ mAb immobilized on a CM5 chip.

FIG. 9. Overlay of crystal structures of the hydrophobic pocket of h38C2_K99R (hatched with checkerboard pattern) and h38C2_K99C (hatched with lines angled to left). Structurally divergent regions are hatched, structurally conserved regions shown as unfilled. Key residues are shown in sticks. Residues marked in bold text are in h38C2_K99R. Residues marked in italicized text are in h38C2_K99C. OH— (hydroxy) groups are hatched with interlocking rectangle pattern. NH₂-(amine) groups are hatched with dotted pattern.

FIGS. 10A-B. (A) Representative bound states of the docked ligands showing the preferential binding of the compound 3-mimicking ‘DiBromo’ ligand at the K99C site in two rotationally isomeric orientations, while the compound 1-mimicking ‘NoBromo’ ligand solvated away from the conjugation site in all docking runs. (B) Similar contributions from restraint energies across all the docked models suggest no artifactual energy barriers were created between the bound conformations compared to the unbound ones. In FIG. 10A, DiBromo-1 docking simulation hatched with lines angled to left and dotted, DiBromo-2 docking simulation hatched with lines angled to right, and NoBromo-2 docking simulation hatched with dotted pattern. In FIG. 10A, Oxygen (O═) groups are hatched with checkerboard pattern, nitrogen (N—) groups are solid filled, and bromine (Br—) groups are unfilled.

FIGS. 11A, 11B, 11C, 11D, 11E, 11E continued, 11F, 11G, 11G, continued, 11H, 11H continued, 11I, 11I continued, 11J, 11J continued, 11K, 11K continued, 11L, and 11L continued. Analytical data (LC-MS) of compounds for bioconjugation.

FIGS. 12A-12C. Orthogonal conjugation of TVD-Fab (Cys_Lys) (A) TVD-Fab (Cys_Lys) is composed of variable domains of anti-HER2 trastuzumab (outer Fv), h38C2_K99C (upper inner Fv), h38C2 (lower inner Fv), and constant domains. (B) TVD-Fab (Cys_Lys) revealed the expected ˜100-kDa band under nonreducing conditions by Coomassie blue staining. To assemble an antibody-fluorophore conjugate, 10 μg of TVD-Fab (Cys_Lys) was incubated with 5 eq of dibromomaleimide-TAMRA or β-lactam-TAMRA, respectively, for 1 h and run on a 4-12% SDS-PAGE gel. Blue light visualization revealed a strong fluorescent band of ˜100-kDa. (C) The catalytic activity of TVD-Fab (Cys_Lys) before and after conjugation to β-lactam-TAMRA was measured using the retro-aldol reaction of methodol to detect a fluorescent aldehyde (RFU, relative fluorescent units). Mean (±) SD values of triplicates were plotted.

### DETAILED DESCRIPTION

### I. Overview

The present invention is directed to ADCs that involve site-specific cysteine conjugation of drug moieties to an antibody compound that contains a variant of catalytic antibody 38C2. Cysteine is a particularly useful amino acid for protein modification due to the thiol moiety having the highest nucleophilicity of all functional groups of proteinogenic amino acid under physiological conditions [3]. Utilizing the four interchain disulfide bridges of the IgG1 hinge region is favored for bioconjugation as it affords rapid and efficient ADC assembly through reduction of interchain disulfide bridges followed by thiol-maleimide reaction to attach the payload. However, cysteine bioconjugation is inherently prone to generate a heterogenous mixture of bioconjugates with drug-to-antibody ratios (DARs) ranging from 0 to 8 [4]. The mixture is comprised of numerous species with different pharmacokinetic and pharmacodynamic properties [5]. Moreover, the thiol-maleimide adduct, i.e. the thiosuccinimide linkage of ADCs, is prone to a retro-Michael reaction-triggered payload loss in the blood [6, 7]. Extensive efforts have been directed toward both natural and engineered cysteine-based ADC assembly strategies that overcome the shortcomings of first-generation ADCs to afford homogenous and stable bioconjugates [4].

The present invention is predicated in part on the studies undertaken by the inventors to generate a distinctive environment for site-specific conjugation to a cysteine residue inside the hydrophobic pocket of h38C2. A K99R mutant of h38C2 that replaces the reactive lysine with an arginine is known in the art, where the introduced arginine residue in h38C2_Arg has unique reactivity that permits its selective and stable conjugation to phenylglyoxal derivatives. (WO 2020/076849).

Pursuing a conceptually similar strategy with improved conjugation efficiency, the inventors here describe the generation and utilization of a K99C mutant of h38C2 that replaces the reactive lysine with a cysteine. Unexpectedly, dibromomaleimide-functionalized payloads, usually employed to bridge two cysteines [16], emerged as best match for K99C when analyzed biochemically and structurally. Collectively, we introduce a new engineered cysteine-based site-specific ADC assembly strategy that offers precise, fast, efficient, and stable payload attachment.

Here we expand the scope of this ADC assembly strategy by mutating h38C2's reactive lysine to a cysteine. X-ray crystallography of this point mutant, h38C2_K99C, confirmed a deeply buried unpaired cysteine. Probing h38C2_K99C with maleimide, monobromomaleimide, and dibromomaleimide derivatives of a fluorophore revealed highly disparate conjugation efficiencies and stabilities. Dibromomaleimide emerged as a suitable electrophile for precise, fast, efficient, and stable assembly of ADCs with the h38C2_K99C module. Mass spectrometry indicated the presence of a thio-monobromomaleimide linkage which was further supported by in silico docking studies. Using a dibromomaleimide derivative of the highly potent tubulin polymerization inhibitor monomethyl auristatin F (MMAF), h38C2_K99C-based ADCs were found to be as potent as h38C2-based ADCs and afford a new assembly route for ADCs with single and dual payloads.

In accordance with these exemplified studies, the present invention provides novel site-specific cysteine conjugated antibody drugs and related uses. As described herein, the ADCs of the invention contain at least one drug moiety that is conjugated to an antibody compound via a reactive cysteine residue in the antibody compound. Preferably, the antibody compound in the ADCs of the invention contains a variant of catalytic antibody 38C2, or hapten binding fragment thereof, that contains a substitution of cysteine for the reactive lysine residue in the hydrophobic cleft (38C2_Cys). In various embodiments, the antibody compound of the ADCs is a dual variable domain (DVD) compound or an antigen/hapten-binding fragment thereof that contains (i) the 38C2_Cys or hapten binding fragment thereof, and (ii) a second antibody variable domain recognizing a target of interest. In other embodiments, the antibody compound of the ADC is a triple variable domain (TVD) compound or an antigen/hapten-binding fragment thereof comprising (i) a first antibody variable domain comprising a first 38C2_Cys or hapten binding fragment thereof, (ii) a second antibody variable domain comprising a 38C2_Arg, a 38C2_Lys, or a second 38C2_Cys or hapten binding fragment thereof and (iii) a third antibody variable domain recognizing a target of interest.

Before the present invention is described in greater detail, it is to be understood that this invention is not limited to particular aspects described, as such may, of course, vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

The practice of the present invention can employ, unless otherwise indicated, conventional techniques of molecular biology (including recombinant techniques), microbiology, cell biology, biochemistry, and immunology, which are within the skill of the art. Such techniques are explained fully in the literature. See, e.g., Methods in Enzymology, Volume 289: Solid-Phase Peptide Synthesis, J. N. Abelson, M. I. Simon, G. B. Fields (Editors), Academic Press; 1st edition (1997) (ISBN-13:978-0121821906); U.S. Pat. Nos. 4,965,343, and 5,849,954; Sambrook et al., Molecular Cloning: A Laboratory Manual, Cold Spring Harbor Press, N.Y., (3^{rd} ed., 2000); Brent et al., Current Protocols in Molecular Biology, John Wiley & Sons, Inc. (ringbou ed., 2003); Barbas et al., Phage Display: A Laboratory Manual, CSHL Press (2004); Davis et al., Basic Methods in Molecular Biology, Elsevier Science Publishing, Inc., New York, USA (1986); Methods in Enzymology: Guide to Molecular Cloning Techniques, Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.); Current Protocols in Cell Biology (CPCB) (Juan S. Bonifacino et. al. ed., John Wiley and Sons, Inc.); Culture of Animal Cells: A Manual of Basic Technique and Specialized Applications, R. Ian Freshney, Wiley Blackwell (7th edition, 2015); and Animal Cell Culture Methods, Jennie P. Mather and David Barnes editors, Academic Press (1^{st} edition, 1998). The following sections provide additional guidance for practicing the compositions and methods of the present invention.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible. Although any methods and materials similar or equivalent to those described herein can also be used in the practice or testing of the present invention, representative illustrative methods and materials are now described.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The basic antibody or immunoglobulin structural unit is a tetramer of subunits including two light (L) chains and two heavy (H) chains, antigen/hapten-binding fragments thereof, and complexes formed from multiple such entities. In a native antibody format the two light chains are the same and the two heavy chains are the same, and the antibody has two identical binding sites. In a bispecific antibody, either the two light chains are different from one another or the two heavy chains are different from one another or both, and the bispecific antibody has two different binding sites (see, e.g., Songsivilai and Lachmann, Clin. Exp. Immunol., 79:315-321 (1990); Kostelny et al., J. Immunol., 148:1547-53). In a native antibody, each L chain is linked to an H chain by one covalent disulfide bond, while the two H chains are linked to each other by one or more disulfide bonds depending on the H chain isotype. Each H and L chain has an N-terminus and a C-terminus, and also has regularly spaced intrachain disulfide bridges. Each H chain has at the N-terminus a variable domain (V_{H}) followed by three constant domains (C_{H}1, C_{H}2 and C_{H}3). Each L chain has at the N-terminus a variable domain (V_{L}) followed by one constant domain (C_{L}). The V_{L} is aligned with the V_{H} and the C_{L} is aligned with the first constant domain of the heavy chain (C_{H}1). Particular amino acid residues are believed to form an interface between the L chain and H chain variable domains. The pairing of a V_{H} and V_{L} together forms a single antigen/hapten-binding site.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The “variable region” or “variable domain” of an immunoglobulin refers to the N-terminal domains of the H or L chain of the immunoglobulin. The variable domain of the H chain can be referred to as “V_{H}.” The variable domain of the light chain can be referred to as “V_{L}.” These domains are generally the most variable parts of an immunoglobulin and contain the antigen/hapten-binding sites. That is, a binding site includes a VH and VL, which can duplex intermolecularly as separate chains or intramolecularly as components of the same chain, as in an scFv.

The term “variable” refers to the fact that certain segments of the variable domains differ extensively in sequence among immunoglobulins. The V domain mediates antigen or hapten binding and defines specificity of a particular immunoglobulin for its particular antigen or hapten. However, the variability is not evenly distributed across the 110-amino acid span of most variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native H and L chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen/hapten-binding site of immunoglobulins (see Kabat et al., Sequences of Proteins of Immunological Interest, 5th Ed. Public Health Service, National Institutes of Health, Bethesda, MD. (1991)). The constant domains are not involved directly in binding an immunoglobulin to an antigen or hapten, but exhibit various effector functions, such as participation of the immunoglobulin in antibody dependent cellular cytotoxicity (ADCC), antibody-dependent cellular phagocytosis (ADCP), and complement-dependent cytotoxicity (CDC).

An “intact” immunoglobulin is one that comprises an antigen/hapten-binding site as well as a C_{L} and at least H chain constant domains, C_{H}1, C_{H}2 and C_{H}3. The constant domains can be native sequence constant domains (e.g., human native sequence constant domains) or amino acid sequence variants thereof. An intact immunoglobulin can have one or more effector functions.

A “naked immunoglobulin” for the purposes herein is an immunoglobulin that is not conjugated to a drug moiety.

“Immunoglobulin fragments” comprise a portion of an intact immunoglobulin, preferably the antigen or hapten binding or variable region of the intact immunoglobulin. Examples of immunoglobulin fragments include, but are not limited to, Fab, Fab′, F(ab′)₂, and Fv fragments; diabodies; linear immunoglobulins (see U.S. Pat. No. 5,641,870, Example 2; Zapata et al., Protein Eng. 8(10): 1057-1062 [1995]); single-chain immunoglobulin molecules; and multispecific immunoglobulins formed from immunoglobulin fragments. In some embodiments, the immunoglobulin fragments include all possible alternate fragment formats. In some embodiments, the immunoglobulin fragments may be bispecific. In some embodiments, the immunoglobulin fragments may be bi-paratopic. In some embodiments, the immunoglobulin fragments may be trispecific. In some embodiments, the immunoglobulin fragments may be multimeric. In some embodiments, an immunoglobulin fragment comprises an antigen or hapten binding site of the intact immunoglobulin and thus retains the ability to bind antigen or hapten. In some embodiments, the immunoglobulin fragment contains single variable domains which have the ability to bind antigen or hapten. In some embodiments, the immunoglobulin fragments are further modified (not limited to peptide addition, pegylation, hesylation, glycosylation) to modulate activity, properties, pharmacokinetic behavior and in vivo efficacy.

Fragments typically compete with the intact antibody from which they were derived from specific binding to their target. Fragments can be synthesized by recombinant techniques or by chemical or enzymatic digestions. Papain digestion of immunoglobulins produces two identical antigen/hapten-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen or hapten binding, i.e., it has a single antigen/hapten-binding site. Pepsin treatment of an immunoglobulin yields a single large F(ab′) 2 fragment which roughly corresponds to two disulfide linked Fab fragments having bivalent antigen/hapten-binding activity and is still capable of cross-linking antigen or hapten. Fab′ fragments differ from Fab fragments by having additional few residues at the carboxy terminus of the C_{H}1 domain including one or more cysteines from the immunoglobulin hinge region. Fab′-SH is the designation herein for Fab′ in which the cysteine residue(s) of the constant domains bear a free thiol group. F(ab′)₂ immunoglobulin fragments originally were produced as pairs of Fab′ fragments which have hinge cysteines between them. Other chemical couplings of immunoglobulin fragments are also known.

The Fc fragment comprises the carboxy-terminal portions of both H chains held together by disulfides. The effector functions of immunoglobulins are determined by sequences in the Fc region, which region is also the part recognized by Fc receptors (FcR) found on certain types of cells.

“Fv” is the minimum immunoglobulin fragment which contains a complete antigen/hapten recognition and -binding site. This fragment consists of a dimer of one heavy- and one light-chain variable region domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen or hapten binding and confer antigen or hapten binding specificity to the immunoglobulin. However, even a single variable domain (or half of an Fv comprising only three CDRs specific for an antigen or hapten) has the ability to recognize and bind antigen or hapten, although typically at a lower affinity than the entire binding site. When used herein in reference to a DVD or TVD immunoglobulin molecule, the term “Fv” refers to a binding fragment that includes both the first and the second variable domains of the heavy chain and the light chain.

“Single-chain Fv” also abbreviated as “sFv” or “scFv” are immunoglobulin fragments that comprise the V_{H} and V_{L} immunoglobulin domains connected into a single polypeptide chain. Preferably, the sFv polypeptide further comprises a polypeptide linker between the V_{H} and V_{L} domains which enables the sFv to form the desired structure for antigen or hapten binding. For a review of sFv, see Plückthun in The Pharmacology of Monoclonal Antibodies, vol. 113, Rosenburg and Moore eds., Springer-Verlag, New York, pp. 269-315 (1994); and Antibody Engineering, Borrebaeck ed., Oxford University Press (1995). When used herein in reference to a DVD or TVD immunoglobulin molecule, the term “scFv” refers to a binding fragment that includes both the first and the second variable domains of the heavy chain and the light chain.

As used herein, a “dual variable domain (DVD) compound” or a “dual variable domain (DVD) immunoconjugate” refers to compound that has a first and a second variable domain of immunoglobulins (including antigen/hapten-binding fragments of Ig such as Fab), and optionally a drug moiety that is covalently conjugated to a first and/or second variable domain via a linker. The term “dual variable domain immunoglobulin” or “DVD-Ig” as used herein refers to an immunoglobulin molecule the H and L chains of which both include a second variable domain located adjacent to the first variable domain. The L chain of a DVD-Ig therefore includes, from N-terminus to C-terminus, the following domains: V_{L}1-V_{L}2-C_{L}. The H chain of a DVD-Ig therefore includes, from N-terminus to C-terminus, the following domains: V_{H}1-V_{H}2-C_{H}1-C_{H}2-C_{H}3. The pairing of a V_{L}1 and V_{H}1 together forms a first antigen or hapten binding site. The pairing of a V_{L}2 and V_{H}2 together forms a second antigen or hapten binding site. A dual variable domain (DVD) IgG1 comprises an outer variable fragment (Fv) domain (outer Fv domain) and an inner Fv domain. The outer Fv domain comprises the pairing of a V_{L}1 and V_{H}1, and an inner Fv domain comprises the pairing of a V_{L}2 and V_{H}2. An outer Fv domain or an inner Fv domain may bind a target of interest. An outer Fv domain or an inner Fv domain may target tumor cells. An outer Fv domain or an inner Fv domain may comprise a reactive residue useful for site-specific conjugation of a payload. In some DVD-Ig embodiments, the outer Fv domain binds a target of interest and the inner Fv domain comprise a reactive residue useful for site-specific conjugation of a payload. In some DVD-Ig embodiments, both the outer Fv domain and the inner Fv domain comprise a reactive residue useful for site-specific conjugation of a payload. In some embodiments, the DVD compound of the invention is DVD-Fab, which contains an immunoglobulin component that is an antigen or hapten binding fragment of Ig such as a Fab fragment as exemplified herein. General methods of making various DVD compounds of the invention are described in the art, e.g., Nanna et al., Nat. Commun. 8:1112, 2017. As used herein, a “triple variable domain (TVD) compound” or a “triple variable domain (TVD) immunoconjugate” refers to compound that has a first, a second, and a third variable domain of immunoglobulins (including antigen/hapten-binding fragments of Ig such as Fab), and optionally a drug moiety that is covalently conjugated to at least one of the first, second or third variable domains via a linker. The term “triple variable domain immunoglobulin” or “TVD-Ig” as used herein refers to an immunoglobulin molecule the H and L chains of which both include a second variable domain located adjacent to the first variable domain, and a third variable domain located adjacent to the second variable domain. The L chain of a TVD-Ig therefore includes, from N-terminus to C-terminus, the following domains: V_{L}1-V_{L}2-V_{L}3-C_{L}. The H chain of a TVD-Ig therefore includes, from N-terminus to C-terminus, the following domains: V_{H}1-V_{H}2-V_{H}3-C_{H}1-C_{H}2-C_{H}3. The pairing of a V_{L}1 and V_{H}1 together forms a first antigen/hapten-binding site. The pairing of a V_{L}2 and V_{H}2 together forms a second antigen or hapten binding site. The pairing of a V_{L}3 and V_{H}3 together forms a third antigen or hapten binding site. A triple variable domain (TVD) IgG1 comprises an outer variable fragment (Fv) domain (outer Fv domain), an upper inner Fv domain, and a lower inner Fv domain. The outer Fv domain comprises the pairing of a V_{L}1 and V_{H}1, an upper inner Fv domain comprises the pairing of a V_{L}2 and V_{H}2, and a lower inner Fv domain comprises the pairing of a V_{L}3 and V_{H}3. An outer Fv domain, an upper inner Fv domain, or a lower inner Fv domain may bind a target of interest. An outer Fv domain, an upper inner Fv domain, or a lower inner Fv domain may target tumor cells. An outer Fv domain, an upper inner Fv domain, or a lower inner Fv domain may comprise a reactive residue useful for site-specific conjugation of a payload. In some TVD-Ig embodiments, the outer Fv domain binds a target of interest and the upper inner Fv domain and lower inner Fv domain each comprise a reactive residue useful for site-specific conjugation of a payload.

In some embodiments, the TVD compound of the invention is TVD-Fab, which contains an immunoglobulin component that is an antigen or hapten binding fragment of Ig such as an Fab fragment as exemplified herein. General methods of making various TVD compounds of the invention are described in the art, e.g., Hwang et al., Biomolecules 10:764, 2020.

Unless stated otherwise, the term “immunoglobulin” or “antibody” specifically includes native human and non-human IgG1, IgG2, IgG3, IgG4, IgE, IgA1, IgA2, IgD and IgM antibodies, including naturally occurring variants.

The term “native” with reference to a polypeptide (e.g., an antibody or immunoglobulin) is used herein to refer to a polypeptide having a sequence that occurs in nature, regardless of its mode of preparation. The term “non-native” with reference to a polypeptide (e.g., an antibody or immunoglobulin) is used herein to refer to a polypeptide having a sequence that does not occur in nature.

The term “polypeptide” is used herein in the broadest sense and includes peptide sequences. The term “peptide” generally describes linear molecular chains of amino acids containing up to about 30, preferably up to about 60 amino acids covalently linked by peptide bonds.

The term “monoclonal” as used herein refers to an antibody or immunoglobulin molecule (e.g., a DVD Ig molecule or a TVD Ig molecule) obtained from a population of substantially homogeneous immunoglobulins, i.e., the individual immunoglobulins comprising the population are identical except for possible naturally occurring mutations that can be present in minor amounts. Monoclonal immunoglobulins are highly specific, being directed against a single antigenic site. Furthermore, in contrast to conventional (polyclonal) antibody preparations which typically include different antibodies directed against different determinants (epitopes), each monoclonal immunoglobulin is directed against a single determinant on the antigen. The modifier “monoclonal” indicates the character of the immunoglobulin as being obtained from a substantially homogeneous population of immunoglobulins, and is not to be construed as requiring production of the antibody by any particular method. For example, the monoclonal immunoglobulins in accordance with the present invention can be made by the hybridoma method first described by Köhler and Milstein (1975) Nature 256:495, or can be made by recombinant DNA methods (see, e.g., U.S. Pat. No. 4,816,567).

The monoclonal immunoglobulins herein specifically include “chimeric” immunoglobulins in which a portion of the heavy and/or light chain is identical with or homologous to corresponding sequences in antibodies derived from a particular species, while the remainder of the chain(s) is identical with or homologous to corresponding sequences in antibodies derived from another species, as well as fragments of such antibodies, so long as they exhibit the desired biological activity (U.S. Pat. No. 4,816,567; and Morrison et al. (1984) Proc. Natl. Acad. Sci. USA 81:6851-6855).

“Humanized” forms of non-human (e.g., rodent, e.g., murine or rabbit) immunoglobulins are immunoglobulins which contain minimal sequences derived from non-human immunoglobulin. For the most part, humanized immunoglobulins are human immunoglobulins (recipient antibody) in which residues from a hypervariable region of the recipient are replaced by residues from a hypervariable region of a non-human species (donor antibody) such as mouse, rat, hamster, rabbit, chicken, bovine or non-human primate having the desired specificity, affinity, and capacity. In some instances, Fv framework region (FR) residues of the human immunoglobulin are also replaced by corresponding non-human residues. Furthermore, humanized antibodies can comprise residues which are not found in the recipient antibody or in the donor antibody. These modifications are made to further refine antibody performance. In general, the humanized immunoglobulin will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized immunoglobulin optionally also will comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. Biol. 2:593-596.

The term “human immunoglobulin”, as used herein, is intended to include immunoglobulins having variable and constant regions derived from human germline immunoglobulin sequences. The human immunoglobulins of the invention can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human immunoglobulin”, as used herein, is not intended to include immunoglobulins in which CDR sequences derived from the germline of another mammalian species, such as a mouse, have been grafted onto human framework sequences.

An “isolated” immunoglobulin herein is one which has been identified and separated and/or recovered from a component of its natural environment in a recombinant host cell. Contaminant components of its natural environment are materials which would interfere with diagnostic or therapeutic uses for the immunoglobulin, and can include enzymes, hormones, and other proteinaceous or nonproteinaceous solutes, as well as undesired byproducts of the production. In some embodiments, an isolated immunoglobulin herein will be purified (1) to greater than 95% by weight, or greater than 98% by weight, or greater than 99% by weight, as determined by SDS-PAGE or SEC-HPLC methods, (2) to a degree sufficient to obtain at least 15 residues of N-terminal or internal amino acid sequence by use of an amino acid sequencer, or (3) to homogeneity by SDS-PAGE under reducing or non-reducing conditions using Coomassie blue or, preferably, silver stain. Ordinarily, an isolated immunoglobulin will be prepared by at least one purification step.

The term “specific binding” or “specifically binds to” or is “specific for” refers to the binding of a binding moiety to a binding target, such as the binding of an immunoglobulin to a target antigen, e.g., an epitope on a particular polypeptide, peptide, or other target (e.g. a glycoprotein target), and means binding that is measurably different from a non-specific interaction (e.g., a non-specific interaction can be binding to bovine serum albumin or casein). Specific binding can be measured, for example, by determining binding of a binding moiety, or an immunoglobulin, to a target molecule compared to binding to a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a K_{d} for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain instances, the term “specific binding” refers to binding where a molecule binds to a particular polypeptide or epitope on a particular polypeptide without substantially binding to any other polypeptide or polypeptide epitope.

“Binding affinity” refers to the strength of the sum total of noncovalent interactions between a single binding site of a molecule (e.g., an immunoglobulin) and its binding partner (e.g., an antigen or hapten). Unless indicated otherwise, as used herein, “binding affinity” refers to intrinsic binding affinity which reflects a 1:1 interaction between members of a binding pair (e.g., immunoglobulin and antigen). The affinity of a molecule X for its partner Y can generally be represented by the dissociation constant (K_{d}). For example, the K_{d} can be about 200 nM, 150 nM, 100 nM, 60 nM, 50 nM, 40 nM, 30 nM, 20 nM, 10 nM, 8 nM, 6 nM, 4 nM, 2 nM, 1 nM, or stronger. Affinity can be measured by common methods known in the art, including those described herein. Low-affinity antibodies generally bind antigen or hapten slowly and tend to dissociate readily, whereas high-affinity antibodies generally bind antigen or hapten faster and tend to remain bound longer. A variety of methods of measuring binding affinity are known in the art.

As used herein, the “K_{d}” or “K_{d} value” refers to a dissociation constant measured by a technique appropriate for the immunoglobulin and target pair, for example using surface plasmon resonance assays, for example, using a Biacore X100 or a Biacore T200 (Cytiva, Piscataway, NJ) at 25° C. with immobilized antigen CM5 chips.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent association.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini. The term “fusion” is used herein to refer to the combination of amino acid sequences of different origin.

The term “epitope” includes any molecular determinant capable of specific binding to an immunoglobulin. In certain aspects, epitope determinants include chemically active surface groupings of molecules such as amino acids, sugar side chains, phosphoryl, or sulfonyl, and, in certain aspects, can have specific three dimensional structural characteristics, and/or specific charge characteristics. An epitope is a region of an antigen that is bound by an immunoglobulin. A “binding region” is a region on a binding target bound by a binding molecule.

The term “target” or “binding target” is used in the broadest sense and specifically includes polypeptides, without limitation, nucleic acids, carbohydrates, lipids, cells, and other molecules with or without biological function as they exist in nature.

The term “antigen” refers to an entity or fragment thereof, which can bind to an immunoglobulin or trigger a cellular immune response. An immunogen refers to an antigen, which can elicit an immune response in an organism, particularly an animal, more particularly a mammal including a human. The term antigen includes regions known as antigenic determinants or epitopes, as defined above.

The term “hapten” refers to an entity, for example, a small molecule, that elicits an immune response in an organism, particularly an animal, more particularly a mammal including a human, only when conjugated to a macromolecular carrier such as a protein. A “hapten-like compound” is a small molecule that resembles the structure of a given hapten sufficiently enough to bind to the corresponding anti-hapten antibody.

In most antibodies, a binding fragment binds to the same antigen or hapten as was used as an immunogen to generate the antibody. The 38C2 antibody was generated with a β-diketone hapten (Hwang et al., Biomolecules 10, 764, 2020). Cys and Arg variants of humanized 38C2 antibody at Kabat position 93 do not bind this hapten but do bind small molecules like the hapten (hapten-like compounds) including maleimide, monobromomaleimide, or dibromomaleimide, which bind to the Cys variant, and phenylglyoxal (PGO), glyoxal (GO), and methylglyoxal (MGO), which bind the Arg variant. Thus, a binding fragment of the 38C2 antibody includes fragments binding to its hapten and hapten-like compounds.

An “antigen/hapten-binding site” or “antigen/hapten-binding region” of an immunoglobulin of the present invention typically contains six complementarity determining regions (CDRs) within each variable domain, and which contribute in varying degrees to the affinity of the binding site for antigen. In each variable domain there are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences and/or structural information from antibody/antigen complexes. Also included within the scope of the invention are functional antigen or hapten binding sites comprised of fewer CDRs (i.e., where binding specificity is determined by three, four or five CDRs). Less than a complete set of 6 CDRs can be sufficient for binding to some binding targets. Thus, in some instances, the CDRs of a V_{H} or a V_{L} domain alone will be sufficient. Furthermore, certain antibodies might have non-CDR-associated binding sites for an antigen. Such binding sites are specifically included within the present definition.

The assignment of amino acids to each V_{L} and V_{H} domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al (bioinfo.org.uk/abs), and IMGT definition (imgt.org/IMGTScientificChart/Numbering/IMGTnumberingCDR_VK.html) (see Table 1). Kabat provides a widely used numbering convention (Kabat numbering) in which corresponding residues between different heavy chains or between different light chains are assigned the same number. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR H1 is a composite Chothia-Kabat CDR H1 and other CDRs contain Kabat CDR residues and no additional CDR residues based on other definitions.

TABLE 1
Conventional Definitions of CDRs Using Kabat Numbering

| | | | Composite | | | |
|---|---|---|---|---|---|---|
| | | | of Chothia | | | |
| Loop | Kabat | Chothia | & Kabat | AbM | Contact | IMGT |
| L1 | L24--L34 | L24--L34 | L24--L34 | L24--L34 | L30--L36 | L27-L32 |
| L2 | L50--L56 | L50--L56 | L50--L56 | L50--L56 | L46--L55 | L50-L52 |
| L3 | L89--L97 | L89--L97 | L89--L97 | L89--L97 | L89--L96 | L89-L97 |
| H1 | H31--H35B | H26-- | H26--H35B* | H26--H35B | H30--H35B | H26-H33 |
| | | H32 . . . H34* | | | | |
| H2 | H50--H65 | H52--H56 | H50--H65 | H50--H58 | H47--H58 | H51-H56 |
| H3 | H95--H102 | H95--H102 | H95--H102 | H95--H102 | H93--H101 | H93-H102 |

*CDR-H1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia CDR-H1 loop ends at H32. If only H35A is present, it ends at H33. If both H35A and H35B are present, it ends at H34.

The term “host cell” as used in the current application denotes any kind of cellular system which can be engineered to generate the immunoglobulins according to the current invention. In one aspect, Chinese hamster ovary (CHO) cells are used as host cells. In some embodiments, E. coli can be used as host cells.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

Percentage sequence identities between antibody sequences can be determined with antibody sequences maximally aligned by the Kabat numbering convention (or Eu index for the heavy chain constant region). After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for cancer, if, after receiving a therapeutic amount of a subject immunoconjugate according to the methods of the present invention, the subject shows observable and/or measurable reduction in or absence of one or more of the following: reduction in the number of cancer cells or absence of the cancer cells; reduction in the tumor size; inhibition (i.e., slowing to some extent and preferably stopping) of cancer cell infiltration into peripheral organs, including the spread of cancer into soft tissue and bone; inhibition (i.e., slowing to some extent and preferably stopping) of tumor metastasis; inhibition, to some extent, of tumor growth; and/or relief to some extent of one or more of the symptoms associated with the specific cancer; reduced morbidity and/or mortality, and improvement in quality of life issues.

An “antibody compound” can be a either a naked or conjugated antibody, in which the antibody can be a native antibody, binding fragment, or combination of forms as in a DVD or TVD.

### Iii. Antibody Compounds with Reactive Cys Residue(s)

The invention provides antibody compounds that contain a variant of catalytic antibody 38C2 or hapten-binding fragments thereof. The 38C2 catalytic antibody is well known in the art and has been well characterized in, e.g., U.S. Pat. No. 8,252,902. 38C2 should be understood as referring to any of the mouse antibody having a heavy chain variable region of SEQ ID NO:16 and a light chain variable region of SEQ ID NO: 17; a humanized antibody having a heavy chain variable region of SEQ ID NO:3 and a light chain variable region of SEQ ID NO:2; a humanized antibody having a heavy chain variable region of SEQ ID NO: 1 and a light chain variable region of SEQ ID NO:2; a humanized antibody having a heavy chain variable region of SEQ ID NO:4 and a light chain variable region of SEQ ID NO: 2; or any other antibody sharing six CDRs of the 38C2 antibody from SEQ ID NOs: 16 and 17.

VH of Mouse 38C2
(SEQ ID NO: 16)
Kabat CDRs in boldface
EVKLVESGGGLVQPGGTMKLSCEISGLTFRNYWMSWVRQSPEKGLEWVAE
IRLRSDNYATHYAESVKGKFTISRDDSKSRLYLQMNSLRTEDTGIYYCKT
YFYSFSYWGQGTLVTVSA
VL of Mouse 38C2
(SEQ ID NO: 17)
Kabat CDRs in boldface
DVVMTQTPLSLPVRLGDQASISCRSSQSLLHTYGSPYLNWYLQKPGQSPK
LLIYKVSNRFSGVPDRESGSGSGTDFTLRISRVEAEDLGVYFCSQGTHLP
YTFGGGTKLEIK
Mouse 38C2 Kabat CDR-H1
(SEQ ID NO: 18)
NYWMS
Mouse 38C2 Kabat CDR-H2
(SEQ ID NO: 19)
EIRLRSDNYATHYAESVKG
Mouse 38C2 Kabat CDR-H3
(SEQ ID NO: 20)
YFYSFSY
Mouse 38C2 Kabat CDR-L1
(SEQ ID NO: 21)
RSSQSLLHTYGSPYLN
Mouse 38C2 Kabat CDR-L2
(SEQ ID NO: 22)
KVSNRFS
Mouse 38C2 Kabat CDR-L3
(SEQ ID NO: 23)
SQGTHLPYT

Exemplary amino acid sequences of the heavy and light chain variable regions of a humanized 38C2 catalytic antibody are SEQ ID NOs: 3 and 2, respectively. CDR-H1, H2 and H3 by Kabat definition are assigned SEQ ID NOs: 18-20 and CDR-L1, L2 and L3 by Kabat definition are assigned SEQ ID NOs: 21-23.

The heavy chain variable region of the 38C2 antibody includes a single, uniquely reactive lysine residue (Lys99 of SEQ ID NO:3 by sequential numbering) that can react with a linker, thereby providing an attachment point for conjugation with a drug moiety. As such, immunoglobulin molecules that include a variable domain of the 38C2 antibody contain two such attachment points (one on each heavy chain) that can be used for conjugation with a drug moiety. Once a reactive lysine residue has been conjugated to a linker, the binding functionality of the 38C2 variable domain is lost, meaning that the variable domain no longer exhibits catalytic activity.

Reference to position 99 in any other heavy chain variable region sequence means corresponding position when the other sequence is maximally aligned with SEQ ID NO.3. The position in the other heavy chain variable sequence may or may not be the 99th amino acid by sequential numbering depending on whether the number of amino acids is the same in the respective sequences. Equivalently position 99 of SEQ ID NO: corresponds to position 93 by Kabat numbering in SEQ ID NO:3 and any other sequence, because Kabat numbering automatically assigns the same number to corresponding positions.

The 38C2 variant antibodies of the invention contain a cysteine substitution for this reactive lysine residue in the hydrophobic cleft, which provides an attachment point for drug conjugation that is different from the reactive lysine residue. For full length antibodies (e.g., IgG) or dimeric antibody fragments (e.g., F(ab′)₂), the substitution can be present in one or both antibody arms or antigen/hapten-binding sites. With an appropriate linker moiety, the engineered Cys residue in the 38C2 variant (38C2_Cys) is able to react with the drug moiety to form an ADC. Thus, in some embodiments, both variant domains of the antibody have the reactive Lys residue replaced with Cys. In some embodiments, the 38C2_Cys variant antibody can contain a reactive Cys residue in one of its two binding arms and a reactive Lys residue in the other binding arm. In some embodiments, the 38C2_Cys variant antibody can contain a reactive Cys residue in one of its two binding arms and a reactive Arg residue in the other binding arm. In some embodiments, the 38C2_Cys variant employed in the ADCs of the invention is a chimeric antibody. In some other embodiments, the 38C2_Cys variant used in the invention is a humanized antibody (h38C2_Cys). In various embodiments, the 38C2_Cys variant can contain a humanized light sequence, a humanized heavy chain sequence or both. The ADCs of the invention are homogeneous due to site-specific conjugation to the reactive Cys, Lys, and Arg residues.

Antibody compounds containing a variant 38C2 antibody with the reactive Lys residue replaced by Cys can be readily produced via routinely practiced methods, e.g., recombinant expression as exemplified herein. As specific exemplification, the heavy and light chain variant domain sequences of a humanized 38C2_Cys variant (h38C2_Cys) suitable for the invention are shown in SEQ ID NOs: 1 and 2, respectively. The substituted Cys residue at position 99 is underlined in the heavy chain sequence (SEQ ID NO:1). It is noted that the light chain variable domain sequence of this variant is identical to the light chain variable domain sequence of humanized 38C2 antibody (h38C2) known in the art (SEQ ID NO: 2).

V_{H} of h38C2_Cys
(SEQ ID NO: 1)
CDR-H1 (Kabat 31-35), CDR-H2 (Kabat 50-65), and
CDR-H3 (Kabat 95-102) are shown in bold. The K99C
mutation (Kabat 93) is underlined.
EVQLVESGGGLVQPGGSLRLSCAASGFTFSNYWMSWVRQSPEKGLEWVSE
IRLRSDNYATHYAESVKGRFTISRDNSKNTLYLQMNSLRAEDTGIYYCCT
YFYSFSYWGQGTLVTVSS
V_{κ} of h38C2_Cys CDR-L1 (Kabat 24-34), CDR-L2
(SEQ ID NO: 2)
(Kabat 50-56), and CDR-L3 (Kabat 89-97) are shown
in bold.
ELQMTQSPSSLSASVGDRVTITCRSSQSLLHTYGSPYLNWYLQKPGQSPK
LLIYKVSNRFSGVPSRFSGSGSGTDFTLTISSLQPEDFAVYFCSQGTHLP
YTFGGGTKVEIK

The heavy chain sequence of humanized 38C2 antibody (Lys) is provided as SEQ ID NO: 3. The heavy chain variant domain sequences of a humanized 38C2_Arg variant (h38C2_Arg) suitable for the invention is shown as SEQ ID NO:4. It is noted that the light chain variable domain sequence of humanized 38C2_Arg variant (h38C2_Arg) variant is identical to the light chain variable domain sequence of humanized 38C2 antibody (h38C2) known in the art (SEQ ID NO:2).

V_{H} of h38C2_Lys
(SEQ ID NO: 3)
CDR-H1 (Kabat 31-35), CDR-H2 (Kabat 50-65), and
CDR-H3 (Kabat 95-102) are shown in bold. K99
(Kabat 93) is underlined.
EVQLVESGGGLVQPGGSLRLSCAASGFTFSNYWMSWVRQSPEKGLEWVSE
IRLRSDNYATHYAESVKGRFTISRDNSKNTLYLQMNSLRAEDTGIYYCKT
YFYSFSYWGQGTLVTVSS
V_{H} of h38C2_Arg
(SEQ ID NO: 4)
CDR-H1 (Kabat 31-35), CDR-H2 (Kabat 50-65), and
CDR-H3 (Kabat 95-102) are shown in bold. The K99R
mutation (Kabat 93) is underlined.
EVQLVESGGGLVQPGGSLRLSCAASGFTFSNYWMSWVRQSPEKGLEWVSE
IRLRSDNYATHYAESVKGRFTISRDNSKNTLYLQMNSLRAEDTGIYYCRT
YFYSFSYWGQGTLVTVSS

In various embodiments, the antibody compounds of the invention can contain one or two or three reactive Cys residues noted above, and with a heavy chain and/or light chain sequences that are substantially identical to the exemplified sequences. For example, other than the presence of the reactive Cys residue(s), the heavy chain and light chain variable domain amino acid sequences of the antibody compounds of the invention can be of at least about 80% identical, alternatively at least about 81%, 82%, 83%, 84%, 85%, 86%, 87%, 88%, 89%, 90%, 91%, 92%, 93%, 94%, 95%, 96%, 97%, 98%, or 99% identical to SEQ ID NOs: 1 and 2, respectively.

### Iv. Antibody Conjugated Drugs with Site-Specific Cys Conjugation

The invention also provides antibody conjugated drugs (ADCs) that contain at least one drug moiety that is site-specifically conjugated to an antibody compound via an engineered cysteine residue. Preferably, the antibody compound is a variant derived from catalytic antibody 38C2 noted above. In some embodiments, the antibody compound is a homodimeric molecule that contains the Lys99Cys substitution in both antibody arms. In these embodiments, the ADCs can contain the same drug moiety that is conjugated to the engineered Cys residue in both arms of the antibody compound. In some embodiments, the antibody compound is a heterodimeric molecule that contains the Lys99Cys substitution in just one antibody arm. Heavy chain heterodimerization for such molecules can be accomplished, e.g., via knobs-into-holes mutations as exemplified herein. In some embodiments, the ADCs can contain a first drug moiety that is conjugated to the engineered Cys residue in one antibody arm and a second drug moiety that is conjugated to the reactive Lys residue in the other antibody arm. In other embodiments, the ADCs can contain a first drug moiety that is conjugated to the engineered Cys residue in one antibody arm and a second drug moiety that is conjugated to an engineered reactive Arg residue in the other antibody arm. In some embodiments, the antibody compound in the ADCs is a humanized 38C2_Cys antibody (h38C2_Cys) or hapten-binding fragment thereof alone. In some other embodiments, the antibody compound is a dual variable domain (DVD) compound (DVD-Fab or DVD-Ig as exemplified herein) or a triple variable domain (TVD) compound (TVD-Fab or TVD-Ig as exemplified herein) or a bispecific antibody that harbors 38C2_Cys. Thus, the antibody compound in some ADCs of the invention is a DVD-Ig that contains a first variable domain that binds to a target antigen (e.g., a tumor cell surface antigen or receptor) and a second variable domain (38C2_Cys) that allows site-specific attachment of a linker molecule or linker-derivatized drug moiety. In some other ADCs of the invention is a TVD-Ig that contains a first variable domain that binds to a target antigen (e.g., a tumor cell surface antigen or receptor), a second variable domain (38C2_Cys) that allows site-specific attachment of a linker molecule or linker-derivatized drug moiety, and a third variable domain (38C2_Arg, 38C2_Lys, or a second 38C2_Cys) that allows site-specific attachment of a linker molecule or linker-derivatized drug moiety. In some ADCs of the invention is a TVD-Ig, wherein the outer Fv recognizes a first target antigen (e.g., a tumor cell surface antigen or receptor), the upper inner Fv recognizes a second target antigen (e.g., a tumor cell surface antigen or receptor), and the lower inner Fv comprises h38C2_K99C that allows site-specific attachment of a linker molecule or linker-derivatized drug moiety, wherein the first and second target antigens are different from each other.

Once the variant 38C2 antibody containing reactive Cys is generated, DVD-Ig or TVD-Ig antibody compounds containing the 38C2_Cys antibody can be produced in accordance with methods that have been reported in the literature. See, e.g., Nanna et al., Nat. Commun. 8:1112, 2017; and WO2017/049139. The DVD-Ig to which drug moieties are conjugated contains a first variable domain, the 38C2 variant, for attachment of drug moieties, and a second variable domain for binding to a target of interest. Some TVD-Ig to which drug moieties are conjugated contain a first variable domain, a 38C2 variant or 38C2_Lys, for attachment of drug moieties, a second variable domain, a 38C2 variant or 38C2_Lys, for attachment of drug moieties, and a third variable domain for binding to a target of interest. When the antibody component of the ADCs has an intact antibody structure, the DVD-Ig or TVD-Ig typically contains two arms, each consisting of a light chain and a heavy chain. Each light chain and each heavy chain includes an N-terminus and a C-terminus. In some embodiments, the two arms of the DVD-Ig or TVD-Ig are identical, i.e., with the light chains being identical and the heavy chains being identical. For example, some of these embodiments are directed to homodimeric DVD compounds (e.g., homodimeric HER2 targeting DVD-Ig or TVD-Ig molecules as exemplified herein) that harbor a variant 38C2 antibody containing two h38C2_Cys arms. In some other embodiments, the two arms of the DVD-Ig or TVD-Ig can be different. For example, some of the DVD compounds can be heterodimeric in that the variant 38C2 antibody component of the DVD compounds contains one h38C2_Lys arm and one h38C2_Cys arm. In another example, some of the DVD compounds can be heterodimeric in that the variant 38C2 antibody component of the DVD compounds contains one h38C2_Arg arm and one h38C2_Cys arm.

In another example, some of the DVD compounds can be heterodimeric, wherein a first arm contains a target-binding Fv and a h38C2_Cys Fv and a second arm contains a h38C2_Lys Fv and a h38C2_Arg Fv. Some other DVD can be heterodimeric, wherein a first arm contains a target-binding Fv and a h38C2_Lys Fv and a second arm contains a h38C2_Cys Fv and a h38C2_Arg Fv. Some other DVD compounds can be heterodimeric, wherein a first arm contains a target-binding Fv and a h38C2_Arg Fv and a second arm contains a h38C2_Cys Fv and a h38C2_Lys Fv.

For example, some of these embodiments are directed to homodimeric TVD compounds that harbor a variant 38C2 antibody wherein each arm of the TVD contains two h38C2_Cys Fv and a target-binding Fv. Other embodiments are directed to homodimeric TVD compounds that harbor a variant 38C2 antibody wherein each arm of the TVD contains one h38C2_Cys Fv and one h38C2_Lys Fv and a target-binding Fv. Other embodiments are directed to homodimeric TVD compounds that harbor a variant 38C2 antibody wherein each arm of the TVD contains one h38C2_Cys Fv and one h38C2_Arg Fv and a target-binding Fv.

In some other embodiments, the two arms of the TVD-Ig can be different. For example, some of the TVD compounds be heterodimeric wherein the variant 38C2 antibody component of the TVD compound contains in a first arm two h38C2_Cys Fv and a target-binding Fv and contains in a second arm two Fvs in a combination selected from the group consisting of (1) one h38C2_Cys Fv and one h38C2_Lys Fv; (2) one h38C2_Cys Fv and one h38C2_Arg Fv; (3) two h38C2_Lys Fv; (4) two h38C2_Arg Fv; and (5) one h38C2_Lys Fv and one h38C2_Arg Fv, and a target-binding Fv. In another example, some of the TVD compounds can be heterodimeric wherein the variant 38C2 antibody component of the TVD compounds contains in a first arm one h38C2_Cys Fv and one h38C2_Lys Fv and a target-binding Fv and contains in a second arm two Fvs in a combination selected from the group consisting of (1) one h38C2_Cys Fv and one h38C2_Arg Fv; (2) two h38C2_Lys Fv; (3) two h38C2_Arg Fv; and (4) one h38C2_Lys Fv and one h38C2_Arg Fv and a target-binding Fv. In another example, some of the TVD compounds can be heterodimeric wherein one arm of the TVD compound contains in a first arm one h38C2_Cys Fv and one h38C2_Arg Fv and a target-binding Fv and contains in a second arm two Fvs in a combination selected from the group consisting of (1) one h38C2_Cys Fv and one h38C2_Lys Fv; (2) two h38C2_Lys Fv; (3) two h38C2_Arg Fv; and (4) one h38C2_Lys Fv and one h38C2_Arg Fv and a target-binding Fv.

In some DVD embodiments, heavy and light chain variable regions of the second variable domain are linked to N-termini of the heavy and light chain variable regions of the 38C2 variant domain. In some other embodiments, heavy and light chain variable regions of the 38C2 variant domain are linked to N-termini of the heavy and light chain variable regions of the second variable domain.

In some embodiments, the DVD-Ig contains 38C2_Cys as the first variable domain for conjugating the drug moieties and a second variable domain that binds to a target of interest (e.g., a target antigen or receptor). In some of these ADCs, the reactive Cys is present in both arms of the 38C2-Cys variant and identical drug moieties are conjugated to the two arms of the antibody compound. In some other ADCs, the reactive lysine residue in only one arm of the 38C2 variant antibody is replaced with a cysteine residue. These ADCs contain both a reactive Cys and a reactive Lys in the two arms, to which 2 different drug moieties are respectively conjugated via appropriate linkers, as exemplified herein. In some other ADCs, the reactive lysine residue in one arm of the 38C2 variant antibody is replaced with a cysteine residue and the other is replaced with an arginine residue. These ADCs contain both a reactive Cys and a reactive Arg in the two arms, to which 2 different drug moieties are respectively conjugated via appropriate linkers, as exemplified herein.

In some other ADCs, one arm of the DVD-Ig comprises a first variable domain comprising a reactive Cys and a second variable domain comprising a reactive Arg, and the other arm of the DVD-Ig comprises a first variable domain comprising a reactive Lys and a second variable domain comprising a target-binding domain. In some other ADCs, one arm of the DVD-Ig comprises a first variable domain comprising the reactive Cys and a second variable domain comprising the reactive Lys, and the other arm of the DVD-Ig comprises a first variable domain comprising a reactive Arg and a second variable domain comprising a target-binding domain. In some other ADCs, one arm of the DVD-Ig comprises a first variable domain comprising the reactive Arg and a second variable domain comprising the reactive Lys, and the other arm of the DVD-Ig comprises a first variable domain comprising a reactive Cys and a second variable domain comprising a target-binding domain. These ADCs contain a reactive Cys, a reactive Arg, and a reactive Lys in the two arms, to which 3 different drug moieties are respectively conjugated via appropriate linkers, as exemplified herein.

In some TVD embodiments, heavy and light chain variable regions of a target-binding variable domain are linked to N-termini of the heavy and light chain variable regions of a 38C2 variant domain. In some other embodiments, heavy and light chain variable regions of a 38C2 variant domain are linked to N-termini of the heavy and light chain variable regions of a target-binding variable domain.

In some embodiments, the TVD-Ig contains 38C2_Cys as the first and/or second variable domain for conjugating the drug moieties and a third variable domain that binds to a target of interest (e.g., a target antigen or receptor). In some of these ADCs, the reactive Cys is present in the first and second variable domains in both arms of the TVD-Ig and identical drug moieties are conjugated to the two arms of the antibody compound. In some other ADCs, each arm of the TVD-Ig comprises a first variable domain comprising the reactive Cys and a second variable domain comprising the reactive Lys. These ADCs contain both a reactive Cys and a reactive Lys in the two arms, to which 2 different drug moieties are respectively conjugated via appropriate linkers, as exemplified herein. In some other ADCs, each arm of the TVD-Ig comprises a first variable domain comprising the reactive Cys and a second variable domain comprising the reactive Arg. These ADCs contain both a reactive Cys and a reactive Arg in the two arms, to which 2 different drug moieties are respectively conjugated via appropriate linkers, as exemplified herein. In some other ADCs, one arm of the TVD-Ig comprises a first variable domain comprising the reactive Cys and a second variable domain comprising the reactive Arg, and the other arm of the TVD-Ig comprises a first variable domain comprising a reactive Cys and a second variable domain comprising a reactive Lys. These ADCs contain two reactive Cys, a reactive Arg, and a reactive Lys in the two arms, to which 3 different drug moieties are respectively conjugated via appropriate linkers, as exemplified herein.

Immunoglobulins of variant types or subtypes can be used in the constructions of the DVD-Ig or TVD-Ig antibody compounds of the invention. For example, the light chain can be a kappa light chain or a lambda light chain. Depending on the Fc domain, the heavy chain can be that from an IgG (such as an IgG1, IgG2, IgG3 or IgG4), IgA (such as an IgA1 or IgA2), IgM, IgE or IgD antibody. For example, in some aspects, an immunoglobulin belongs to the IgG class, and the heavy chain comprises a γ heavy chain. In some embodiments, an immunoglobulin belongs to the IgG1 class, and the heavy chain comprises a γ1 heavy chain. In some embodiments, an immunoglobulin belongs to the IgG2 class, and the heavy chain comprises a γ2 heavy chain. In some embodiments, an immunoglobulin belongs to the IgG3 class, and the heavy chain comprises a γ3 heavy chain. In some embodiments, an immunoglobulin belongs to the IgG4 class, and the heavy chain comprises a γ4 heavy chain. In some embodiments, an immunoglobulin belongs to the IgA class, and a heavy chain comprises an a heavy chain. In some embodiments, an immunoglobulin belongs to the IgA1 class, and a heavy chain comprises a α1 heavy chain. In some embodiments, an immunoglobulin belongs to the IgA2 class, and a heavy chain comprises a α2 heavy chain. In some embodiments, an immunoglobulin belongs to the IgD class, and a heavy chain comprises a δ heavy chain. In some embodiments, an immunoglobulin belongs to the IgE class, and a heavy chain comprises an ε heavy chain. In some embodiments, an immunoglobulin belongs to the IgM class, and a heavy chain comprises a μ heavy chain.

In various embodiments, the first and second variable domains of the DVD-Ig compounds or the first, second, and third variable domains of the TVD-Ig antibody compounds are linked along their light chain or heavy chain by a peptide linker sequence. A peptide linker sequence can be a single amino acid or a polypeptide sequence. A number of linkers that can be employed in the present invention are described in the art, e.g., WO2017/049139 and U.S. Pat. No. 7,612,181. Some specific examples of suitable linkers include ASTKGP (SEQ ID NO:5) and TVAAPSVFIFPP (SEQ ID NO:6).

The second variable domain of the DVD-Ig or TVD-Ig in the ADCs of the invention can be any antibody or antigen-binding fragment that specifically recognizes a target polypeptide or target antigen of interest. For example, it can be an antibody, antibody domain or antigen-binding fragment that recognizes an antigen on a tumor cell. Immunoglobulins can exert antitumor effects by inducing apoptosis, redirected cytotoxicity, interfering with ligand-receptor interactions, or preventing the expression of proteins that are critical to a neoplastic phenotype. In addition, immunoglobulins can target components of the tumor microenvironment, perturbing vital structures such as the formation of tumor-associated vasculature. Immunoglobulins can also target receptors whose ligands are growth factors, such as the epidermal growth factor receptor, thus inhibiting binding of natural ligands that stimulate cell to targeted tumor cells. Alternatively, immunoglobulins can induce ADCC, ADCP or CDC.

One of skill in the art will realize that tumor-associated antigens are known for virtually any type of cancer. Specific tumor-associated binding targets that can be targeted by the second variable domain of a subject DVD or TVD immunoglobulin molecule include HER2 (ERBB2) as exemplified herein. Other examples include, but are not limited to, FOLR1, FOLR2, CD138, CD19, CD79A, CD79B, ROR1, ROR2, FCMR (TOSO), CS1, GPA33, MSLN, CD52, CD20, CD3, CD4, CD5, CD8, CD20, CD21, CD22, CD23, CD30, CD33, CD38, CD44, CD56, CD70, CD123, BCMA, Siglec-1, Siglec-4, Siglec-5, Siglec-6, Siglec-7, Siglec-8, Siglec-9, Siglec-15, PSMA, BMP6, IL12A, IL1A, IL1B, IL2, IL24, INHA, TNF, TNFSF10, BMP6, EGF, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, FGFR4, GD2, tissue factor, TROP2, Nectin-4, fibronectin, extra-domain A fibronectin, extra-domain B fibronectin, DLK1, PDL1, PDL2, B7H3, B7H4, GRP, IGF1, IGF2, IL12A, IL1A, IL1B, IL2, INHA, TGFA, TGFB1, TGFB2, TGFB3, VEGF, CDK2, EGF, FGF2, FGF4, FGF7, IGF1, IGF1R, IL2, VEGF, BCL2, CD164, CDKN1A, CDKN1B, CDKN1C, CDKN2A, CDKN2B, CDKN2C, CDKN3, GNRH1, IGFBP6, IL1A, IL1B, ODZ1, PAWR, PLG, TGFB11, AR, BRCA1, CDK3, CDK4, CDK5, CDK6, CDK7, CDK9, E2F1, EGFR (ERBB1), EGFRvIII, HER3 (ERBB3), HER4 (ERBB4), ENO1, ESR1, ESR2, IGFBP3, IGFBP6, IL2, INSL4, MYC, NOX5, NR6A1, PAP, PCNA, PRKCQ, PRKD1, PRL, TP53, FGF22, FGF23, FGF9, IGFBP3, IL2, INHA, KLK6, TP53, CHGB, GNRH1, IGF1, IGF2, INHA, INSL3, INSL4, PRL, KLK6, SHBG, NR1D1, NR1H3, NR113, NR2F6, NR4A3, ESR1, ESR2, NROB1, NROB2, NR1D2, NR1H2, NR1H4, NR112, NR2C1, NR2C2, NR2E1, NR2E3, NR2F1, NR2F2, NR3C1, NR3C2, NR4A1, NR4A2, NR5A1, NR5A2, NR6A1, PGR, RARB, FGF1, FGF2, FGF6, KLK3, KRT1, APOC1, BRCA1, CHGA, CHGB, CLU, COL1A1, COL6A1, IGFBP3, IGFBP6, IL12A, IL1A, IL1B, IL2, IL24, INHA, INSL3, INSL4, KLK10, KLK12, KLK13, KLK14, KLK15, KLK3, KLK4, KLK5, KLK6, KLK9, MMP2, MMP9, MSMB, NTN4, ODZ1, PAP, PLAU, PRL, PSAP, SERPINA3, SHBG, TGFA, TIMP3, CD44, CDH1, CDH10, CDH19, CDH20, CDH7, CDH9, CDH1, CDH10, CDH13, CDH18, CDH19, CDH20, CDH7, CDH8, CDH9, ROBO2, CD44, ILK, ITGA1, APC, CD164, COL6A1, MTSS1, PAP, TGFB1I1, AGR2, AIG1, AKAP1, AKAP2, CANT1, CAV1, CDH12, CLDN3, CLN3, CYB5, CYC1, DAB21P, DES, DNCL1, ELAC2, ENO2, ENO3, FASN, FLJ12584, FLJ25530, GAGEB1, GAGEC1, GGT1, GSTP1, HIP1, HUMCYT2A, IL29, K6HF, KAI1, KRT2A, MIB1, PART1, PATE, PCA3, PIAS2, PIK3CG, PPID, PR1, PSCA, SLC2A2, SLC33A1, SLC43A1, STEAP, STEAP2, TPM1, TPM2, TRPC6, ANGPT1, ANGPT2, ANPEP, ECGF1, EREG, FGF1, FGF2, FGF1, FLT1, JAG1, KDR, LAMA5, NRP1, NRP2, PGF, PLXDC1, STAB1, VEGF, VEGFC, ANGPTL3, BAI1, COL4A3, IL8, LAMA5, NRP1, NRP2, STAB1, ANGPTL4, PECAM1, PF4, PROK2, SERPINF1, TNFAIP2, CCL11, CCL2, CXCL1, CXCL10, CXCL3, CXCL5, CXCL6, CXCL9, IFNA1, IFNB1, IFNG, IL1B, IL6, MDK, EDG1, EFNA1, EFNA3, EFNB2, EGF, EPHB4, FGFR3, HGF, IGF1, ITGB3, PDGFA, TEK, TGFA, TGFB1, TGFB2, TGFBR1, CCL2, CDH5, COL18A1, EDG1, ENG, ITGAV, ITGB3, ITGAV/ITGB3 (avb3 integrin), ITGB5, ITGAV/ITGB5 (avb5 integrin), ITGA4, ITGB1, ITGA4/ITGB1 (a4b1 integrin), THBS1, THBS2, BAD, BAG1, BCL2, CCNA1, CCNA2, CCND1, CCNE1, CCNE2, CDH1 (E-cadherin), CDKN1B (p27Kip1), CDKN2A (p16INK4a), COL6A1, CTNNB1 (b-catenin), CTSB (cathepsin B), ESR1, ESR2, F3 (TF), FOSL1 (FRA-1), GATA3, GSN (Gelsolin), IGFBP2, IL2RA, IL6, IL6R, IL6ST (glycoprotein 130), ITGA6 (a6 integrin), JUN, KLK5, KRT19, MAP2K7 (c-Jun), MKI67 (Ki-67), NGFB (NGF), NGFR, NME1 (NM23A), PGR, PLAU (uPA), PTEN, SERPINB5 (maspin), SERPINE1 (PAI-1), TGFA, THBS1 (thrombospondin-1), TIE (Tie-1), TNFRSF6 (Fas), TNFSF6 (FasL), TOP2A (topoisomerase Iia), TP53, AZGP1 (zinc-a-glycoprotein), BPAG1 (plectin), CDKN1A (p21Wap1/Cip1), CLDN7 (claudin-7), CLU (clusterin), FGF1, FLRT1 (fibronectin), GABRP (GABAa), GNAS1, ID2, ITGA6 (a6 integrin), ITGB4 (b4 integrin), KLF5 (GC Box BP), KRT19 (Keratin 19), KRTHB6 (hair-specific type II keratin), MACMARCKS, MT3 (metallothionectin-III), MUC1 (mucin), PTGS2 (COX-2), RAC2 (p21Rac2), S100A2, SCGB1D2 (lipophilin B), SCGB2A1 (mammaglobin 2), SCGB2A2 (mammaglobin 1), SPRR1B (Spr1), THBS1, THBS2, THBS4, TNFAIP2 (B94), and SLAMF7.

The amino acid sequences of the second variable domain of the DVD-Ig or in the first and second variable domains of the TVD-Ig in the ADCs of the invention can include chimeric, humanized, or human amino acid sequences. Any suitable combination of such sequences can be incorporated into the second variable domain of the DVD-Ig antibody compounds of the invention or into the first and second variable domains of the TVD-Ig antibody compounds of the invention.

Antigen-binding variable region sequences can be selected from various monoclonal antibodies capable of binding specific targets and well known in the art. These include, but are not limited to anti-TNF antibody (U.S. Pat. No. 6,258,562), anti-IL-12 and or anti-IL-12p40 antibody (U.S. Pat. No. 6,914,128); anti-IL-18 antibody (US 2005/0147610 A1), anti-C5, anti-CBL, anti-CD147, anti-gp120, anti-VLA4, anti-CD11a, anti-CD18, anti-VEGF, anti-CD40L, anti-Id, anti-ICAM-1, anti-CXCL13, anti-CD2, anti-EGFR, anti-TGF-beta 2, anti-E-selectin, anti-Fact VII, anti-Her2/neu, anti-F gp, anti-CD11/18, anti-CD14, anti-ICAM-3, anti-CD80, anti-CD4, anti-CD3, anti-CD23, anti-beta2 integrin, anti-alpha4beta7 integrin, anti-alpha4beta1 integrin, anti-alphavbeta3 integrin, anti-alphavbeta5 integrin, anti-CD52, anti-HLA DR, anti-CD22, anti-Siglec-1, anti-Siglec-4, anti-Siglec-5, anti-Siglec-6, anti-Siglec-7, anti-Siglec-8, anti-Siglec-9, anti-Siglec-15, anti-PSMA, anti-BCMA, anti-ROR1, anti-ROR2, anti-DLL3, anti-FOLR1, anti-FOLR2, anti-CD5, anti-SLAMF7, anti-FCMR, anti-CD20, anti-MIF, anti-CD64 (FcuR1), anti-TCR alpha beta, anti-CD2, anti-Hep B, anti-CA 125, anti-EpCAM, anti-gp120, anti-CMV, anti-gpIIbIIIa, anti-IgE, anti-CD25, anti-CD33, anti-HLA, anti-VNRintegrin, anti-IL-1alpha, anti-IL-1beta, anti-IL-1 receptor, anti-IL-2 receptor, anti-IL-4, anti-IL4 receptor, anti-IL5, anti-IL-5 receptor, anti-IL-6, anti-IL-8, anti-IL-9, anti-IL-13, anti-IL-13 receptor, anti-IL-17, anti-IL-23, anti-FCMR (TOSO), anti-CD123, anti-FGFR4, anti-GD2, anti-tissue factor, anti-TROP2, anti-Nectin-4, anti-fibronectin, anti-extra-domain A fibronectin, anti-extra-domain B fibronectin, anti-DLK1, anti-PDL1, anti-PDL2, anti-B7H3, anti-B7H4, anti-EGFRvIII, anti-ITGAV/ITGB3 (avb3 integrin), anti-ITGB5, anti-ITGAV/ITGB5 (avb5 integrin), anti-ITGA4, anti-ITGB1, anti-ITGA4/ITGB1 (a4b1 integrin), and anti-SLAMF7. See, e.g., Beck et al., Nat Rev Drug Discov. 2017, 16:315-337; Carter and Lazar, Nat Rev Drug Discov. 2018, 17:197-223; Kaplon and Reichert, MAbs. 2018, 10:183-203; Reichert, MAbs. 2017, 9:167-181; Reichert, MAbs. 2016, 8:197-204; and Presta L G., J. Allergy Clin. Immunol. 2005, 116:731-6.

Antigen-binding variable region sequences can also be selected from various therapeutic antibodies approved for use, in clinical trials, or in development for clinical use. Such therapeutic antibodies include, but are not limited to, RITUXAN®, IDEC/Genentech/Roche) (see for example U.S. Pat. No. 5,736,137), a chimeric anti-CD20 antibody approved to treat non-Hodgkin's lymphoma; HUMAX-CD20®, an anti-CD20 developed by Genmab, an anti-CD20 antibody described in U.S. Pat. No. 5,500,362, AME-133 (Applied Molecular Evolution), hA20 (Immunomedics, Inc.), HumaLYM (Intracel), and PRO70769 (PCT/US2003/040426, entitled “Immunoglobulin Variants and Uses Thereof”), trastuzumab (HERCEPTIN®, Genentech) (see for example U.S. Pat. No. 5,677,171), a humanized anti-HER2 antibody approved to treat breast cancer; pertuzumab (rhuMab-2C4, OMNITARG®), developed by Genentech; an anti-HER2 antibody described in U.S. Pat. No. 4,753,894; cetuximab (ERBITUX®, Imclone) (U.S. Pat. No. 4,943,533; PCT WO 96/40210), a chimeric anti-EGFR antibody; ABX-EGF (U.S. Pat. No. 6,235,883), developed by Abgenix-Immunex-Amgen; HUMAX-EGFR™ (U.S. Ser. No. 10/172,317), developed by Genmab; 425, EMD55900, EMD62000, and EMD72000 (Merck KGaA) (U.S. Pat. No. 5,558,864; Murthy et al. 1987, Arch Biochem Biophys. 252(2): 549-60; Rodeck et al., 1987, J Cell Biochem. 35(4): 315-20; Kettleborough et al., 1991, Protein Eng. 4(7): 773-83); ICR62 (Institute of Cancer Research) (PCT WO 95/20045; Modjtahedi et al., 1993, J. Cell Biophys. 1993, 22(1-3): 129-46; Modjtahedi et al., 1993, Br J Cancer. 1993, 67(2): 247-53; Modjtahedi et al, 1996, Br J Cancer, 73(2): 228-35; Modjtahedi et al, 2003, Int J Cancer, 105(2): 273-80); TheraCIM hR3 (YM Biosciences, Canada and Centro de Immunologia Molecular, Cuba (U.S. Pat. Nos. 5,891,996; 6,506,883; Mateo et al, 1997, Immunotechnology, 3(1): 71-81); mAb-806 (Ludwig Institute for Cancer Research, Memorial Sloan-Kettering) (Jungbluth et al. 2003, Proc Natl Acad Sci USA. 100(2): 639-44); KSB-102 (KS Biomedix); MR1-1 (IVAX, National Cancer Institute) (PCT WO 0162931A2); and SC100 (Scancell) (PCT WO 01/88138); alemtuzumab (CAMPATH®, Millennium), a humanized monoclonal antibody previously approved for treatment of B-cell chronic lymphocytic leukemia; muromonab-CD3 (Orthoclone OKT3®), an anti-CD3 antibody developed by Ortho Biotech/Johnson & Johnson, ibritumomab tiuxetan (ZEVALIN®), an anti-CD20 antibody developed by IDEC/Schering AG, gemtuzumab ozogamicin (MYLOTARG®), an anti-CD33 (p67 protein) antibody developed by Celltech/Wyeth, alefacept (AMEVIVE®), an anti-LFA-3 Fc fusion developed by Biogen), abciximab (REOPRO®), developed by Centocor/Lilly, basiliximab (SIMULECT®), developed by Novartis, palivizumab (SYNAGIS®), developed by Medimmune, infliximab (REMICADE®), an anti-TNFalpha antibody developed by Centocor, adalimumab (HUMIRA®), an anti-TNFalpha antibody developed by Abbott, HUMICADER, an anti-TNFalpha antibody developed by Celltech, etanercept (ENBREL®), an anti-TNFalpha Fc fusion developed by Immunex/Amgen, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549, 90Y-muHMFG1), an anti-MUC1 in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, ANTEGREN® (natalizumab), an anti-alpha-4-beta-1 (VLA4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-β2 antibody being developed by Cambridge Antibody Technology, J695, an anti-IL-12 antibody being developed by Cambridge Antibody Technology and Abbott, CAT-192, an anti-TGFß1 antibody being developed by Cambridge Antibody Technology and Genzyme, CAT-213, an anti-Eotaxin1 antibody being developed by Cambridge Antibody Technology, LYMPHOSTAT-B® an anti-Blys antibody being developed by Cambridge Antibody Technology and Human Genome Sciences Inc., TRAIL-R1mAb, an anti-TRAIL-RI antibody being developed by Cambridge Antibody Technology and Human Genome Sciences, Inc., AVASTIN® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, XOLAIR® (Omalizumab), an anti-IgE antibody being developed by Genentech, RAPTIVA® (Efalizumab), an anti-CD11a antibody being developed by Genentech and Xoma, MLN-02 Antibody (formerly LDP-02), being developed by Genentech and Millennium Pharmaceuticals, HUMAX CD4®, an anti-CD4 antibody being developed by Genmab, HUMAX™-IL15, an anti-IL15 antibody being developed by Genmab and Amgen, HUMAX™-Inflam, being developed by Genmab and Medarex, HUMAX™-Cancer, an anti-Heparanase I antibody being developed by Genmab and Medarex and Oxford GlycoSciences, HUMAX™-Lymphoma, being developed by Genmab and Amgen, HUMAX™-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-CIDE® (labetuzumab), an anti-carcinoembryonic antigen (CEA) antibody being developed by Immunomedics, LYMPHOCIDE® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, OSIDEM® (IDM-1), and anti-Her2 antibody being developed by Medarex and Immuno-Designed Molecules, HUMAX®-CD4, an anti-CD4 antibody being developed by Medarex and Genmab, HuMax-IL15, an anti-IL15 antibody being developed by Medarex and Genmab, CNTO 148, an anti-TNFa antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, NUVION® (visilizumab), an anti-CD3 antibody being developed by Protein Design Labs, HUZAF®, an anti-gamma interferon antibody being developed by Protein Design Labs, Anti-a 5β1 Integrin, being developed by Protein Design Labs, anti-IL-12, being developed by Protein Design Labs, ING-1, an anti-Ep-CAM antibody being developed by Xoma, XOLAIR® (Omalizumab) a humanized anti-IgE antibody developed by Genentech and Novartis, and MLN01, an anti-Beta2 integrin antibody being developed by Xoma. In some embodiments, the antigen-binding variable region sequences can be derived from any of the antibody drugs that have been approved in various therapies as shown in Table 2, which shows 49 FDA-approved antibody therapeutics. The differently formatted rows indicate mechanism of action based on natural or enhanced natural properties of mAbs (20 bolded), on engaging cytotoxic T cells (17 italicized), and on delivering cytotoxic payloads (12 underlined).

TABLE 2
FDA-approved and marketed antibody-based cancer therapy

| NAME | FORMAT | PAYLOAD | TARGET | CANCER | APPROVAL |
|---|---|---|---|---|---|
| rituximab | chimeric | none | CD20 | B-NHL, | 1997 |
| (Rituxan ®) | mouse/human | | | CLL | |
| | IgG1κ | | | | |
| trastuzumab | humanized | none | HER2 | breast, | 1998 |
| (Herceptin ®) | IgG1κ | | | stomach | |
| cetuximab | chimeric | none | EGFR | colorectal, | 2004 |
| (Erbitux ®) | mouse/human | | | h & n | |
| | IgG1κ | | | | |
| bevacizumab | humanized | none | VEGF | colorectal, | 2004 |
| (Avastin ®) | IgG1κ | | | lung, brain, | |
| | | | | kidney, | |
| | | | | cervical, | |
| | | | | ovarian, | |
| | | | | fallopian, | |
| | | | | peritoneal, | |
| | | | | liver | |
| panitumumab | human | none | EGFR | colorectal | 2006 |
| (Vectibix ®) | IgG2κ | | | | |
| ofatumumab | human | none | CD20 | CLL | 2009 |
| (Arzerra ®) | IgG1κ | | | | |
| ipilimumab | human | none | CTLA4 | melanoma, | 2011 |
| (Yervoy ®) | IgG1κ | | | kidney, MSI- | |
| | | | | H/dMMR | |
| | | | | colorectal, | |
| | | | | liver, lung, | |
| | | | | mesothelioma | |
| brentuximab | chimeric | auristatin | CD30 | HL, T-NHL | 2011 |
| vedotin | mouse/human | | | | |
| (Adcetris ®) | IgG1κ | | | | |
| pertuzumab | humanized | none | HER2 | breast | 2012 |
| (Perjeta ®) | IgG1κ | | | | |
| ado- | humanized | maytansine | HER2 | breast | 2013 |
| trastuzumab | IgG1κ | | | | |
| emtansine | | | | | |
| (Kadcyla ®) | | | | | |
| obinutuzumab | humanized | none | CD20 | CLL, | 2013 |
| (Gazyva ®) | IgG1κ | | | B-NHL | |
| | (glycoengineered | | | | |
| | Fc) | | | | |
| ramucirumab | human | none | VEGFR2 | stomach, | 2014 |
| (Cyramza ®) | IgG1κ | | | colorectal, | |
| | | | | liver, lung | |
| pembrolizumab | humanized | none | PDI | melanoma, | 2014 |
| (Keytruda ®) | IgG4κ | | | lung, h & n, | |
| | | | | HL, bladder, | |
| | | | | MSI- | |
| | | | | H/dMMR | |
| | | | | cancers, | |
| | | | | stomach, | |
| | | | | cervical, B- | |
| | | | | NHL, liver, | |
| | | | | kidney, | |
| | | | | esophageal, | |
| | | | | endometrial, | |
| | | | | TMB-H | |
| | | | | cancers, skin, | |
| | | | | breast | |
| | | | | (TNBC) | |
| blinatumomab | mouse (scFv)₂ | none | CD19 × | B-ALL | 2014 |
| (Blincyto ®) | (“BiTE” | | CD3 | | |
| | format) | | | | |
| nivolumab | human | none | PD1 | melanoma, | 2014 |
| (Opdivo ®) | IgG4κ | | | lung, kidney, | |
| | | | | HL, h & n, | |
| | | | | bladder, | |
| | | | | MSI- | |
| | | | | H/dMMR | |
| | | | | colorectal, | |
| | | | | liver, | |
| | | | | esophageal, | |
| | | | | mesothelioma, | |
| | | | | stomach | |
| dinutuximab | chimeric | none | GD2 | neuroblastoma | 2015 |
| (Unituxin ®) | mouse/human | | | | |
| | IgG1κ | | | | |
| daratumumab | human | none | CD38 | multiple | 2015 |
| (Darzalex ®) | IgG1κ | | | myeloma | |
| necitumumab | human | none | EGFR | lung | 2015 |
| (Portrazza ®) | IgG1κ | | | | |
| elotuzumab | humanized | none | SLAMF7 | multiple | 2015 |
| (Empliciti ®) | IgG1κ | | | myeloma | |
| atezolizumab | humanized | none | PDL1 | bladder, | 2016 |
| (Tecentriq ®) | IgG1κ | | | lung, breast | |
| | (aglycosylated | | | (TNBC), | |
| | Fc) | | | liver, | |
| | | | | melanoma | |
| olaratumab | human | none | PDGFRA | sarcoma | 2016 |
| (Lartruvo ®) | IgG1κ | | | | |
| avelumab | human | none | PDL1 | Merkel cell | 2017 |
| (Bavencio ®) | IgG1λ | | | carcinoma, | |
| | | | | bladder, | |
| | | | | kidney | |
| durvalumab | human | none | PDL1 | lung | 2017 |
| (Imfinzi ®) | IgG1κ | | | | |
| | (engineered | | | | |
| | Fc) | | | | |
| inotuzumab | humanized | calicheamicin | CD22 | B-ALL | 2017 |
| ozogamicin | IgG4κ | | | | |
| (Besponsa ®) | | | | | |
| tisagenlecleucel | mouse scFv- | T cell | CD19 | B-ALL, | 2017 |
| (Kymriah ®) | based CAR-T | | | B-NHL | |
| | | | | (DLBCL, FL) | |
| gemtuzumab | humanized | calicheamicin | CD33 | AML | 2017 |
| ozogamicin | IgG4κ | | | | |
| (Mylotarg ®) | | | | | |
| axicabtagene | mouse scFv- | T cell | CD19 | B-NHL | 2017 |
| ciloleucel | based CAR-T | | | (DLBCL, FL) | |
| (Yescarta ®) | | | | | |
| mogamulizumab- | humanized | none | CCR4 | T-NHL | 2018 |
| kpkc | IgG1κ | | | | |
| (Poteligeo ®) | (afucosylated | | | | |
| | Fc) | | | | |
| moxetumomab | mouse dsFv | bacterial | CD22 | B-NHL | 2018 |
| pasudotox-tdfk | | toxin | | (hairy cell | |
| (Lumoxiti ®) | | | | leukemia) | |
| cemiplimab- | human | none | PD1 | cutaneous | 2018 |
| rwlc | IgG4κ | | | squamous | |
| (Libtayo ®) | (S228P | | | cell carcinoma, | |
| | hinge) | | | basal cell | |
| | | | | carcinoma, | |
| | | | | lung | |
| polatuzumab | humanized | auristatin | CD79B | B-NHL | 2019 |
| vedotin-piiq | IgG1κ | | | (DLBCL) | |
| (Polivy ®) | | | | | |
| enfortumab | human | auristatin | NECTIN4 | bladder | 2019 |
| vedotin-ejfv | IgG1κ | | | | |
| (Padcev ®) | | | | | |
| fam- | humanized | camptothecin | HER2 | breast, | 2019 |
| trastuzumab | IgG1κ | | | stomach | |
| deruxtecan-nxki | | | | | |
| (Enhertu ®) | | | | | |
| isatuximab-irfc | chimeric | none | CD38 | multiple | 2020 |
| (Sarclisa ®) | IgG1κ | | | myeloma | |
| sacituzumab | humanized | camptothecin | TROP2 | breast | 2020 |
| govitecan-hziy | IgG1κ | | | (TNBC), | |
| (Trodelvy ®) | | | | bladder | |
| brexucabtagene | mouse scFv- | T cell | CD19 | B-NHL | 2020 |
| autoleucel | based CAR-T | | | (mantle cell | |
| (Tecartus ®) | | | | lymphoma), | |
| | | | | B-ALL | |
| tafasitamab- | humanized | none | CD19 | B-NHL | 2020 |
| cxix | IgG1κ | | | (DLBCL) | |
| (Monjuvi ®) | (engineered | | | | |
| | Fc) | | | | |
| belantamab | humanized | auristatin | BCMA | multiple | 2020 |
| mafodotin-blmf | IgG1κ | | | myeloma | |
| (Blenrep ®) | (afucosylated | | | | |
| | Fc) | | | | |
| naxitamab- | humanized | none | GD2 | neuroblastoma | 2020 |
| gqgk | IgG1κ | | | | |
| (Danyelza ®) | | | | | |
| margetuximab- | chimeric | none | HER2 | breast | 2020 |
| cmkb | IgG1κ | | | | |
| (Margenza ®) | (engineered | | | | |
| | Fc) | | | | |
| lisocabtagene | mouse scFv- | T cell | CD19 | B-NHL | 2021 |
| maraleucel | based CAR-T | | | (DLBCL, FL) | |
| (Breyanzi ®) | | | | | |
| idecabtagene | mouse scFv- | T cell | BCMA | multiple | 2021 |
| vicleucel | based CAR-T | | | myeloma | |
| (Abecma ®) | | | | | |
| dostarlimab- | humanized | none | PD1 | dMMR | 2021 |
| gxly | IgG4κ | | | endometrial, | |
| (Jemperli ®) | (S228P | | | dMMR | |
| | hinge) | | | cancers | |
| loncastuximab | chimeric | PBD | CD19 | B-NHL | 2021 |
| tesirine-lpyl | IgG1κ | dimer | | (DLBCL) | |
| (Zynlonta ®) | | | | | |
| amivantamab- | human | none | EGFR(mut) × | lung | 2021 |
| vmjw | IgG1κ | | MET | | |
| (Rybrevant ®) | (“DuoBody” | | | | |
| | format) | | | | |
| tisotumab | human | auristatin | Tissue | cervical | 2021 |
| vedotin-tftv | IgG1κ | | Factor | | |
| (Tivdak ®) | | | | | |
| tebentafusp- | human TCR × | none | MHC- | melanoma | 2022 |
| tebn | humanized | | I:gp100 × | | |
| (Kimmtrak ®) | scFv | | CD3 | | |
| | (“ImmTAC” | | | | |
| | format) | | | | |
| ciltacabtagene | llama (VHH)₂- | T cell | BCMA | multiple | 2022 |
| autoleucel | based CAR-T | | | myeloma | |
| (Carvykti ®) | | | | | |
| relatlimab- | human | none | LAG3 | melanoma | 2022 |
| rmbw | IgG4κ | | | | |
| (Opdualag ®) | | | | | |

The DVD-Ig or TVD-Ig antibody compound in the ADCs can encompass chimeric, humanized and human immunoglobulin sequences, and in some aspects, can contain any mixture thereof. In some embodiments, it can be modified with respect to effector function, e.g., so as to enhance ADCC, ADCP or CDC of the immunoglobulin. This can be achieved by introducing one or more amino acid substitutions in an Fc region of an immunoglobulin. Alternatively or additionally, cysteine residue(s) can be introduced in the Fc region, thereby allowing inter-chain disulfide bond formation in this region. An immunoglobulin thus generated can have improved internalization capability and/or increased ADCC, ADCP or CDC. See Caron et al., J. Exp Med. 176:1191-1195 (1992) and Shopes, B. J. Immunol. 148:2918-2922 (1992). To increase a serum half-life of an immunoglobulin, a salvage receptor binding epitope can be incorporated into an immunoglobulin (especially an immunoglobulin fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

As exemplification, the invention provides DVD-Ig and DVD-Fab containing 38C2_Cys for specifically targeting tumor antigen HER2. These DVD compounds contain a variable domain that binds to HER2 and a humanized 38C2_Cys variable domain as the second variable domain. The variable domains can be connected on each light and heavy chain with a peptide linker sequence, e.g., ASTKGP (SEQ ID NO:5). To facilitate recombinant protein production, a signal peptide sequence, e.g., MDWTWRILFLVAAATGAHS (SEQ ID NO:7), can be placed at the N-terminus of the heavy and light chain sequences. The light chain amino acid sequence of the DVD-Ig and DVD-Fab molecules (trastuzumab V_{κ}/ASTKGP/h38C2_Cys Vκ/C_{κ}), trastuzumab V_{κ}/ASTKGP/h38C2_Arg Vκ/C_{κ}, trastuzumab V_{κ}/ASTKGP/h38C2 Vκ/C_{κ}) exemplified herein, minus the signal peptide, is shown in SEQ ID NO: 10 (ASTKGP is SEQ ID NO:5). The heavy chain amino acid sequence of the DVD-Fab molecule (trastuzumab V_{H}/ASTKGP/h38C2_Cys V_{H}/C_{γ1}1), minus the signal peptide, is shown in SEQ ID NO:8 (ASTKGP is SEQ ID NO:5). The heavy chain amino acid sequence of the exemplified DVD-Ig molecule (trastuzumab V_{H}/ASTKGP/h38C2_Cys V_{H}/C_{γ1}1-hinge_{γ1}-C_{γ1}2-C_{γ1}3), minus the signal peptide, is shown in SEQ ID NO:9 (ASTKGP is SEQ ID NO:5). The linker sequence separating the two variable domains is underlined in these sequences. Constant region sequences are italicized in the sequences.

Heavy chain of HER2 targeting DVD-Fab
(SEQ ID NO: 8)
(trastuzumab V_{II}/ASTKGP/h38C2_Cys V_{H}/C_{γ1}1), minus
the signal peptide The K99C mutation (Kabat 93)
is underlined and bolded. (ASTKGP is SEQ ID NO: 5)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPEVQLVESGGGLVQPGGSLRLSCAA
SGFTFSNYWMSWVRQSPEKGLEWVSEIRLRSDNYATHYAESVKGRFTISR
DNSKNTLYLQMNSLRAEDTGIYYCCTYFYSFSYWGQGTLVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSC
Heavy chain of HER2 targeting DVD-IgG1
(SEQ ID NO: 9)
(trastuzumab V_{H}/ASTKGP/h38C2_Cys V_{H}/C_{γ1}1-hinge_{γ1}-
C_{γ1}2-C_{γ1}3), minus the signal peptide The K99C
mutation (Kabat 93) is underlined and bolded.
(ASTKGP is SEQ ID NO: 5)
EVQLVESGGGLVQPGGSLRLSCAASGFNIKDTYIHWVRQAPGKGLEWVAR
IYPINGYTRYADSVKGRFTISADTSKNTAYLQMNSLRAEDTAVYYCSRWG
GDGFYAMDYWGQGTLVTVSSASTKGPEVQLVESGGGLVQPGGSLRLSCAA
SGFTFSNYWMSWVRQSPEKGLEWVSEIRLRSDNYATHYAESVKGRFTISR
DNSKNTLYLQMNSLRAEDTGIYYCCTYFYSFSYWGQGTLVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEV
KENWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVF
SCSVMHEALHNHYTQKSLSLSPGA
Light chain of HER2 targeting DVD-Fab and DVD-IgG1
(SEQ ID NO: 10)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKASTKGPELQMTQSPSSLSASVGDRVTITCRSSQSLLHTYGSPY
LNWYLQKPGQSPKLLIYKVSNRFSGVPSRFSGSGSGTDFTLTISSLQPED
FAVYFCSQGTHLPYTFGGGTKVEIKRTVAAPSVFIFPPSDEQLKSGTASV
VCLLNNFYPREAKVQWKVDNALQSGNSQESVTEQDSKDSTYSLSSTLTLS
KADYEKHKVYACEVTHQGLSSPVTKSENRGEC

The heavy chain amino acid sequence of the DVD-Fab molecule (trastuzumab V_{H}/ASTKGP/h38C2_Arg V_{H}/C_{γ1}1), minus the signal peptide, is shown in SEQ ID NO:11 (ASTKGP is SEQ ID NO:5). The heavy chain amino acid sequence of the exemplified DVD-Ig molecule (trastuzumab V_{H}/ASTKGP/h38C2_Arg V_{H}/C_{γ1}1-hinge_{γ1}-C_{γ1}2-C_{γ1}3), minus the signal peptide, is shown in SEQ ID NO: 12 (ASTKGP is SEQ ID NO:5). The linker sequence separating the two variable domains is underlined in these sequences. Constant region sequences are italicized in the sequences.

The heavy chain amino acid sequence of the DVD-Fab molecule (trastuzumab V_{H}/ASTKGP/h38C2_Lys V_{H}/C_{γ1}1), minus the signal peptide, is shown in SEQ ID NO: 13 (ASTKGP is SEQ ID NO:5). The heavy chain amino acid sequence of the exemplified DVD-Ig molecule (trastuzumab V_{H}/ASTKGP/h38C2_Lys V_{H}/C_{γ1}1-hinge_{γ1}-C_{γ1}2-C_{γ1}3), minus the signal peptide, is shown in SEQ ID NO:14 (ASTKGP is SEQ ID NO:5). The linker sequence separating the two variable domains is underlined in these sequences. Constant region sequences are italicized in the sequences.

Heavy chain of HER2 targeting DVD-Fab
(SEQ ID NO: 11)
(trastuzumab V_{H}/ASTKGP/h38C2_Arg V_{H}/C_{γ1}1),
minus the signal peptide The K99R mutation (Kabat
93) is bolded. (ASTKGP is SEQ ID NO:5)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPEVQLVESGGGLVQPGGSLRLSCAA
SGFTFSNYWMSWVRQSPEKGLEWVSEIRLRSDNYATHYAESVKGRFTISR
DNSKNTLYLQMNSLRAEDTGIYYCRTYFYSFSYWGQGTLVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSC
Heavy chain of HER2 targeting DVD-IgG1
(SEQ ID NO: 12)
(trastuzumab V_{H}/ASTKGP/h38C2_Arg V_{H}/C_{γ1}1-hinge_{γ1}-
C_{γ1}2-C_{γ1}3), minus the signal peptide The K99R
mutation (Kabat 93) is bolded. (ASTKGP is SEQ ID
NO: 5)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPEVQLVESGGGLVQPGGSLRLSCAA
SGFTFSNYWMSWVRQSPEKGLEWVSEIRLRSDNYATHYAESVKGRFTISR
DNSKNTLYLQMNSLRAEDTGIYYCRTYFYSFSYWGQGTLVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEV
KENWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVF
SCSVMHEALHNHYTQKSLSLSPGA
Heavy chain of HER2 targeting DVD-Fab
(SEQ ID NO: 13)
(trastuzumab V_{H}/ASTKGP/h38C2_Lys V_{H}/C_{γ1}1), minus
the signal peptide K99 (Kabat 93) is bolded.
(ASTKGP is SEQ ID NO: 5)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPEVQLVESGGGLVQPGGSLRLSCAA
SGFTFSNYWMSWVRQSPEKGLEWVSEIRLRSDNYATHYAESVKGRFTISR
DNSKNTLYLQMNSLRAEDTGIYYCKTYFYSFSYWGQGTLVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSC
Heavy chain of HER2 targeting DVD-IgG1
(SEQ ID NO: 14)
(trastuzumab V_{H}/ASTKGP/h38C2_Lys V_{H}/C_{γ1}1-hinge_{γ1}-
C_{γ1}2-C_{γ1}3), minus the signal peptide K99 (Kabat 93)
is bolded. (ASTKGP is SEQ ID NO: 5)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPEVQLVESGGGLVQPGGSLRLSCAA
SGFTFSNYWMSWVRQSPEKGLEWVSEIRLRSDNYATHYAESVKGRFTISR
DNSKNTLYLQMNSLRAEDTGIYYCKTYFYSFSYWGQGTLVTVSSASTKGP
SVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKT
HTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVF
SCSVMHEALHNHYTQKSLSLSPGA

In various embodiments, in addition to having Cys substitution for the reactive Lys residue in one or both arms of the 38C2 component, the HER2-targeting DVD compounds of the invention can contain a light chain amino acid sequence that is substantially similar to SEQ ID NO:10, for example, has at least about 80% amino acid sequence identity, alternatively has at least about 81%, 82%, 83%, 84%, 85%, 86%, 87%, 88%, 89%, 90%, 91%, 92%, 93%, 94%, 95%, 96%, 97%, 98%, or 99% amino acid sequence identity to SEQ ID NO: 10. Alternatively or additionally, the HER2-targeting DVD compounds can contain a heavy chain amino acid sequence that is substantially similar to SEQ ID NO:8 or 9, for example, has at least about 80% amino acid sequence identity, alternatively has at least about 81%, 82%, 83%, 84%, 85%, 86%, 87%, 88%, 89%, 90%, 91%, 92%, 93%, 94%, 95%, 96%, 97%, 98%, or 99% amino acid sequence identity to SEQ ID NO: 8 or 9.

As exemplification, the invention provides TVD-Ig and TVD-Fab containing 38C2_Cys for specifically targeting tumor antigen HER2. Some exemplary TVD compounds contain a variable domain that binds to HER2 and a humanized 38C2_Cys variable domain as the upper inner variable domain and a humanized 38C2_Cys variable domain as the lower inner variable domain. Some exemplary TVD compounds contain a variable domain that binds to HER2 and a humanized 38C2_Cys variable domain as the upper inner variable domain and a humanized 38C2_Lys variable domain as the lower inner variable domain. Some exemplary TVD compounds contain a variable domain that binds to HER2 and a humanized 38C2_Cys variable domain as the upper inner variable domain and a humanized 38C2_Arg variable domain as the lower inner variable domain. The variable domains can be connected on each light and heavy chain with a peptide linker sequence, e.g., ASTKGP (SEQ ID NO: 5). To facilitate recombinant protein production, a signal peptide sequence, e.g., MDWTWRILFLVAAATGAHS (SEQ ID NO:7), can be placed at the N-terminus of the heavy and light chain sequences. The light chain amino acid sequence of the TVD-Ig and TVD-Fab molecules exemplified herein, minus the signal peptide, is shown in SEQ ID NO: 30. The heavy chain amino acid sequence of the TVD-Fab molecule trastuzumab V_{H}/ASTKGP/h38C2_Cys V_{H}/ASTKGP/h38C2_Cys V_{H}/C_{γ1}1, minus the signal peptide, is shown in SEQ ID NO:24 (ASTKGP is SEQ ID NO:5). The heavy chain amino acid sequence of the exemplified TVD-Ig molecule trastuzumab V_{H}/ASTKGP/h38C2_Cys V_{H}/ASTKGP/h38C2_Cys V_{H}/C_{γ1}1-hinge_{γ1}-C_{γ1}2-C_{γ1}3, minus the signal peptide, is shown in SEQ ID NO:25 (ASTKGP is SEQ ID NO:5). The heavy chain amino acid sequence of the TVD-Fab molecule trastuzumab V_{H}/ASTKGP/h38C2_Cys V_{H}/ASTKGP/h38C2 V_{H}/C_{γ1}1, minus the signal peptide, is shown in SEQ ID NO:26 (ASTKGP is SEQ ID NO:5). The heavy chain amino acid sequence of the exemplified TVD-Ig molecule trastuzumab V_{H}/ASTKGP/h38C2_Cys V_{H}/ASTKGP/h38C2 V_{H}/C_{γ1}1-hinge_{γ1}-C_{γ1}2-C_{γ1}3, minus the signal peptide, is shown in SEQ ID NO:27 (ASTKGP is SEQ ID NO:5). The heavy chain amino acid sequence of the TVD-Fab molecule trastuzumab V_{H}/ASTKGP/h38C2_Cys V_{H}/ASTKGP/h38C2_Arg V_{H}/C_{γ1}1, minus the signal peptide, is shown in SEQ ID NO:28 (ASTKGP is SEQ ID NO:5). The heavy chain amino acid sequence of the exemplified TVD-Ig molecule trastuzumab V_{H}/ASTKGP/h38C2_Cys V_{H}/ASTKGP/h38C2_Arg V_{H}/C_{γ1}1-hinge_{γ1}-C_{γ1}2-C_{γ1}3, minus the signal peptide, is shown in SEQ ID NO:29 (ASTKGP is SEQ ID NO: 5). The linker sequences separating the three variable domains are underlined in these sequences. Constant region sequences are italicized in the sequences.

Heavy chain of HER2 targeting TVD-Fab
(SEQ ID NO: 24)
(trastuzumab V_{H}/ASTKGP/h38C2_Cys V_{H}/ASTKGP/h38C2_Cys V_{H}/C_{γ1}1), minus the
signal peptide The K99C mutation (Kabat 93) is underlined and bolded.
(ASTKGP is SEQ ID NO: 5)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPEVQLVESGGGLVQPGGSLRLSCAASGFTFSNYWMSWVRQS
PEKGLEWVSEIRLRSDNYATHYAESVKGRFTISRDNSKNTLYLQMNSLRAEDTGIYY
CCTYFYSFSYWGQGTLVTVSSASTKGPEVQLVESGGGLVQPGGSLRLSCAASGFTFS
NYWMSWVRQSPEKGLEWVSEIRLRSDNYATHYAESVKGRFTISRDNSKNTLYLQM
NSLRAEDTGIYYCCTYFYSFSYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHK
PSNTKVDKRVEPKSC
Heavy chain of HER2 targeting TVD-IgG1 (trastuzumab V_{H}/
(SEQ ID NO: 25)
ASTKGP/h38C2_Cys V_{H}/ASTKGP/h38C2_Cys V_{H}/C_{γ1}1-hinge_{γ1}-C_{γ1}2-C_{γ1}3), minus
the signal peptide The K99C mutation (Kabat 93) is underlined and bolded.
(ASTKGP is SEQ ID NO: 5)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPEVQLVESGGGLVQPGGSLRLSCAASGFTFSNYWMSWVRQS
PEKGLEWVSEIRLRSDNYATHYAESVKGRFTISRDNSKNTLYLQMNSLRAEDTGIYY
CCTYFYSFSYWGQGTLVTVSSASTKGPEVQLVESGGGLVQPGGSLRLSCAASGFTFS
NYWMSWVRQSPEKGLEWVSEIRLRSDNYATHYAESVKGRFTISRDNSKNTLYLQM
NSLRAEDTGIYYCCTYFYSFSYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHK
PSNTKVDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPG
A
Heavy chain of HER2 targeting TVD-Fab
(SEQ ID NO: 26)
(trastuzumab V_{H}/ASTKGP/h38C2_Cys V_{H}/ASTKGP/h38C2 V_{H}/C_{γ1}1), minus the signal
peptide The K99C mutation (Kabat 93) is bolded and underlined. K99 (Kabat
93) is bolded. (ASTKGP is SEQ ID NO: 5)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPEVQLVESGGGLVQPGGSLRLSCAASGFTFSNYWMSWVRQS
PEKGLEWVSEIRLRSDNYATHYAESVKGRFTISRDNSKNTLYLQMNSLRAEDTGIYY
CCTYFYSFSYWGQGTLVTVSSASTKGPEVQLVESGGGLVQPGGSLRLSCAASGFTFS
NYWMSWVRQSPEKGLEWVSEIRLRSDNYATHYAESVKGRFTISRDNSKNTLYLQM
NSLRAEDTGIYYCKTYFYSFSYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHK
PSNTKVDKRVEPKSC
Heavy chain of HER2 targeting TVD-IgG1
(SEQ ID NO: 27)
(trastuzumab V_{H}/ASTKGP/h38C2_Cys V_{H}/ASTKGP/h38C2 V_{H}/C_{γ1}1-hinge_{γ1}-C_{γ1}2-C_{γ1}3),
minus the signal peptide The K99C mutation (Kabat 93) is bolded and
underlined K99 (Kabat 93) is bolded. (ASTKGP is SEQ ID NO: 5)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPEVQLVESGGGLVQPGGSLRLSCAASGFTFSNYWMSWVRQS
PEKGLEWVSEIRLRSDNYATHYAESVKGRFTISRDNSKNTLYLQMNSLRAEDTGIYY
CCTYFYSFSYWGQGTLVTVSSASTKGPEVQLVESGGGLVQPGGSLRLSCAASGFTFS
NYWMSWVRQSPEKGLEWVSEIRLRSDNYATHYAESVKGRFTISRDNSKNTLYLQM
NSLRAEDTGIYYCKTYFYSFSYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHK
PSNTKVDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSN
KALPAPIEKTISKAKGQPREPQVYTIPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPG
A
Heavy chain of HER2 targeting TVD-Fab
(SEQ ID NO: 28)
(trastuzumab V_{H}/ASTKGP/h38C2_Cys V_{H}/ASTKGP/h38C2_Arg V_{H}/C_{γ1}1), minus
the signal peptide The K99C mutation (Kabat 93) is bolded and under-
lined. The K99R mutation (Kabat 93) is bolded. (ASTKGP is SEQ ID NO: 5)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPEVQLVESGGGLVQPGGSLRLSCAASGFTFSNYWMSWVRQS
PEKGLEWVSEIRLRSDNYATHYAESVKGRFTISRDNSKNTLYLQMNSLRAEDTGIYY
CCTYFYSFSYWGQGTLVTVSSASTKGPEVQLVESGGGLVQPGGSLRLSCAASGFTFS
NYWMSWVRQSPEKGLEWVSEIRLRSDNYATHYAESVKGRFTISRDNSKNTLYLQM
NSLRAEDTGIYYCRTYFYSFSYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHK
PSNTKVDKRVEPKSC
Heavy chain of HER2 targeting TVD-IgG1
(SEQ ID NO: 29)
(trastuzumab VH/ASTKGP/h38C2_Cys V_{H}/ASTKGP/h38C2_Arg V_{H}/C_{γ1}1-hinge_{γ1}-
C_{γ1}2-C_{γ1}3), minus the signal peptide The K99C mutation (Kabat 93) is
bolded and underlined. The K99R mutation (Kabat 93) is bolded.
(ASTKGP is SEQ ID NO: 5)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPEVQLVESGGGLVQPGGSLRLSCAASGFTFSNYWMSWVRQS
PEKGLEWVSEIRLRSDNYATHYAESVKGRFTISRDNSKNTLYLQMNSLRAEDTGIYY
CCTYFYSFSYWGQGTLVTVSSASTKGPEVQLVESGGGLVQPGGSLRLSCAASGFTFS
NYWMSWVRQSPEKGLEWVSEIRLRSDNYATHYAESVKGRFTISRDNSKNTLYLQM
NSLRAEDTGIYYCRTYFYSFSYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHK
PSNTKVDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPG
A
Light chain of HER2 targeting TVD-Fab and TVD-IgG1
(SEQ ID NO: 30)
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKASTKGPELQ
MTQSPSSLSASVGDRVTITCRSSQSLLHTYGSPYLNWYLQKPGQSPKLLIYKVSNRFS
GVPSRFSGSGSGTDFTLTISSLQPEDFAVYFCSQGTHLPYTFGGGTKVEIKASTKGPEL
QMTQSPSSLSASVGDRVTITCRSSQSLLHTYGSPYLNWYLQKPGQSPKLLIYKVSNRF
SGVPSRFSGSGSGTDFTLTISSLQPEDFAVYFCSQGTHLPYTFGGGTKVEIKRTVAAPSV
FIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

In various embodiments, in addition to having Cys substitution for the reactive Lys residue in one or both arms of the 38C2 component, the HER2-targeting TVD compounds of the invention can contain a light chain amino acid sequence that is substantially similar to SEQ ID NO:30, for example, has at least about 80% amino acid sequence identity, alternatively has at least about 81%, 82%, 83%, 84%, 85%, 86%, 87%, 88%, 89%, 90%, 91%, 92%, 93%, 94%, 95%, 96%, 97%, 98%, or 99% amino acid sequence identity to SEQ ID NO:30. Alternatively or additionally, the HER2-targeting TVD compounds can contain a heavy chain amino acid sequence that is substantially similar to any one of SEQ ID NOs: 24-29, for example, has at least about 80% amino acid sequence identity, alternatively has at least about 81%, 82%, 83%, 84%, 85%, 86%, 87%, 88%, 89%, 90%, 91%, 92%, 93%, 94%, 95%, 96%, 97%, 98%, or 99% amino acid sequence identity to any one of SEQ ID NOs: 24-29.

### V. Linker Moieties for Conjugating Drugs

The drug moieties in the antibody conjugate drugs (ADCs) of the invention are typically conjugated in a site-specific manner to the 38C2_Cys antibody via an appropriate linker sequence or linker moiety. The linkers serve to attach the cargo moiety (e.g., a drug moiety) to the DVD-Ig or TVD-Ig, and can employ any suitable chemistry. Various types of linker functionality can be included in the ADCs of the invention, including but not limited to cleavable linkers, and non-cleavable linkers, as well as reversible linkers and irreversible linkers.

Cleavable linkers are those that rely on processes inside a target cell to liberate a drug moiety, such as reduction in the cytoplasm, exposure to acidic conditions in a lysosome or endosome, or cleavage by specific enzymes (e.g. proteases) within the cell. As such, cleavable linkers allow an attached drug moiety to be released in its original form after an immunoconjugate has been internalized and processed inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates). Non-cleavable linkers utilize catabolic degradation of an immunoconjugate for the release of the drug moiety. A released drug moiety generally retains the linker as well as the amino acid residue of the immunoglobulin to which the linker was conjugated. Non-cleavable linkers include, but are not limited to, PEG linkers, hydrocarbon linkers, and thioether linkers.

Reversible linkers utilize chemical bonds that can readily be broken, or reversed, using suitable reagents. As such, after the formation of a reversible linker, the linker can be broken in a desired position by treatment with a reagent, thereby releasing the immunoglobulin molecule from the linker. Irreversible linkers utilize chemical bonds that cannot readily be broken or reversed after their formation. As such, after the formation of an irreversible linker, an immunoglobulin molecule cannot readily be released.

For site-specific conjugation to the reactive Cys residue in the ADCs of the invention, any chemical moieties known in the art that are reactive with the residues may be employed. For example, non-limiting examples of suitable linkers include, e.g., maleimide, monobromomaleimide, or dibromomaleimide. As noted above, some ADCs of the invention also contain drug moieties that are conjugated to the reactive lysine residue in 38C2 or to an engineered arginine residue in 38C2 in addition to the engineered cysteine residues. Various other linker moieties can be used for the site-specific Lys conjugation. See, e.g., WO2017/049139. For example, non-limiting examples of reversible linkers for site-specific lysine conjugation include, for example, diketone moieties. Non-limiting examples of irreversible linkers for site-specific lysine conjugation include, for example, β-lactam moieties. For site-specific conjugation to the reactive Arg residue in the ADCs of the invention, any chemical moieties known in the art that are reactive with the residues may be employed. For example, non-limiting examples of suitable linkers include, e.g., of phenylglyoxal (PGO), glyoxal (GO), and methylglyoxal (MGO). See, e.g., Takahashi, J. Biochem. 81:395-402, 1977.

Some linkers of the invention have the structure as depicted below:

electrophilic-aromatic-spacer of sufficient length to reach reactive residue 99 at bottom of hydrophobic pocket

In some embodiments, the linker for attaching the drug moieties can contain an amino acid unit. The amino acid unit allows for cleavage of the linker by a protease, thereby facilitating release of the drug from the immunoconjugate upon exposure to intracellular proteases, such as lysosomal enzymes. See, e.g., Doronina et al. (2003) Nat. Biotechnol. 21:778-784. Non-limiting examples of amino acid units include, but are not limited to, a dipeptide, a tripeptide, a tetrapeptide, and a pentapeptide. Non-limiting examples of dipeptides include: valine-citrulline (vc or val-cit), alanine-phenylalanine (af or ala-phe); phenylalanine-lysine (fk or phe-lys); or N-methyl-valine-citrulline (Me-val-cit). Non-limiting examples of tripeptides include: glycine-valine-citrulline (gly-val-cit) and glycine-glycine-glycine (gly-gly-gly). An amino acid unit can comprise amino acid residues that occur naturally, as well as minor amino acids and non-naturally occurring amino acid analogs, such as citrulline. Amino acid units can be designed and optimized in their selectivity for enzymatic cleavage by a particular enzyme, for example, a tumor-associated protease, cathepsin B, C and D, or a plasmin protease.

In some embodiments, the linker can be a branched or dendritic type linker moiety for covalent attachment of more than one drug moiety through a branching, multifunctional linker moiety to an immunoglobulin (Sun et al (2002) Bioorganic & Medicinal Chemistry Letters 12:2213-2215; Sun et al (2003) Bioorganic & Medicinal Chemistry 11:1761-1768). Non-limiting examples of branched, dendritic linkers include 2,6-bis(hydroxymethyl)-p-cresol and 2,4,6-tris(hydroxymethyl)-phenol dendrimer units (WO 2004/01993; Szalai et al (2003) J. Amer. Chem. Soc. 125:15688-15689; Shamis et al (2004) J. Amer. Chem. Soc. 126:1726-1731; Amir et al (2003) Angew. Chem. Int. Ed. 42:4494-4499). Branched linkers can increase the molar ratio of drug to immunoglobulin, i.e., loading, which is related to the potency of the ADC. Thus, for example, where an immunoglobulin bears only one reactive amino acid residue for conjugation, a multitude of drug moieties can be attached through a branched linker.

The linkers suitable for use in the ADCs of the invention, including stretcher, spacer, and amino acid units, can be synthesized by methods known in the art, such as those described in US Patent Publication No. 2005/0238649 A1.

### VI. Payloads or Cargo Moieties

The ADCs of the invention are intended to deliver a payload or cargo moiety (e.g., a drug) to the specific target of interest. The payload broadly includes, but are not limited to, biologically active moieties, such as drug moieties and expression modifying moieties, as well as non-biologically active moieties, such as detectable moieties (e.g., detectable labels). Non-limiting examples of drug moieties include cytotoxic and cytostatic agents that are capable of killing a target cell, or arresting the growth of a target cell. In some embodiments, the employed drug moieties are toxins, chemotherapeutic agents, antibiotics, radioactive isotopes, chelated radioactive isotopes, and nucleolytic enzymes. In some embodiments, the drug moieties for the ADCs of the invention can be polymerized drugs that consist of a polymer drugs. For example, the payload in the ADCs can be polymerized drugs generated via the Fleximer technology developed by Mersana Therapeutics (Cambridge, MA). See, e.g., Yurkovetskiy et al., Cancer Res. 2015, 75:3365-72.

In some embodiments, the payload in the ADCs of the invention is a drug moiety selected from the group consisting of auristatin; dolostatin; cemadotin; amanitin (including but not limited to α-amanitin); monomethyl auristatin F (MMAF); monomethyl auristatin E (MMAE); maytansinoids (including, but not limited to DM1, DM3 and DM4); pyrrolobenzodiazepines (PBDs, including, but not limited to monomeric and dimeric PBDs); indolinobenzodiazepine (including, but not limited to dimeric indolinobenzodiazepines); enediynes (including but not limited to calicheamicins and tiancimycins); camptothecins (including but not limited to SN-38); doxorubicin (including but not limited to MMDX or bioactivation products thereof, such as, e.g., PNU-159682); a duocarmycin; a cepafungin. In some embodiments, the drug moiety in the ADCs of the invention is selected from a group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, a proteasome inhibitor, an inhibitor of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, or a molecular glue agent for proximity-induced targeted protein degradation (as described, e.g., in Dong et al., J. Med. Chem. 64, 10606-10620, 2021).

In some embodiments, the ADCs of the invention can contain a drug moiety that modifies a given biological response. Drug moieties are not to be construed as limited to classical chemical therapeutic agents. For example, a drug moiety can be a protein, peptide, or polypeptide possessing a desired biological activity. Such proteins can include, for example, a toxin such as abrin, ricin A, pseudomonas exotoxin, cholera toxin, or diphtheria toxin, a protein such as tumor necrosis factor, α-interferon, β-interferon, nerve growth factor, platelet derived growth factor, tissue plasminogen activator, a cytokine, an apoptotic agent, an anti-angiogenic agent, or a biological response modifier such as, for example, a lymphokine. In some embodiments, the drug moiety can be a cytotoxin, a drug (e.g., an immunosuppressant) or a radiotoxin. Examples of cytotoxins include but are not limited to, taxanes, DNA-alkylating agents (e.g., CC-1065 analogs), anthracyclines, tubulysin analogs, duocarmycin analogs, auristatin E, auristatin F, maytansinoids, and cytotoxic agents comprising a reactive polyethylene glycol moiety, taxon, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Drug moieties can also include, for example, anti-metabolites (e.g., methotrexate, 6-mercaptopurine, 6-thioguanine, cytarabine, 5-fluorouracil decarbazine), ablating agents (e.g., mechlorethamine, thiotepa chlorambucil, meiphalan, carmustine (BSNU) and lomustine (CCNU), cyclophosphamide, busulfan, dibromomannitol, streptozotocin, mitomycin C, and cis-dichlorodiamine platinum (II) (DDP) cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). See, e.g., US Patent Publication No. 20090304721, which is incorporated herein by reference in its entirety. Other non-limiting examples of cytotoxins that can be conjugated to the antibodies, antibody fragments (antigen or hapten binding fragments) or functional equivalents of the invention include duocarmycins, calicheamicins, maytansines and auristatins, and derivatives thereof.

The cargo moieties in the ADCs of the invention can also be a radioactive isotope or a chelated radioactive isotope to generate cytotoxic radiopharmaceuticals, referred to as radioimmunoconjugates. Examples of radioactive isotopes that can be conjugated to antibodies for use diagnostically or therapeutically include, but are not limited to, iodine-131, indium-111, yttrium-90, lutetium-177, bismuth-213 and astatine-211. Methods for preparing radioimmunoconjugates are established in the art. Examples of radioimmunoconjugates are Zevalin™ (IDEC Pharmaceuticals) and Bexxar™ (Corixa Pharmaceuticals), and similar methods can be used to prepare radioimmunoconjugates using the antibodies of the invention. In some embodiments, the macrocyclic chelator is 1,4,7,10-tetraazacyclododecane-N,N′,N″,N′″-tetraacetic acid (DOTA) or isomers (CHX-A′, CHA-A″, CHX-B′ and CHX-B″) of 2-(p-isothiocyanatobenzyl)-cyclohexyl-diethylenetriaminepentaacetic acid (CHX-DTPA), which can be attached to an immunoglobulin via a linker molecule.

In some embodiments, the payload of the ADCs of the invention can be a photoabsorber for near infrared (NIR) photoimmunotherapy (PIT). PIT is a tumor-targeted anticancer platform that can induce a rapid and specific destruction of the tumor. The treatments consist of a drug (a cancer-targeting photoactivatable antibody conjugate) and a device system to apply light at the tumor site. PIT is unique in that it combines molecular targeting of the cancer cells to achieve high tumor specificity, together with a biophysical mechanism of cancer cell destruction that results in broad spectrum anticancer activity regardless of the tumorigenic mechanism of the patients' tumor. See, e.g., Mitsunaga et al., Nat. Med. 17:1685-92, 2011. For example, the DVD or TVD compounds of the invention can include a NIR PIT photoabsorber (e.g., IR700) and an antigen-binding variable domain region targeting tumor cells.

In various embodiments, the payload of the ADCs of the invention can be a single drug unit or a plurality of identical drug units, such as 2, 3, 4, 5, 6, 7, 8, 9, or 10 drug units on the same drug moiety. In some embodiments, the drug moiety includes two different drug units on the same drug moiety. For example, in some aspects, a single drug moiety can include both an MMAF drug unit and a PBD monomer drug unit. Furthermore, in certain aspects, a subject immunoconjugate can include a first drug moiety conjugated to a first arm of the immunoconjugate, and a second drug moiety conjugated to the second arm of the immunoconjugate. As such, any of a variety of combinations of drug moieties can be conjugated to a subject DVD-Ig or TVD-Ig via a linker. As exemplification, the ADCs can contain a site-specific Cys conjugated carboxytetramethylrhodamine (TAMRA) and a site-specific Lys conjugated MMAF on its two arms.

In some embodiments, the cargo moieties in the ADCs of the invention are expression modifying moieties. Expression modifying moieties include, but are not limited to, non-protein-coding RNA (“npcRNA”). In some embodiments, the npcRNA can be, e.g., a microRNA (miRNA), a miRNA precursor, a small interfering RNA (siRNA), a small RNA and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a tracer RNA (tcRNA), a guide RNA (gRNA), and a single-guide RNA (sgRNA).

In some embodiments, the cargo moieties in the ADCs of the invention are detectable moieties. Detectable moieties include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives including carboxytetramethylrhodamine (TAMRA), dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

In some embodiments, the cargo moieties in the ADCs of the invention are small molecule-based proteolysis targeting chimeras (PROTAC) (see, e.g., Y. Zou et al., Cell Biochem Funct. 2019 January; 37(1): 21-30; X. Li and Y. Song, J Hematol Oncol. 2020 May 13; 13(1):50; S.-M. Qi et al., Front Pharmacol 2021 May 7; 12:692574).

### VII. Production of Site-Specific Arg Conjugated ADCs

The site-specific cysteine conjugated antibody conjugate drugs (ADCs) of the invention can be produced with any methods known in the art and the specific techniques exemplified herein. For example, expression from host cells, wherein expression vector(s) encoding the DVD or TVD heavy and/or DVD or TVD light chains is transfected into a host cell by standard techniques. Various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the DVD or TVD immunoglobulins of the invention in either prokaryotic or eukaryotic host cells, expression of DVD or TVD immunoglobulins in eukaryotic cells is preferable, and most preferable in mammalian host cells, because such eukaryotic cells (and in particular mammalian cells) are more likely than prokaryotic cells to assemble and secrete a properly folded and immunologically active DVD or TVD immunoglobulin.

Preferred mammalian host cells for expressing the recombinant immunoglobulins of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), Human Embryonic Kidney (HEK) cells, NSO myeloma cells, COS cells and SP2 cells. When recombinant expression vectors encoding DVD or TVD immunoglobulins are introduced into mammalian host cells, the DVD or TVD immunoglobulins are produced by culturing the host cells for a period of time sufficient to allow for expression of the DVD or TVD immunoglobulins in the host cells or, more preferably, secretion of the DVD or TVD immunoglobulins into the culture medium in which the host cells are grown. DVD or TVD immunoglobulins can be recovered from the culture medium using standard protein purification methods.

In a preferred system for recombinant expression of DVD or TVD immunoglobulins of the invention, a recombinant expression vector encoding both the DVD or TVD heavy chain and the DVD or TVD light chain is introduced into dhfr-CHO cells by calcium phosphate-mediated transfection. Within the recombinant expression vector, the DVD or TVD heavy and light chain genes are each operatively linked to CMV enhancer/AdMLP promoter regulatory elements to drive high levels of transcription of the genes. A recombinant expression vector also carries a DHFR gene, which allows for selection of CHO cells that have been transfected with the vector using methotrexate selection/amplification. Selected transformant host cells are cultured to allow for expression of the DVD or TVD heavy and light chains and intact DVD or TVD immunoglobulin is recovered from the culture medium. Standard molecular biology and tissue culture techniques are used to prepare the recombinant expression vector, transfect the host cells, select for transformants, culture the host cells and recover the DVD or TVD immunoglobulin from the culture medium. In addition, aspects of the invention include a method of synthesizing a DVD or TVD immunoglobulin of the invention by culturing a host cell of the invention in a suitable culture medium until a DVD or TVD immunoglobulin of the invention is synthesized. A method can further comprise isolating the DVD or TVD immunoglobulin from the culture medium to yield an isolated immunoglobulin.

A feature of the subject DVD or TVD immunoglobulins is that they can be produced and purified in ways that are similar to conventional antibodies. Production of DVD or TVD immunoglobulins can result in a homogeneous, single major product with desired activity, without any sequence modification of the constant region or chemical modifications of any kind.

### VIII. Therapeutic Applications and Pharmaceutical Combinations

The site-specific Cys conjugated ADCs of the invention can be used in a variety of prophylactic, therapeutic and diagnostic applications. The specific application of an ADC of the invention will depend on the payload or drug moiety conjugated to the antibody compound. When a DVD or TVD based ADC is used, the specific application is also depending on the target molecule that is recognized by the second variable domain in the DVD or the third variable domain in the TVD. Thus, the antibody compounds and ADCs described herein can be employed in the treatment of various tumors. The compounds of the invention can be readily applied in many specific cancer therapies. Such therapeutic applications include, e.g., delivery of drug moieties to tumors via a known tumor targeting antibody or antigen-binding variable domain as exemplified herein. They also include treatments not directly targeting tumor cells, e.g., antibody-siRNA conjugates for targeting T cells, other immune cells, and tumor-supporting cells. They further include other non-conventional cancer therapies, e.g., the use of near infrared (NIR) photoimmunotherapy (PIT) for treating tumors (as well as non-tumor cells). The compounds of the invention (e.g., DVD or TVD based ADCs) can also be used in treating non-oncology indications such as infectious diseases, autoimmune diseases, cardiovascular diseases, metabolic diseases. See, e.g., Beck et al., Nat Rev Drug Discov. 2017, 16:315-337.

As exemplification, some ADCs of the invention including ADCs containing the HER2-targeting DVD compounds exemplified herein, can be used in the treatment of various cancers and other diseases by targeting and killing cells that express a particular tumor antigen. Suitable types of cancers include, without limitation, hematologic cancers, carcinomas, sarcomas, melanoma, and central nervous system cancers. Non-limiting examples of hematologic cancers that can be treated with the ADCs of the invention include leukemia, acute myeloid leukemia, acute lymphoblastic leukemia, chronic myelogenous leukemia, chronic lymphocytic leukemia, lymphoma, Hodgkin's lymphoma, non-Hodgkin's lymphoma, myeloma and myelodysplastic syndrome. Non-limiting examples of carcinomas that can be treated with the ADCs of the invention include skin cancer, head and neck, thyroid, lung, nasopharyngeal, colorectal, liver, urinary bladder, ovarian, cervical, endometrial, prostate, gastric, esophageal, pancreatic, renal, and breast cancer. Non-limiting examples of sarcomas that can be treated with the ADCs of the invention include angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, Kaposi's sarcoma and synovial sarcoma. Non-limiting examples of central nervous system cancers that can be treated with the ADCs of the invention include glioma, meningioma and neuroma. Non-limiting examples of other cancers that can be treated with the ADCs of the invention include melanoma.

In some embodiments, the ADCs of the invention can be used in conjunction with one or more additional therapies to treat a particular cancer. For example, the ADCs of the invention can be used in combination with or as an adjunct to conventional treatment with other medications such as an anti-neoplastic agent, a cytotoxic agent, an anti-angiogenic agent, or an immunosuppressive agent. Non-limiting examples of additional therapeutic agents include cisplatin, carboplatin, oxaliplatin, mechlorethamine, cyclophosphamide, chlorambucil, ifosfamide, doxorubicin, daunorubicin, valrubicin, idarubicin, epirubicin, actinomycin, bleomycin, plicamycin, mitomycin, bevacizumab, imatinib, erlotinib, gefitinib, ibrutinib, idelalisib, lenalidomide, vincristine, vinblastine, vinorelbine, vindesine, paclitaxel, and docetaxel. Any anti-neoplastic agents can be used in such a combination therapy. These include conventional and/or experimental chemotherapeutic agents, radiation treatments, and the like.

For therapeutic uses, the ADCs of the invention can be formulated into pharmaceutical compositions. The pharmaceutical compositions typically contain an effective amount of an immunoconjugate and a pharmaceutically acceptable carrier. The pharmaceutical composition of the present invention can be administered by a variety of methods known in the art. As will be appreciated by the skilled artisan, the route and/or mode of administration will vary depending upon the target disease or condition and the desired results. To administer a compound of the invention by certain routes of administration, it can be necessary to coat the compound with, or co-administer the compound with, a material to prevent its inactivation. For example, a compound can be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

Pharmaceutical compositions of the invention can also contain adjuvants such as preservatives, wetting agents, emulsifying agents and/or dispersing agents. Prevention of the presence of microorganisms can be ensured both by sterilization procedures and by the inclusion of various antibacterial and antifungal agents, for example, paraben, chlorobutanol, phenol, sorbic acid, and the like. It can also be desirable to include isotonic agents, such as sugars, sodium chloride, and the like into the compositions. In addition, prolonged absorption of the injectable pharmaceutical form can be brought about by the inclusion of agents that delay absorption, such as aluminum monostearate and gelatin.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. A selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical fields. The pharmaceutical compositions must be sterile and fluid to the extent that the composition is deliverable by syringe. In addition to water, the carrier preferably is an isotonic buffered saline solution.

Pharmaceutical compositions of the invention can further contain an effective amount of a second therapeutic agent. In some embodiments, the second therapeutic agent is an antibody, an anti-neoplastic agent, a cytotoxic agent, an anti-angiogenic agent, or an immunosuppressive agent. In some embodiments, the second therapeutic agent is selected from the group consisting of: cisplatin, carboplatin, oxaliplatin, mechlorethamine, cyclophosphamide, chlorambucil, ifosfamide, doxorubicin, daunorubicin, valrubicin, idarubicin, epirubicin, actinomycin, bleomycin, plicamycin, mitomycin, bevacizumab, imatinib, erlotinib, gefitinib, ibrutinib, idelalisib, lenalidomide, vincristine, vinblastine, vinorelbine, vindesine, paclitaxel, and docetaxel.

The following examples, sequences and figures are provided to aid the understanding of the present invention, the true scope of which is set forth in the appended claims. It is understood that modifications can be made in the procedures set forth without departing from the spirit of the invention.

### EXAMPLES

The following examples are offered to illustrate, but not to limit the present invention.

### Example 1: Generation, Characterization, and Crystallization of h38C2_K99C Fab and DVD-Fab

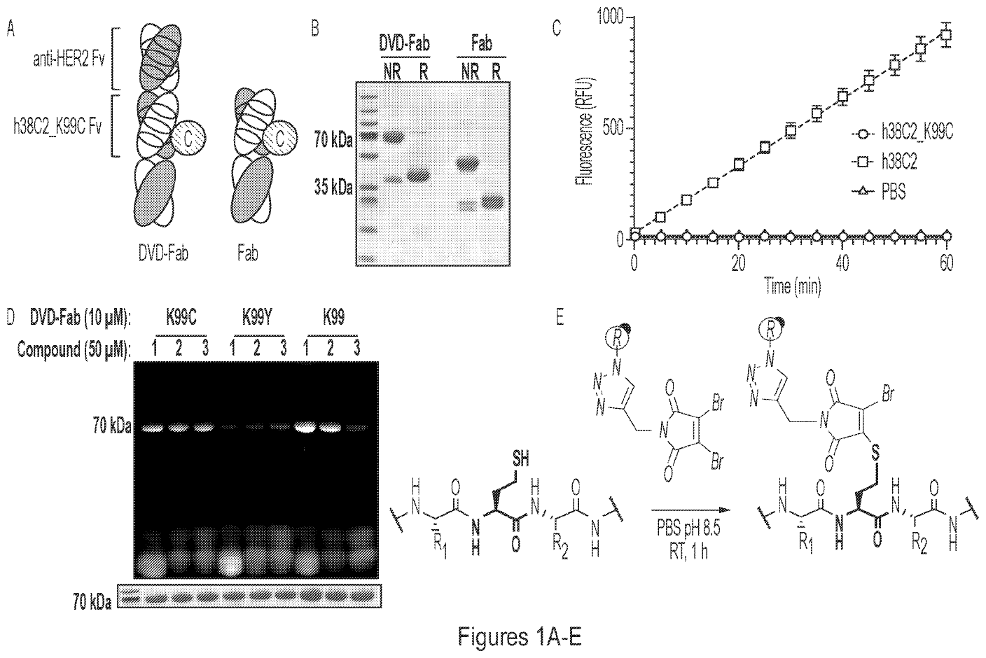

The reactive lysine residue of humanized catalytic antibody h38C2 is located at position 99 of the variable heavy chain domain which corresponds to position 93 by Kabat numbering [17]. The mutated h38C2_K99C in Fab and DVD-Fab format were cloned, expressed, and purified as previously described for h38C2_K99R Fab and DVD-Fab [14]. In the DVD-Fab, the outer Fv was derived from humanized anti-HER2 mAb trastuzumab and paired with h38C2_K99C as inner Fv (FIG. 1A). Analysis by nonreducing and reducing SDS-PAGE and Coomassie staining revealed the correct bands (FIG. 1B). As expected, the K99C mutation led to a complete loss of h38C2's catalytic activity as measured by a retro-aldol reaction that produces a fluorescent aldehyde (FIG. 1C).

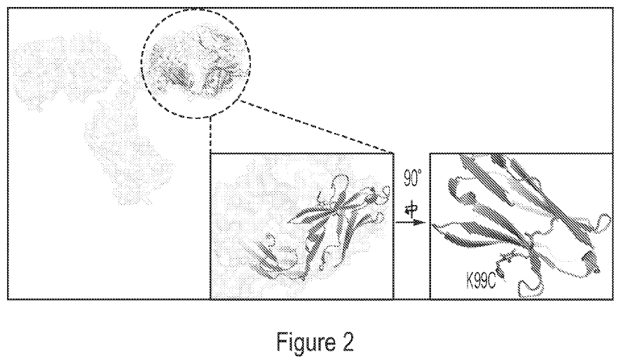

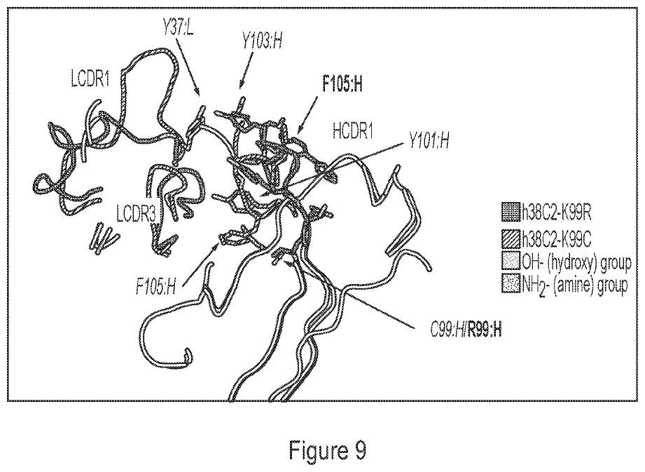

To confirm the placement of an unpaired cysteine residue at the bottom of a deep pocket, we next determined the three-dimensional structure of h38C2_K99C Fab by X-ray crystallography (FIG. 2). The best crystal of h38C2_K99C Fab diffracted to 2.4 Å Bragg spacings in a P2₁ space group with 4 independent molecules in the asymmetric unit (Table 3). While the variable light (V_{L}) and heavy chain (V_{H}) domains of all molecules in the asymmetric unit had good electron density, there were regions in the constant light (C_{L}) and heavy chain (C_{H}1) domains where poor electron density made modeling and refinement of the Fab difficult. The average Ca RMSD of the independent molecules were 0.50 Å. All aa residues constituting the active site, an 11-Å deep hydrophobic pocket at the interface of V_{H} and V_{L}, overlayed nicely. The thiol group of the mutated aa residue, C99 was positioned at the bottom of the pocket. Importantly, it did not interfere with the conserved intradomain disulfide bridge of V_{H} formed by C98 and C22. The Cα RMSD between the Fv of h38C2_K99C and the Fv of h38C2_K99R (PDBID 6U85; [14]) was 0.53 Å. However, the two structures diverged in three variable loops, namely, HCDR3, LCDR1, and LCDR3 (FIG. 9). The structural alteration appears to be propagated from the mutated cysteine residue which has a shorter side chain than lysine or arginine. The pocket region the guanidine group of the engineered arginine in h38C2_K99R occupies is replaced with the phenyl group of a phenylalanine (F105) in the HCDR3 of h38C2_K99C. This is caused by a 180° rotation of F105 and an inward shifting of HCDR3. Y101 of the HCDR3 forms a π-π interaction with F105 and further crowds the pocket. The intrusion of Y101 pushes the LCDR3 3 Å away from the center of the pocket. Interestingly, the shifted LCDR3 conformation overlaps well with that of the Fv of 33F12 in complex with its 1,3-diketone hapten (PDB ID 3F09; [12]). (V_{L} and V_{H} of 33F12 and 38C2 share 91% and 94% aa sequence identity, respectively). LCDR1 of h38C2_K99C is not involved in any significant crystal packing interaction. The 4 LCDR1s of the asymmetric unit of the h38C2_K99C crystal show an extended conformation compared to a compact helical structure in the h38C2_K99R crystal. Constructive interactions that might promote the extended conformation are a π-π interaction between Y37 of LCDR1 and Y103 of HCDR3 and an extended beta-sheet mediated by a hydrogen bond between Y37 of LCDR1 and framework residue G96 of LCDR3.

TABLE 3
Data collection and refinement statistics

| Protein (PDB ID) | 7TUS | |
|---|---|---|
| Wavelength (Å) | 1.12723 | |
| Space group | P 2₁ | |
| Unit cell a, b, c (Å) | 117.47, 73.57, 127.58 | |
| Unit cell alpha, beta, gamma (°) | 90, 110.06, 90 | |
| Asymmetric unit | 4 molecules | |
| Resolution (Å) ¹ | 39.95-2.4 | (2.5-2.4) |
| Unique reflections ¹ | 78594 | (4513) |
| Mean I/σI ¹ | 9.5 | (1.9) |
| Completeness (%) ¹ | 98.0 | (98.5) |
| Multiplicity ¹ | 3.4 | (3.5) |
| R_{merge} ¹ | 0.076 | (0.683) |
| CC½ ¹ | 0.995 | (0.692) |
| Wilson B-factor (Å²) | 43.26 | |
| Resolution (Å) ¹ | 39.95-2.4 | (2.49-2.4) |
| No. of reflections ¹ | 78534 | (7846) |
| No. of reflections in R_{free} ^{1, 2} | 3770 | (411) |
| R_{work} ¹ | 0.2162 | (0.3383) |
| R_{free} ^{1, 2} | 0.2552 | (0.3656) |

| | |
|---|---|
| R.m.s. Bond length (Å) | 0.014 |
| R.m.s. Bond angle (°) | 1.29 |
| B-factor, average (Å²) | 74.35 |
| B-factor, Ligand (Å²) | 75.01 |
| B-factor, Water (Å²) | 48.86 |
| Protein | 2386 |
| Water | 426 |
| Ramachandran favored | 96.05 |
| Ramachandran allowed | 3.83 |
| Ramachandran outliers | 0.12 |
| Rotamer outliers | 2.15 |

¹ Parentheses refer to statistics for the highest resolution shell.
²R_{free} is calculated with removal of 2% of the reflections as the test set before the refinement.

### Example 2: Survey of a Suitable Bioconjugation for h38C2_K99C DVD-Fab

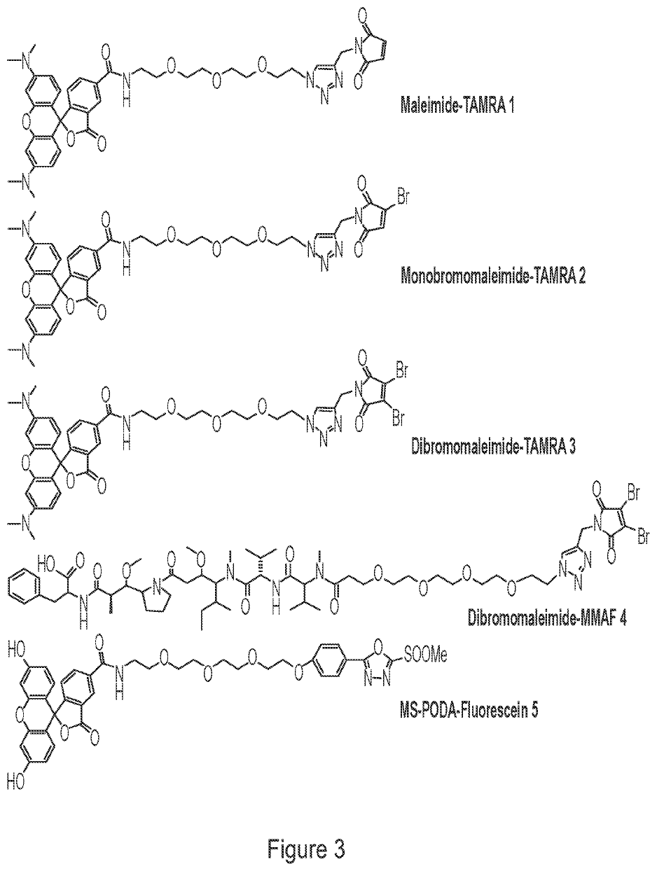

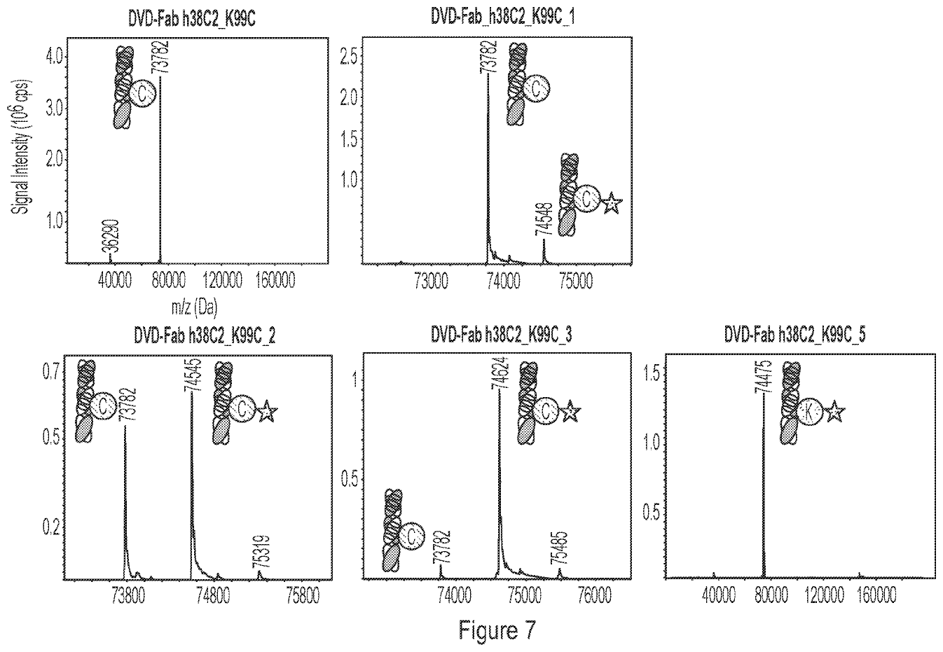

To investigate the reactivity and selectivity of the engineered cysteine toward maleimide derivatives, we synthesized tetramethylrhodamine (TAMRA) derivatives with maleimide (1), monobromomaleimide (2), and dibromomaleimide (3) functionality (FIG. 3). Bioconjugation to h38C2_K99C DVD-Fab was compared to parental h38C2 DVD-Fab as well as h38C2_K99Y DVD-Fab, which was cloned, expressed, and purified analogous to h38C2_K99C DVD-Fab. Using a molar ratio of 1:5, the 3 proteins were incubated with the 3 compounds in 9 separate reactions for 1 h at room temperature (RT) in PBS pH 8.5. Note the omission of a reducing reagent commonly used for bioconjugation to the thiol group of natural and engineered cysteine residues. Following bioconjugation, analysis by nonreducing SDS-PAGE and blue light visualization revealed a strong fluorescent band of ˜70 kDa for the bioconjugation of compounds 1 and 2 to h38C2_K99C DVD-Fab and parental h38C2 DVD-Fab but not h38C2_K99Y DVD-Fab (FIG. 1D). This result indicated that maleimide and monobromomaleimide can react with both uniquely reactive lysine and cysteine residues in the deep pocket of h38C2. Interestingly, bioconjugation of compound 3 was only observed for h38C2_K99C DVD-Fab, suggesting that dibromomaleimide selectively reacts with the engineered cysteine residue (FIG. 1D). Following this initial qualitative analysis, the efficiency of bioconjugating compounds 1, 2, and 3 to h38C2_K99C DVD-Fab was assessed by mass spectrometry. Compound 5, a fluorescein derivative with methylsulfone phenyloxadiazole (MS-PODA) functionality (FIG. 3), was included. MS-PODA::cysteine conjugates are more stable than maleimide::cysteine conjugates (thiosuccinimides) which readily hydrolyze through a retro-Michael reaction [18]. We showed recently that derivatives with MS-PODA functionality can also be rapidly, efficiently, and selectively conjugated to the reactive lysine residue of h38C2 [12]. The molecular weight of the unconjugated h38C2_K99C DVD-Fab was measured as 73,782 Da (expected 73,776.5 Da), and the conjugated DVD-Fabs h38C2_K99C_2, h38C2_K99C_3, and h38C2_K99C_5 gave major peaks at 74,545 Da (55%), 74,625 Da (90%), and 74,475 Da (55%), respectively, revealing a single conjugation event (FIG. 7). Notably, the difference in molecular weight between DVD-Fabs h38C2_K99C_2 (monobromomaleimide) and h38C2_K99C_3 (dibromomaleimide) was 80 Da which corresponds to one bromine atom (79.9 Da). Their molecular weight further suggested that both the monobromomaleimide::cysteine conjugation of 2 and the dibromomaleimide::cysteine conjugation of 3 proceeded by substituting one bromine to afford the thioether of the thio-monobromomaleimide (FIG. 1E). Although compound 1 revealed a similar bioconjugation efficiency to h38C2_K99C DVD-Fab as compounds 2 and 3 by nonreducing SDS-PAGE and blue light visualization (FIG. 1D), mass spectrometry revealed the unconjugated antibody as the major peak of this reaction (FIG. 7). Collectively, dibromomaleimide emerged as a superior electrophile for efficient site-specific bioconjugation to the buried cysteine residue of h38C2_K99C DVD-Fab.

### Example 3: Survey of a Suitable Bioconjugation for h38C2_K99C DVD-Fab

To investigate the reactivity and selectivity of the engineered cysteine toward maleimide derivatives, we synthesized tetramethylrhodamine (TAMRA) derivatives with maleimide (1), monobromomaleimide (2), and dibromomaleimide (3) functionality (FIG. 3). Bioconjugation to h38C2_K99C DVD-Fab was compared to parental h38C2 DVD-Fab as well as h38C2_K99Y DVD-Fab, which was cloned, expressed, and purified analogous to h38C2_K99C DVD-Fab. Using a molar ratio of 1:5, the 3 proteins were incubated with the 3 compounds in 9 separate reactions for 1 h at room temperature (RT) in PBS pH 8.5. Note the omission of a reducing reagent commonly used for bioconjugation to the thiol group of natural and engineered cysteine residues. Following bioconjugation, analysis by nonreducing SDS-PAGE and blue light visualization revealed a strong fluorescent band of ˜70 kDa for the bioconjugation of compounds 1 and 2 to h38C2_K99C DVD-Fab and parental h38C2 DVD-Fab but not h38C2_K99Y DVD-Fab (FIG. 1D). This result indicated that maleimide and monobromomaleimide can react with both uniquely reactive lysine and cysteine residues in the deep pocket of h38C2. Interestingly, bioconjugation of compound 3 was only observed for h38C2_K99C DVD-Fab, suggesting that dibromomaleimide selectively reacts with the engineered cysteine residue (FIG. 1D). Following this initial qualitative analysis, the efficiency of bioconjugating compounds 1, 2, and 3 to h38C2_K99C DVD-Fab was assessed by mass spectrometry. Compound 5, a fluorescein derivative with methylsulfone phenyloxadiazole (MS-PODA) functionality (FIG. 3), was included. MS-PODA::cysteine conjugates are more stable than maleimide::cysteine conjugates (thiosuccinimides) which readily hydrolyze through a retro-Michael reaction [18]. We showed recently that derivatives with MS-PODA functionality can also be rapidly, efficiently, and selectively conjugated to the reactive lysine residue of h38C2 [12]. The molecular weight of the unconjugated h38C2_K99C DVD-Fab was measured as 73,782 Da (expected 73,776.5 Da), and the conjugated DVD-Fabs h38C2_K99C_2, h38C2_K99C_3, and h38C2_K99C_5 gave major peaks at 74,545 Da (55%), 74,625 Da (90%), and 74,475 Da (55%), respectively, revealing a single conjugation event (FIG. 7). Notably, the difference in molecular weight between DVD-Fabs h38C2_K99C_2 (monobromomaleimide) and h38C2_K99C_3 (dibromomaleimide) was 80 Da which corresponds to one bromine atom (79.9 Da). Their molecular weight further suggested that both the monobromomaleimide::cysteine conjugation of 2 and the dibromomaleimide::cysteine conjugation of 3 proceeded by substituting one bromine to afford the thioether of the thio-monobromomaleimide (FIG. 1E). Although compound 1 revealed a similar bioconjugation efficiency to h38C2_K99C DVD-Fab as compounds 2 and 3 by nonreducing SDS-PAGE and blue light visualization (FIG. 1D), mass spectrometry revealed the unconjugated antibody as the major peak of this reaction (FIG. 7). Collectively, dibromomaleimide emerged as a superior electrophile for efficient site-specific bioconjugation to the buried cysteine residue of h38C2_K99C DVD-Fab. One-hour reaction time of dibromomaleimide bioconjugation to h38C2_K99C DVD-Fab is fast compared to other assembly strategies that sometimes require 24 hours or more reaction time.

### Example 4: Further Analysis of h38C2_K99C_3 DVD-Fab

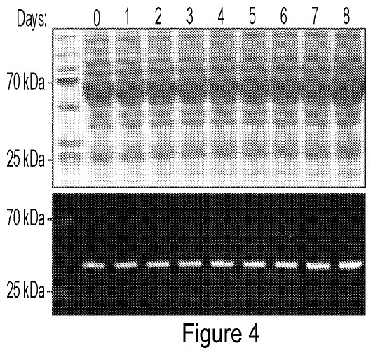

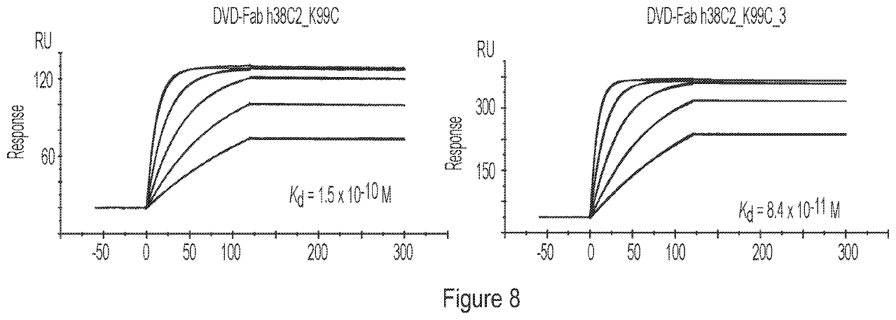

As the TAMRA derivative with dibromomaleimide functionality revealed the highest efficiency and selectivity for h38C2_K99C DVD-Fab compared to the parental h38C2 DVD-Fab, we sought to further analyze its utility for antibody-drug conjugates. First, the stability of h38C2_K99C_3 was assessed by incubation in human plasma at 37° C. for up to 10 days. As shown in FIG. 4, no significant loss of fluorescence was detected, suggesting high stability. As noted above, depending on the location of the cysteine, these conditions promote the retro-Michael hydrolysis of unsubstituted thiosuccinimides, with the freed maleimide reacting with the unpaired cysteine 34 of human serum albumin (HSA) which is present in high concentration. However, unlike in previous studies and in line with high stability, no fluorescent labeling of the dominant 66.5-kDa HSA band was detectable (FIG. 4). Next, the integrity of the HER2-binding outer Fv before and after h38C2_K99C_3 DVD-Fab assembly was tested by surface plasmon resonance (SPR). The affinity of the unconjugated h38C2_K99C DVD-Fab for immobilized recombinant HER2-Fc fusion protein was 150 nM (FIG. 8), consistent with a previous study that measured 160 nM for the parental h38C2 DVD-Fab [20]. The conjugated h38C2_K99C_3 DVD-Fab revealed an affinity of 84 nM (FIG. 8). Thus, neither mutation nor conjugation diminished HER2 binding.

### Example 5: Docking Simulations

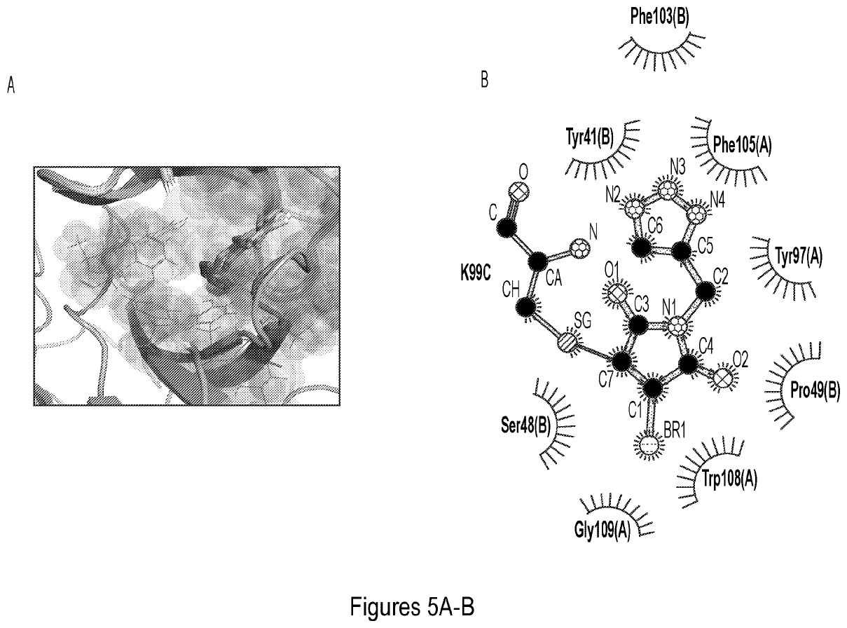

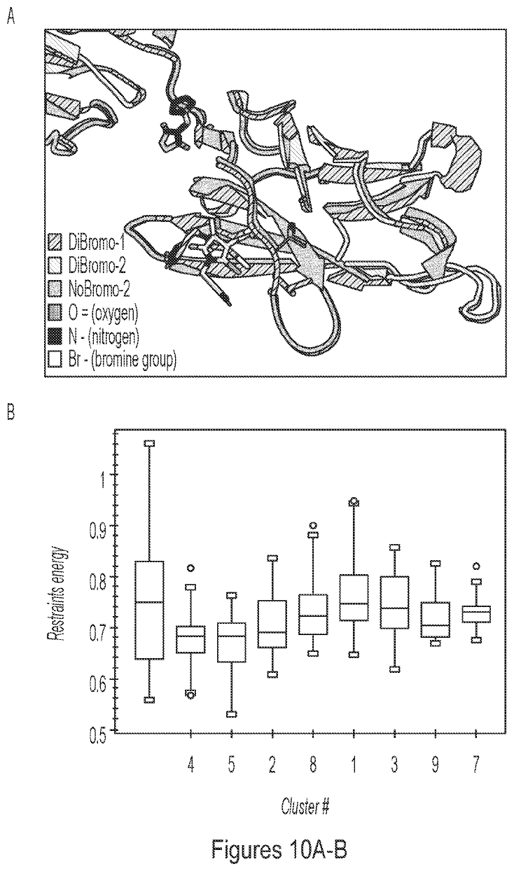

To understand the high efficiency and stability of dibromomaleimide conjugation to the buried cysteine, we used molecular protein-ligand docking to model maleimide, monobromomaleimide, and dibromomaleimide in the pocket of the crystallized h38C2_K99C Fab. Docking simulations of K99C and dibromomaleimide ligand are shown in FIGS. 5A-B. Mimicking compounds 1, 2, and 3 (FIG. 3), the neighboring triazole ring spaced by a methylene group from the maleimide functionality was included in the ligand. Initially, each of the compound-mimicking ligands, dubbed ‘NoBromo”, MonoBromo’, and ‘DiBromo’, were docked to the Fab pocket using noncovalent docking to isolate realistic poses that could allow for reactive chemistry. Subsequently, the covalent ligand docking platform HADDOCK was used to reveal the different clusters of binding modes. Clusters of different poses were created from the top 200 binders, only based on their relative RMSD, allowing for an energetically unbiased nature classification. All of the docking simulations were performed with minimal restraint energies, validating the in silico binding events as true events of preferential interactions, while the corresponding solvation process was used as an efficient way for dissociating any weak binders away from the active site. In case of ‘MonoBromo’, we observed three clusters out of nine to have dissociated in the presence of the solvent, despite having similar restraint energies imposed on all (FIG. 10B). Among the rest of the clusters, only one showed optimal binding mode, with adherence at the binding pocket, correct orientation as well as optimal bonding parameters. Interestingly, ‘DiBromo’ only segregated into two clusters, both binding at the expected pocket suggesting a higher inherent affinity of the compound 3-mimicking ligand. Also, the two clusters were easily identifiable as simple rotational isomers of each other, providing the rationale for such stabilization (FIG. 10A—DiBromo-1 docking simulation hatched with lines angled to left and dotted, DiBromo-2 docking simulation hatched with lines angled to right). In clear contrast, ‘NoBromo’ showed only a single cluster, away from the active site, completely dissociated from the thiol group of the cysteine (FIG. 10A—NoBromo-2 docking simulation hatched with dotted pattern). While it might be tempting to think that 3 is stabilized by the consecutive cysteines (C98 and C99) interacting with the two bromines, C98 is unable to participate in the stabilization of the ligand while simultaneously maintaining the intradomain disulfide bridge at the opposite face of the beta strand. While it might be possible for C99 to replace C98 in the intradomain disulfide bridge formation with C22, such an event will cost destabilization of the beta strand and possible loss of the ligand in the process. From this comparative analysis of the binding poses of the different maleimide-triazole complexes, we observed that ‘DiBromo’ preferentially bound to the cysteine, and its stabilization obtained by existence of rotational isomers leading to a better reaction efficiency.

### Example 6: Generation and Characterization of ADCs Based on h38C2_K99C IgG1

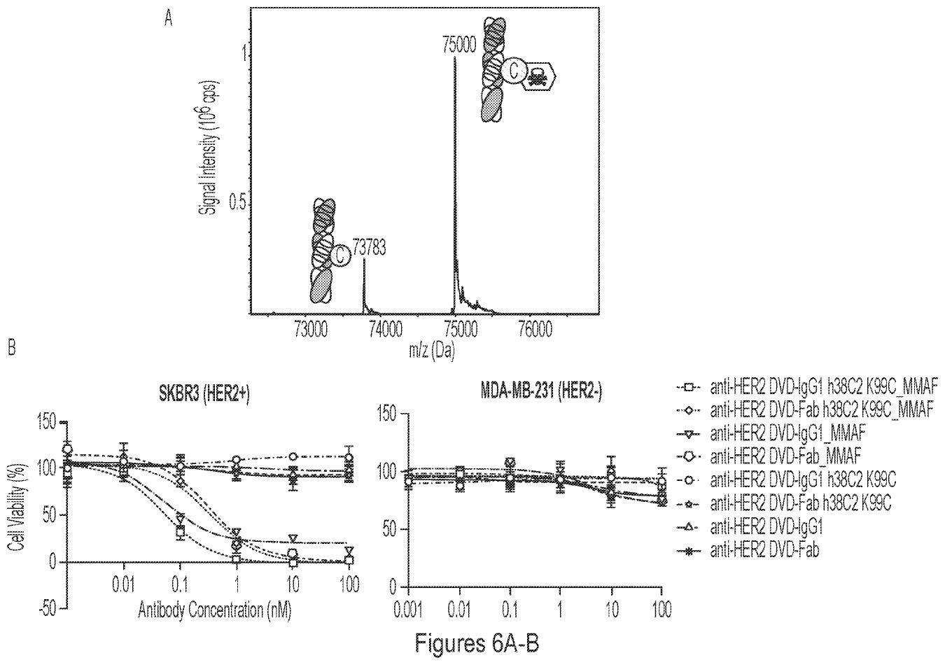

To investigate the suitability of the h38C2_K99C module for assembling ADCs, a monomethylauristatin F (MMAF) derivative with dibromomaleimide functionality was synthesized (compound 4; FIG. 3). Compound 4 was bioconjugated to the HER2-targeting h38C2_K99C DVD-Fab under the same conditions used for compound 3. The drug-to-antibody (DAR) ratio of the assembled h38C2_K99C_4 DVD-Fab was analyzed my mass spectrometry. Compared to the unconjugated h38C2_K99C DVD-Fab (73,783 Da), the conjugated antibody showed a major peak at 75,000 Da, revealing the site-selective conjugation of one drug (FIG. 6A). Thus, the DAR was calculated as 0.9. Next, the cytotoxicity of the HER2-targeting h38C2_K99C_4 DVD-Fab and DVD-IgG1 was compared to the corresponding ADC with the parental h38C2 module conjugated to β-lactam-MMAF [13]. A concentration range of the ADCs, along with their unconjugated antibody carriers, was incubated with HER2-positive SK-BR-3 and HER2-negative MDA-MB-231 breast cancer cell lines. Demonstrating equivalent picomolar potency toward the HER2-positive cells, the IC₅₀ values of the ADCs were indistinguishable at 0.052 and 0.054 nM (h38C2_K99C DVD-IgG1 and h38C2 DVD-IgG1), and 0.333 and 0.329 nM (h38C2_K99C DVD-Fab and h38C2 DVD-Fab). Neither ADC killed HER2-negative cells, and the antibody carriers alone did show no cytotoxicity (FIG. 6B). Similar results were observed for the corresponding CD79B-targeting ADCs in cytotoxicity assays with a lymphoma cell line. Collectively, the h38C2_K99C module affords an efficient and stable assembly route for site-specific ADCs and could be used in combination with the parental h38C2 or the h38C2_K99R module to build antibody conjugates with dual payloads [21, 22].

### Example 7: Experimental Procedures

### Cell Lines

Breast cancer cell lines SK-BR-3 and MDA-MB-231 were purchased from ATCC and cultured in DMEM medium supplemented with 10% (v/v) heat inactivated FBS and 1× penicillin-streptomycin (containing 100 U/mL penicillin and 100 mg/mL streptomycin; all from Thermo Fisher). Expi293F cells were cultured in Expi293 expression medium supplemented with 1× penicillin-streptomycin (all from Thermo Fisher).

Cloning, Expression, and Purification of h38C2_K99C Fab and DVD-Fab

Fab. Light chain (V_{κ}-C_{κ}; LC) and heavy chain fragment (V_{H}-C_{H}1; Fd) encoding sequences of h38C2 Fab with a Lys99Cys (K99C) and Lys99Tyr (K99Y) mutation in V_{H} and an N-terminal human CD5 signal peptide (MPMGSLQPLATLYLLGMLVASVLA; SEQ ID NO: 15) encoding sequence were separately cloned via NheI/XhoI (New England Biolabs) into mammalian expression vector pCEP4. Purified (Qiagen) plasmids encoding LC and Fd were co-transfected into Expi293F cells, which had been grown in 300 mL Expi293 Expression Medium to a density of 3×10⁶ cells/mL, using the ExpiFectamine 293 Transfection Kit (Thermo Fisher) following the manufacture's instruction. After continued culturing in 300 mL Expi293 Expression Medium at 37° C., 5% CO₂ for 5 days, the culture supernatant was collected and purified by affinity chromatography with a 1-mL HiTrap KappaSelect column in connection with an ÄKTA FPLC instrument (both from GE Healthcare). The yield of Fab was ˜15 mg/L as determined by the Pierce BCA Protein Assay Kit (Thermo Fisher). The Fab was further purified by size-exclusion chromatography using a Superdex 200 10/300 GL column (GE Healthcare) connected to the ÄKTA FPLC instrument. Fab peak fractions were concentrated by an Amicon Ultra 0.5-mL Centrifugal Filter (MilliporeSigma) and brought into 0.1 M sodium acetate (pH 5.5).

DVD-Fab and DVD-IgG1. The same LC and Fd expression cassettes as for the Fab extended by V_{κ} and V_{H} outer domain encoding sequences of trastuzumab were cloned to generate a HER2-targeting h38C2_K99C_DVD-Fab and h38C2_K99C DVD-IgG1. Following expression in the Expi293F system described above, the culture supernatant was collected and purified by affinity chromatography with a 1-mL Protein A HP column (GE Healthcare) in conjunction with the ÄKTA FPLC instrument. The yield of DVD-Fab was ˜18 mg/L as determined by the Pierce BCA Protein Assay Kit. To confirm its purity, reduced and nonreduced protein was subjected to SDS-PAGE using a 10-well NuPAGE 4-12% Bis-Tris Protein Gel followed by staining with PageBlue Protein Staining Solution (all from Thermo Fisher).

Crystallization and Structure Determination of h38C2_K99C Fab

Crystals were obtained by vapor diffusion at RT from a precipitant condition containing 20% (w/v) PEG 3350, 200 mM ammonium sulfate, and 100 mm Bis-Tris (pH 5.5). A diffraction data set with Bragg spacings to 2.4 Å was collected on a Dectris EIGER X 9M detector at the 21-ID-D beamline at the Advanced Photon Source (APS) synchrotron facility (Argonne National Laboratory). Molecular replacement solution was obtained using PDB ID 6U85 as a search model in PHASER. Crystallographic refinement was performed using a combination of PHENIX 1.2. Manual rebuilding, model adjustment, and real space refinements were done using the graphics program COOT. Model figures were created using PyMOL (Schrödinger). The coordinates and structure factors for the final model were deposited in the PDB under ID 7TUS.

### Docking Simulations

Creation of maleimide derivative ligands: 1N2 ligand from an analogous structure mimicking the modified cysteine ligand (PDB ID: 5CZD) was isolated to create the appropriate ligand. For docking simulations, the ligand was modified to contain two 5-membered aromatic rings: one linking the maleimide section to the reactive thiol group of the cysteine and the other containing three nitrogen atoms. These rings are expected to be planar to maintain aromaticity and were linked with a methylene group. The modifications allowed for the generation of a minimal double-ring scaffold that was modified according to the availability of bromine substitutions at the maleimide ring at C1 and C7 positions.

Initial docking via Rosetta: The ROSIE platform from the Gray laboratory at Johns Hopkins University was used to perform the non-constrained ligand docking at the h38C2_K99C Fab. Ligand conformations were expanded using a BCL library generation step to allow for minimum bias of the starting conformer, and subsequently, the randomization of the initial position was restricted to 0 Å. This allowed for monitoring a low resolution grid of 10 Å with a total cycle of 50 dockings.

Isolation of correct orientation of ligands: The docked structures were investigated for cross-docking orientations ignoring the thiol hydrogen atom to isolate 14 realistic poses. The structures were used as a template for Molecular Replacement (MR) using the PHASER module in PHENIX to naturally relax h38C2_K99C Fab. Followed by relaxation of the protein, the READYSET module in PHENIX was used to generate primary rigid-body restraints on the docked and post-MR models. The READYSET output was geometrically optimized to choose the best template to use for covalent docking.

Covalent Docking: The selected best candidate was used to initiate the HADDOCK covalent docking protocol, which allowed for a modified energy function, such that the thiol hydrogen of the cysteine is reduced in size to allow for minimal contribution of electrostatic and van der Waals clashes. The K99C reside was relabeled as CYC for the appropriate energy parameters and the residue accessibility threshold was set at 5.0. We also restricted 180° flips and fixed orientation during the rigid body minimization step and the electrostatic repulsion weight was minimized from 1.0 to 0.1. The restraints on the C—S bond were separately incorporated with 1.8 Å as the optimal bond length with ±0.1 Å as allowed deviation. Dihedral restraints of 0° were added to maintain the sulfur of the cysteine and the carbonyls of the maleimide on the same plane and in a realistic conformation.

### Catalytic Activity Assay

Catalytic activity was analyzed using methodol and carried out as described previously [23].

### Synthesis of Maleimide and β-Lactam Derivatives

The syntheses of compounds 1 (Maleimide-TAMRA), 2 (Monobromomaleimide-TAMRA), 3 (Dibromomaleimide-TAMRA)), and 4 (Dibromomaleimide-MMAF) and their validation by HPLC, MS, and NMR is provided in the Supplementary Information. The synthesis of compound 5 (MS-PODA-fluorescein) and β-lactam-hapten-MMAF was described previously.

### Antibody Conjugation

10 μM h38C2_K99C, h38C2_K99Y and parental DVD-Fab targeting HER2 antigen were incubated with 50 μM compound 1, 2, 3 in PBS (pH 8.5) for 1 h at RT. 7.5 μg of each conjugation mixture was loaded onto a 10-well NuPAGE 4-12% Bis-Tris Protein Gel. Fluorescent bands were visualized by blue light on an E-gel Imager (Thermo Fisher) and the gel was subsequently stained by PageBlue Protein Staining Solution. To generate ADCs in DVD-IgG1 format, 10 μM h38C2_K99C DVD-Fab and h38C2_K99C DVD-IgG1 were incubated with 50 and 100 μM of compound 4 in PBS (pH 8.5) for 1 h at RT. In parallel, 10 μM h38C2 DVD-IgG1 was incubated with 100 μM β-lactam-hapten-MMAF at RT for 4 h. Following incubation, PD MiniTrap G-25 (Cytiva) was used to remove free compounds and the ADCs were concentrated with Amicon Ultra 0.5-mL Centrifugal Filters above 1 mg/mL in PBS (pH 7.4).

### Mass Spectrometry

Following conjugation of 5 equivalents of compound 1, compound 2, compound 3, or compound 4 to h38C2_K99C DVD-Fab as described above, PD MiniTrap G-25 were used to remove free compound and the conjugated DVD-Fab was concentrated at 1 mg/mL in PBS by using Amicon Ultra 0.5-mL Centrifugal Filters. Followed by diluting into water, data were obtained by an Agilent Electrospray Ionization Time of Flight (ESI-TOF) mass spectrometer and deconvoluted masses were achieved by using Agilent BioConfirm Software.

### Human Plasma Stability Assay

To investigate a stability of antibody-fluorophore conjugates in human plasma, 1 mg/mL of h38C2_K99C and parental DVD-Fab conjugated to compound 3 was mixed with an equal volume of human plasma (Sigma-Aldrich) and incubated at 37° C. After 0, 6, 12, 24, 48, 96, 122, 196, and 240 h, 2-μL aliquots were frozen and stored at −80° C. After aliquots from all time points had been collected, samples were mixed with a reducing agent and subjected to 10-well NuPAGE 4-12% Bis-Tris Protein Gel. Fluorescence signals were visualized by blue light on an E-gel Imager (Thermo Fisher) and the gel was subsequently stained by PageBlue Protein Staining Solution. The experiment was carried out three times independently.

### Surface Plasmon Resonance

The kinetic and thermodynamic parameters of the binding of unconjugated and conjugated DVD-Fab to HER2 were measured by an operation of a Biacore X100 instrument (GE Healthcare). A mouse anti-human IgG C_{H}2 mAb was immobilized on a CM5 sensor chip (GE Healthcare) and human HER2-Fc fusion protein (R&D Systems) was captured on the chip at less than 300 RU density. All binding assays used 1×HBS-EP+ running buffer (10 mM HEPES, 150 mM NaCl, 3 mM EDTA (pH 7.4), and 0.05% (v/v) Surfactant P20) and a flow rate of 30 μL/min. All DVD-Fab were injected at five different concentrations and the sensor chip was regenerated with 3 M MgCl₂ from the Human Antibody Capture Kit (GE Healthcare) in each cycle. Calculation of association (kₒₙ) and dissociation (k_{off}) rate constants was based on a 1:1 Langmuir binding model, and the equilibrium dissociation constant (K_{D}) was calculated from k_{off}/kₒₙ.

### Cytotoxicity Assay

SK-BR-3 and MDA-MB-231 cells were plated in 96-well tissue culture plates at 5×10³/well and 3×10³ cells/well. Ten-fold serially diluted ADCs and their corresponding unconjugated DVD-IgG1 (0.001-100 nM) were added to the cells and the plates were incubated at 37° C. in an atmosphere of 5% CO₂ for 72 h. Subsequently, cell viability was measured using CellTiter 96 Aqueous One Solution (Promega) following the manufacturer's instructions and plotted as a percentage of untreated cells. IC₅₀ values were calculated by GraphPad Prism software.

### Example 8: Additional Technical Information for the Exemplified Embodiments

### Synthesis of Maleimide Derivatives

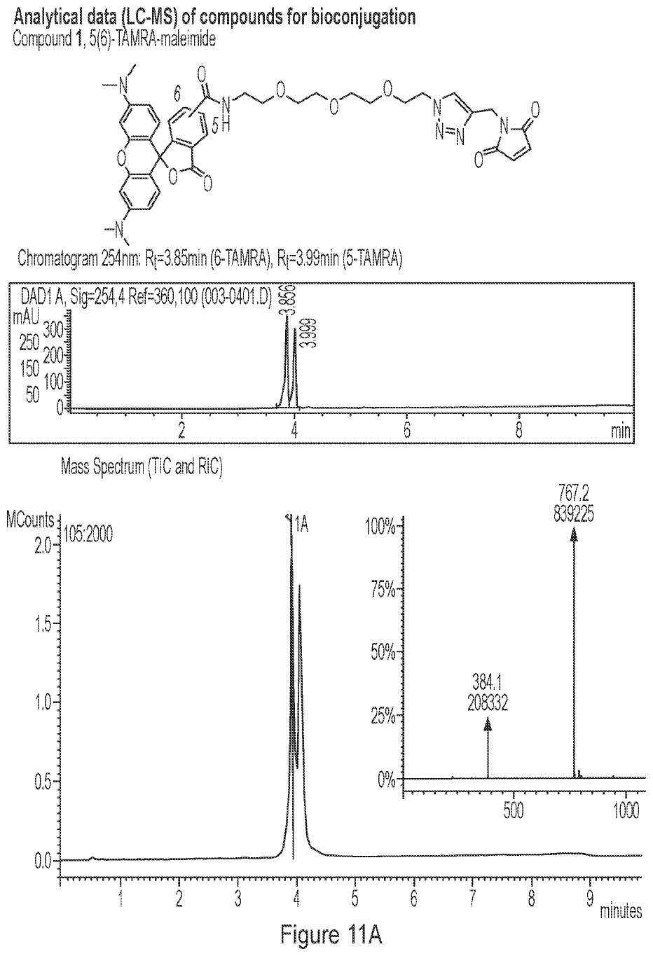

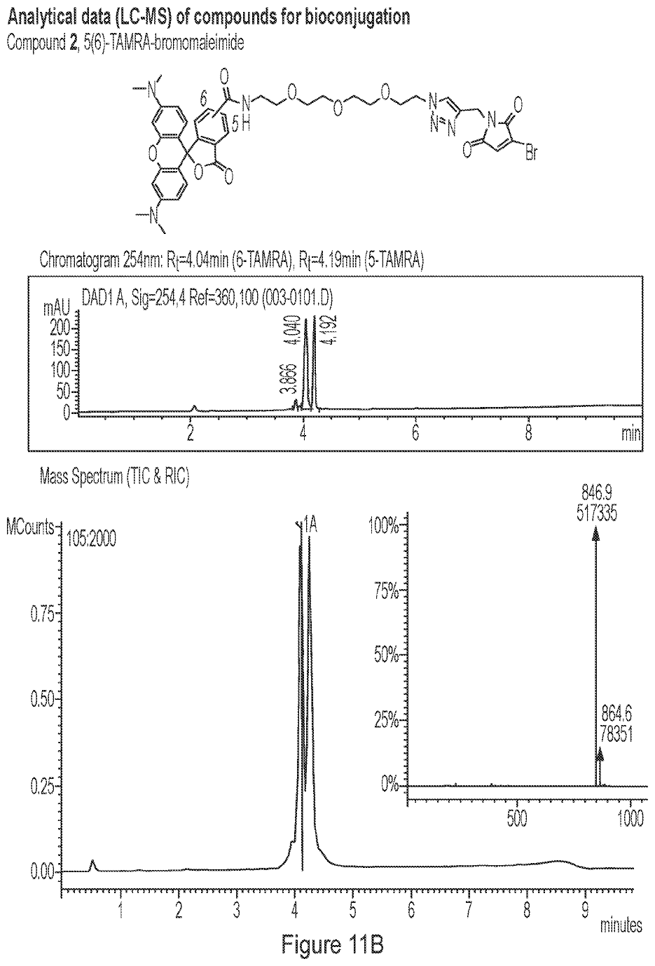

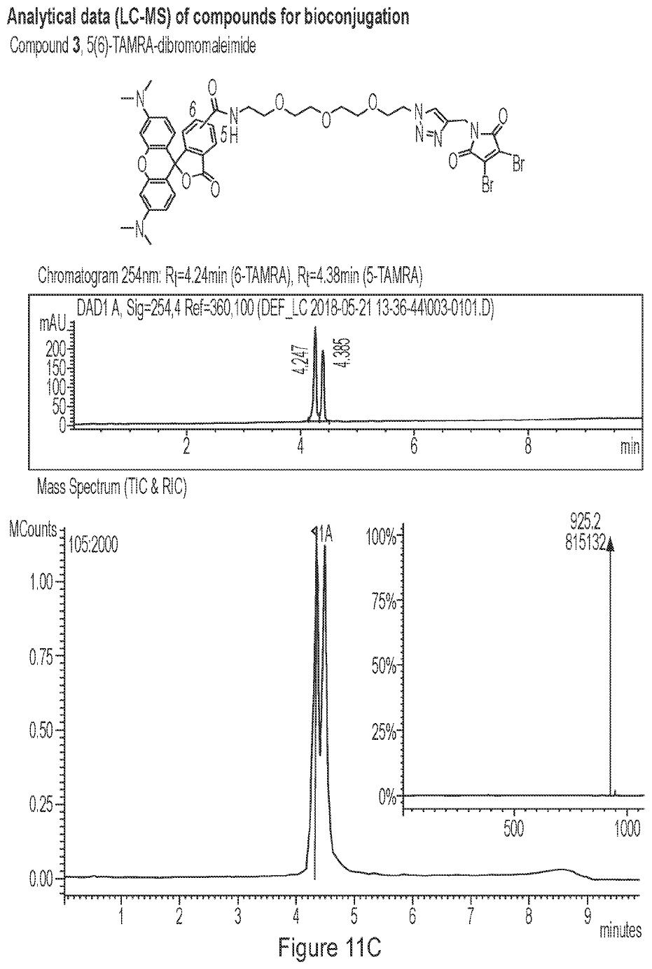

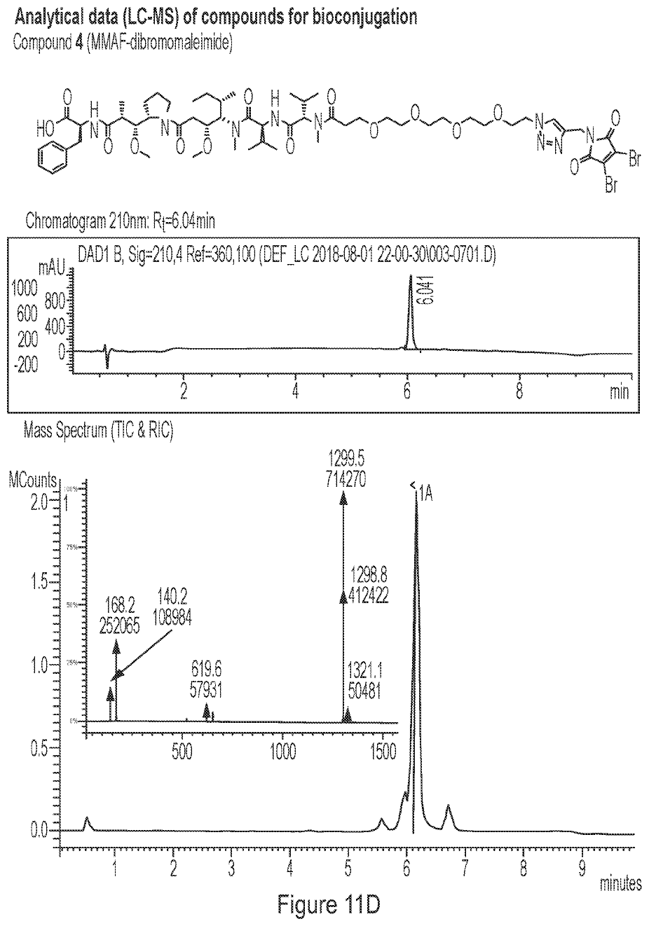

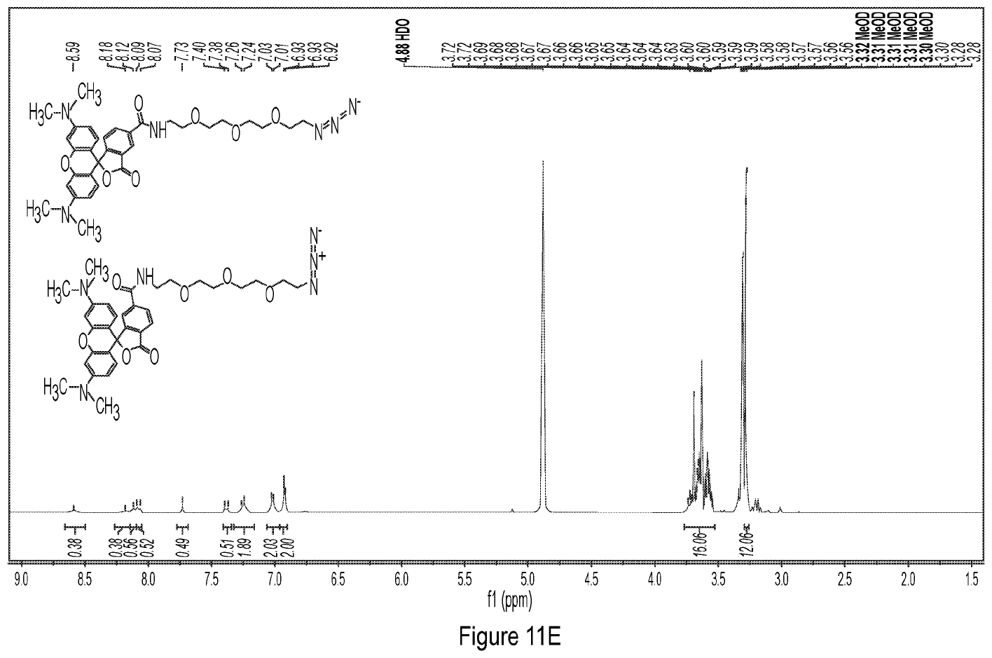

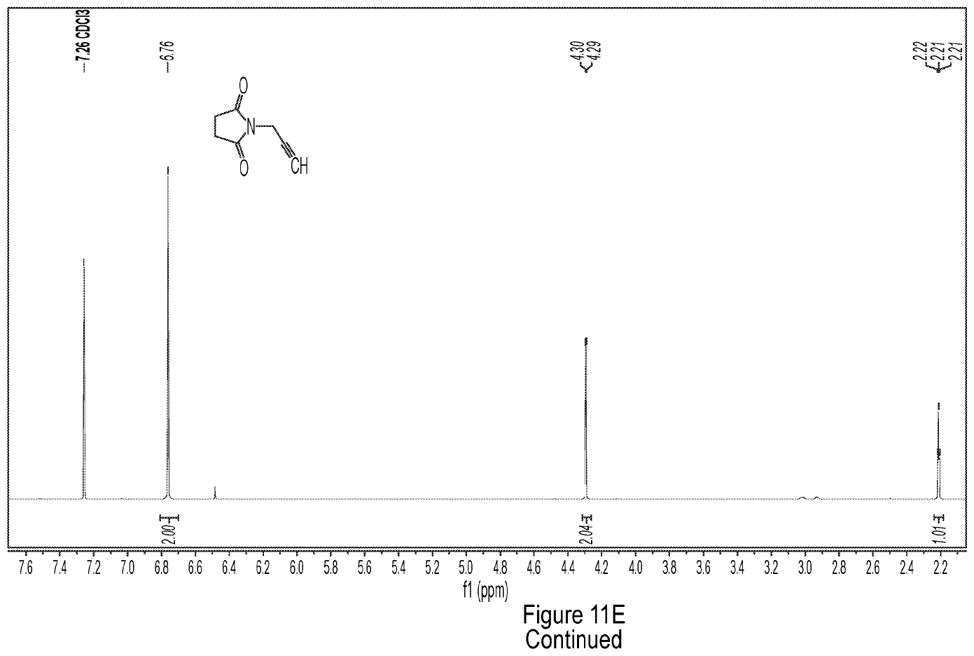

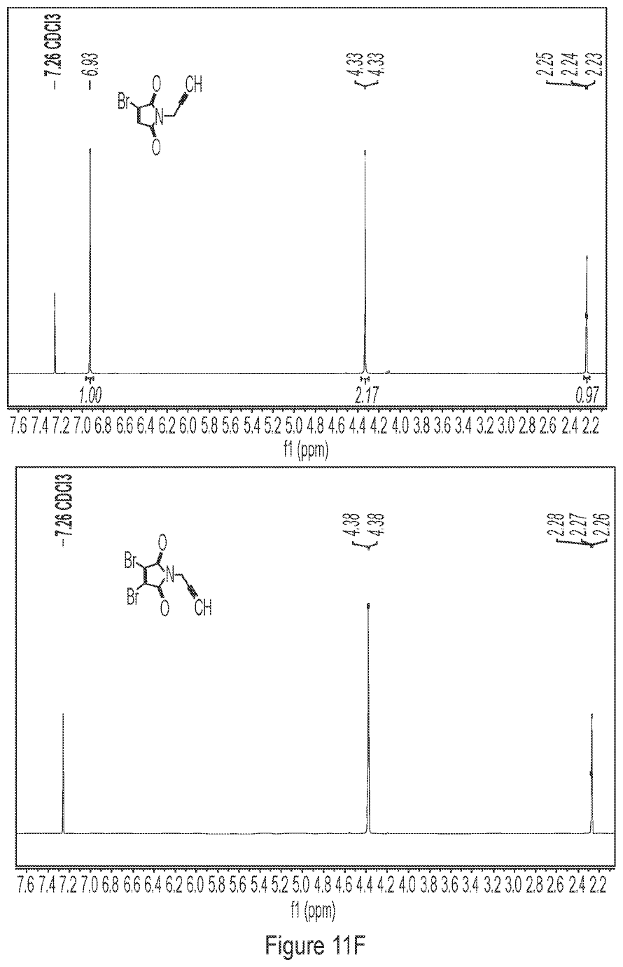

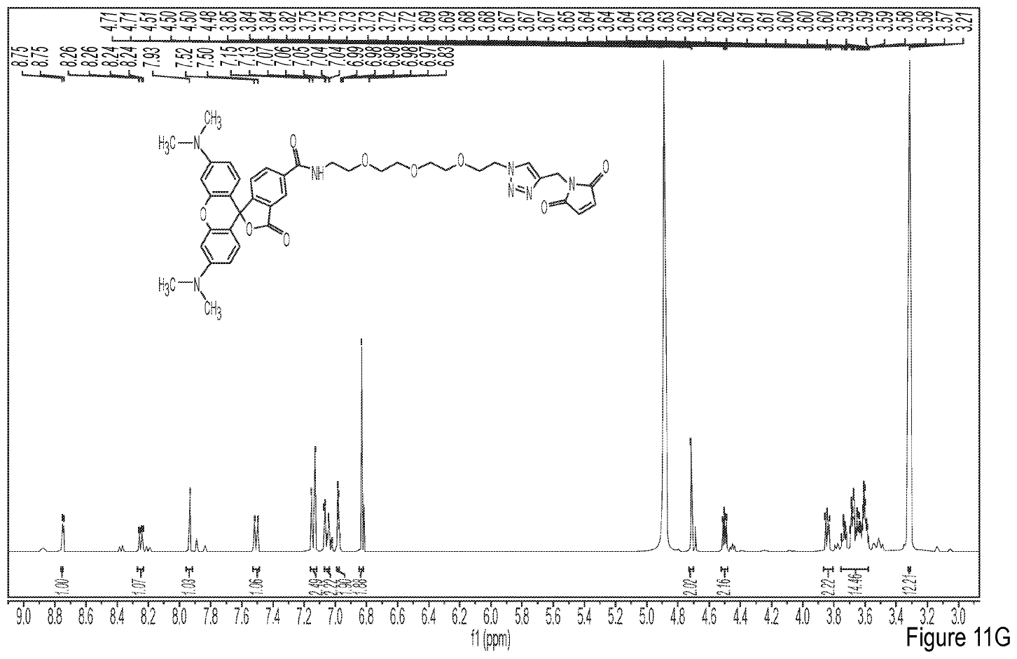

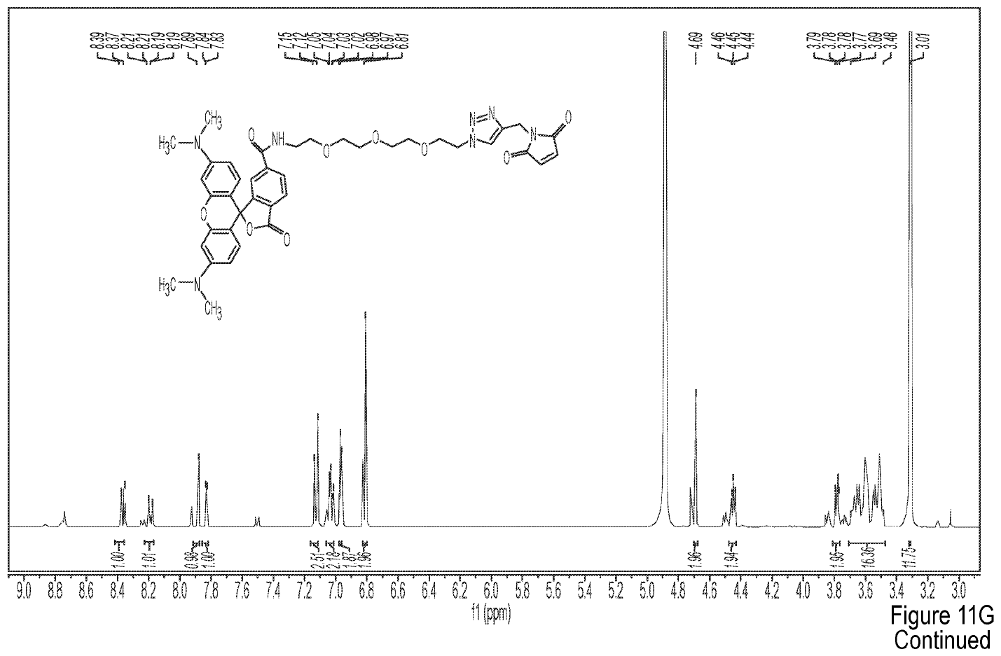

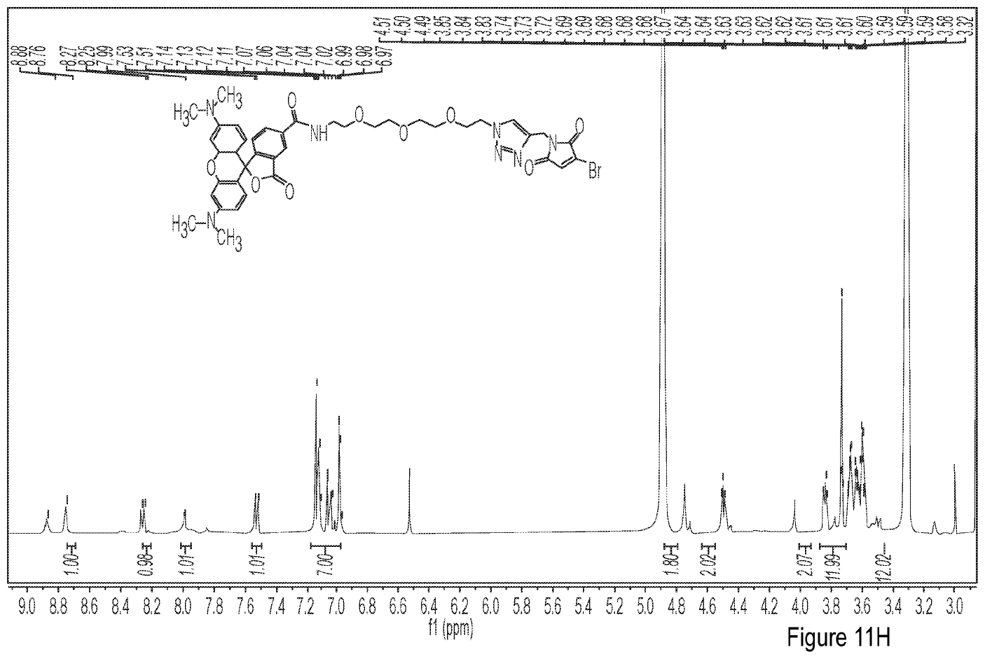

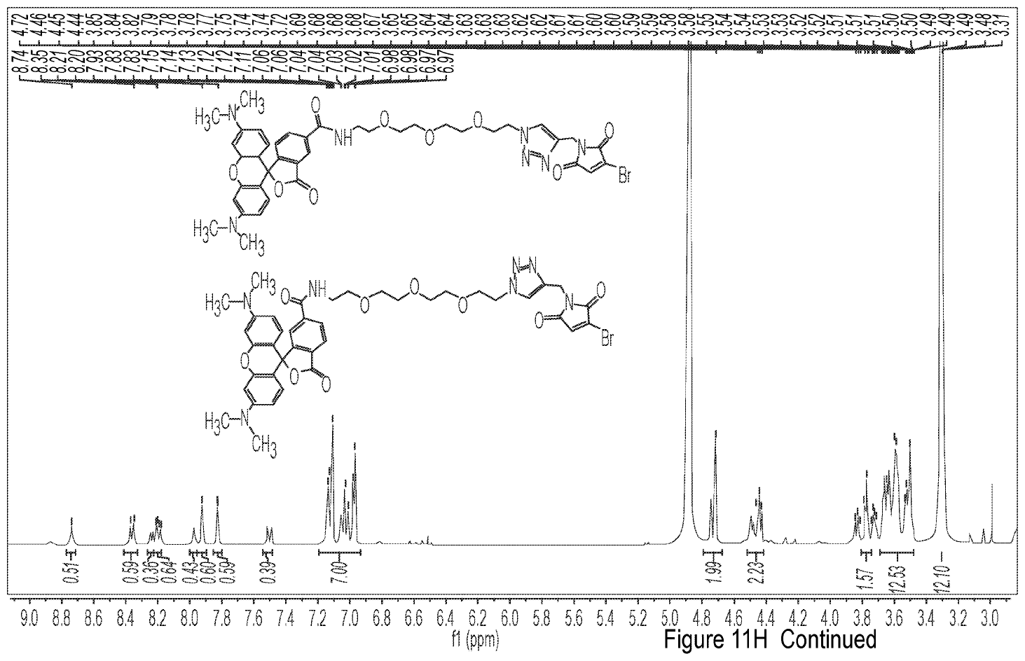

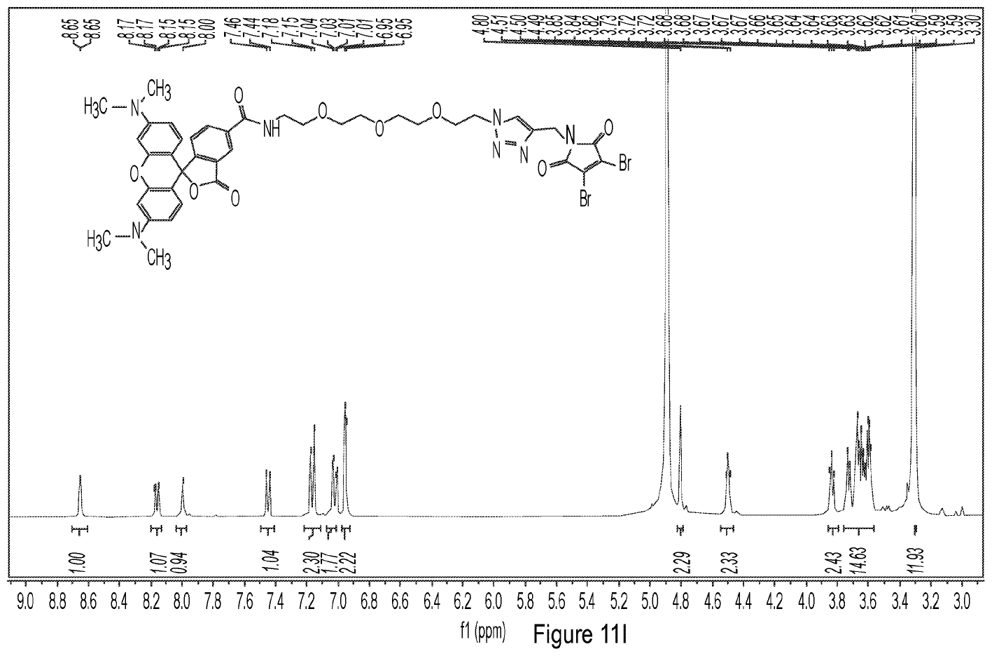

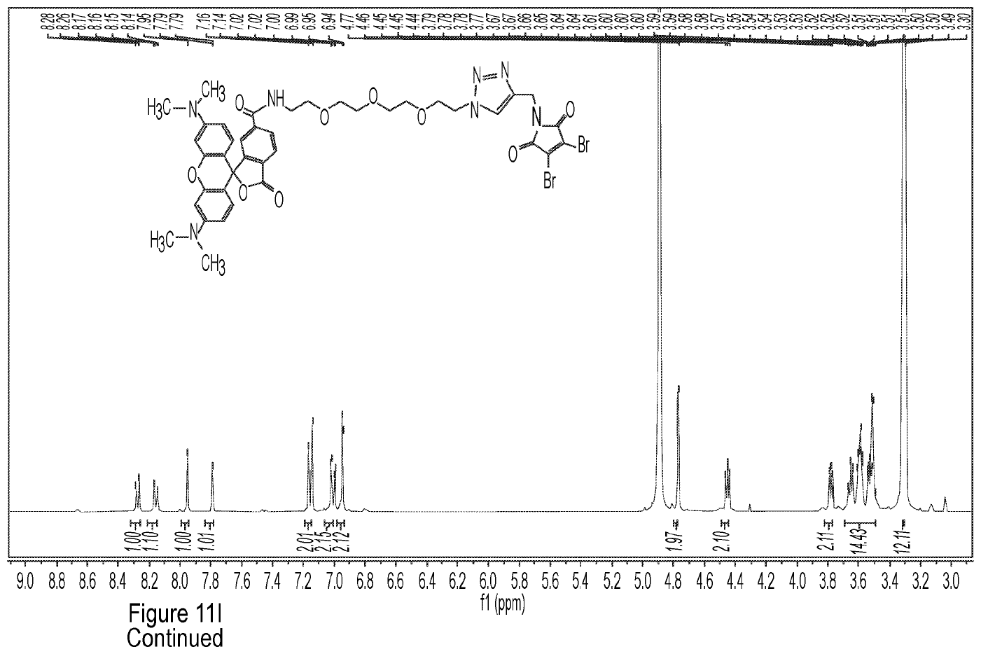

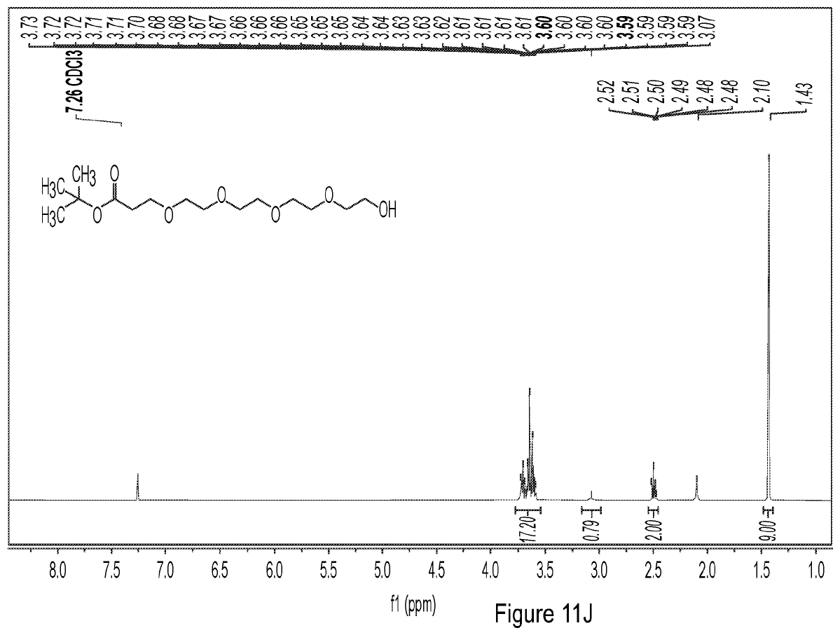

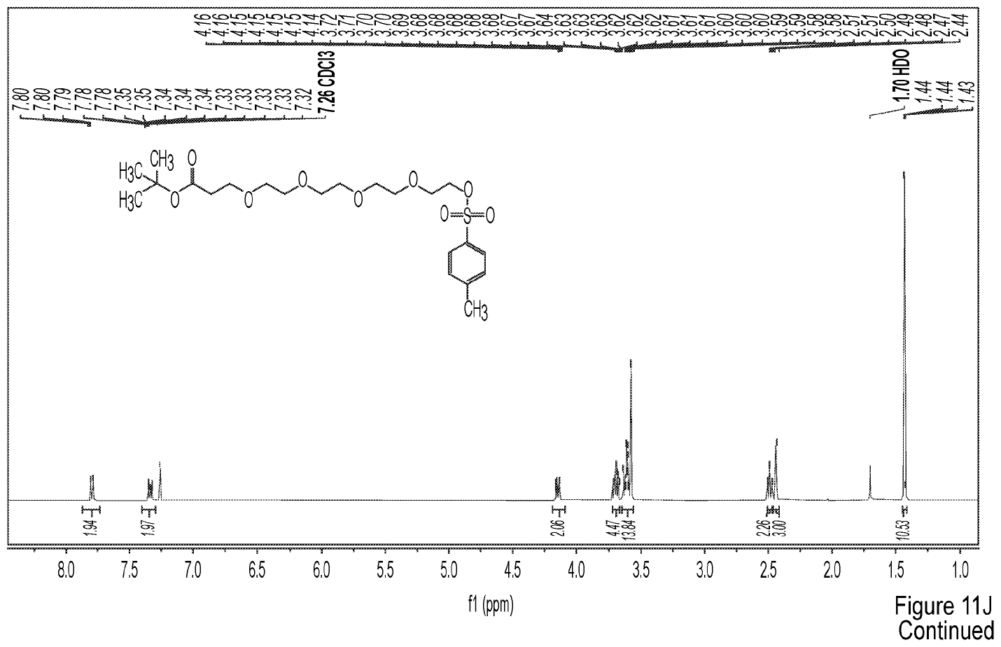

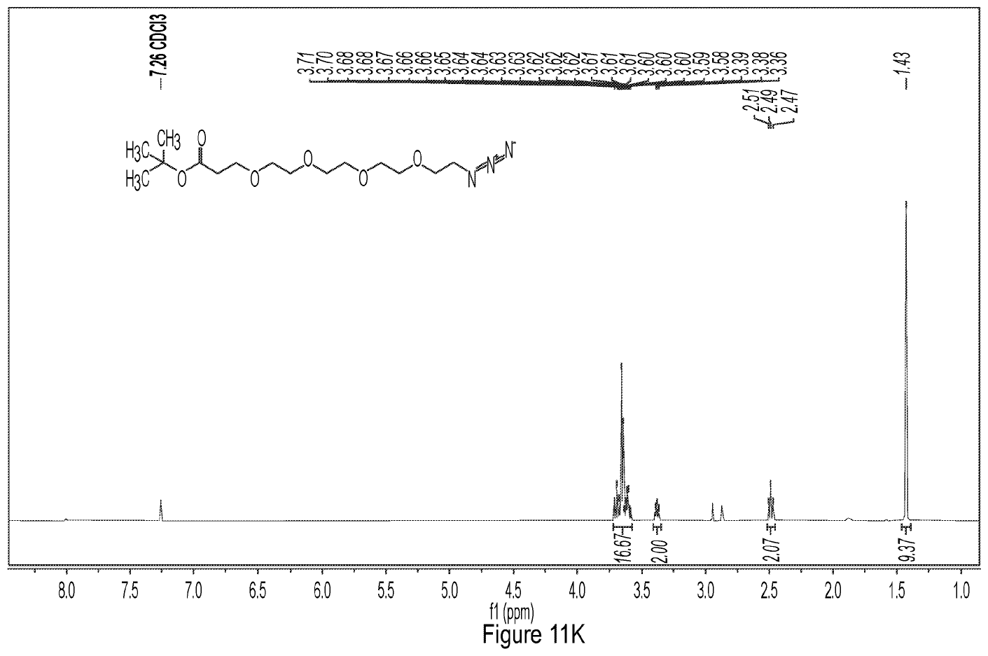

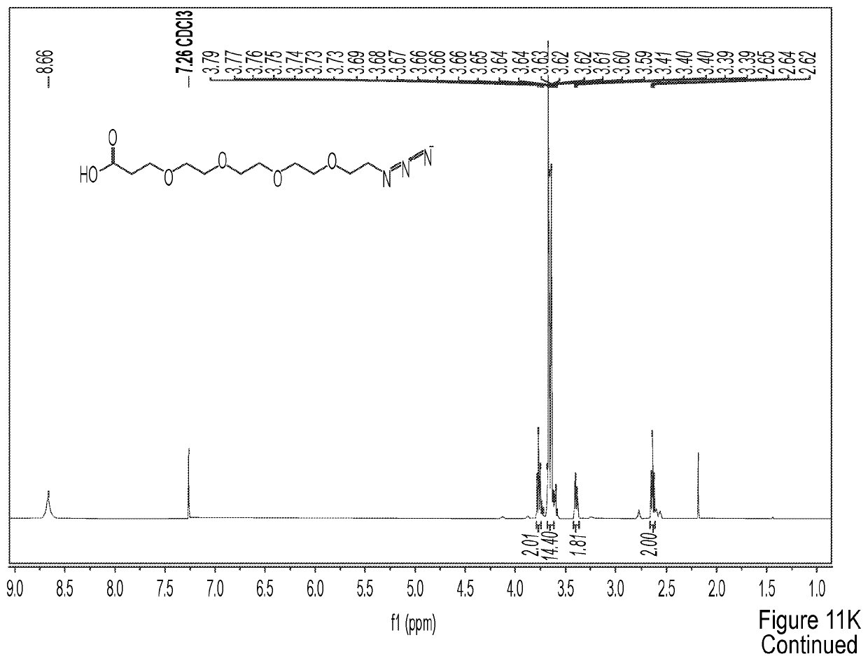

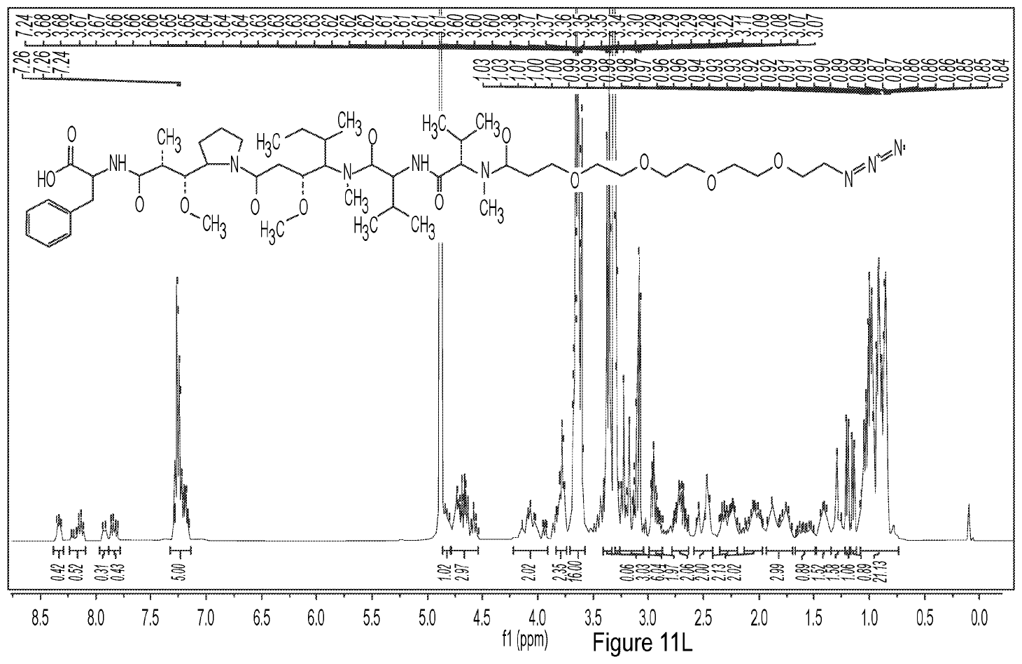

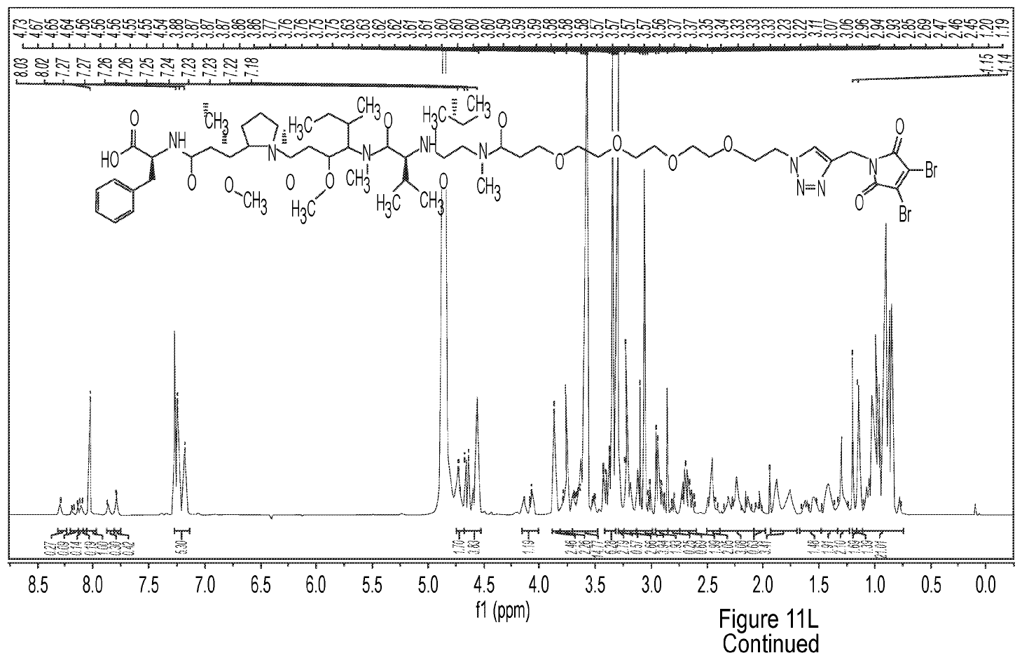

General Information. All non-aqueous reactions were performed in oven-dried or flame-dried glassware under argon atmosphere. Unless otherwise mentioned, all reagents were purchased from commercial suppliers and used without further purification. Dichloromethane, tetrahydrofuran and N,N-dimethylformamide were purified by passing through a solvent column of desiccant (Activated Alumina). Methanol was purchased as a reagent grade solvent. Diisopropylethylamine was distilled from calcium hydride under argon atmosphere. All reactions were monitored by either thin-layer chromatography or analytical liquid chromatography (LC)-mass spectrometry (MS). Thin layer chromatography was performed on Merck TLC silica gel 60 F254 glass plates pre-coated to 0.25-mm thickness. Visualization was done by UV light (254 nm), KMnO₄ stain, phosphomolybdic acid (PMA) stain, triphenylphosphine solution, and/or ninhydrin stain. Purification by preparative thin-layer chromatography was performed on Analtech UNIPLATE silica gel GF UV 254 20×20 cm, 2000-μm thickness. Purification by silica gel column chromatography was performed on SiliFlash F60 (40-63 μm, 230-400 Mesh). Preparative HPLC purifications were performed on a Shimadzu LC-8A preparative liquid chromatography system with mobile phase A as H₂O and mobile phase B as CH₃CN. 1H-NMR spectra were recorded on a Bruker 400 MHz or 700 MHz spectrometer in appropriate deuterated solvents. Chemical shifts were reported in parts per million (ppm) on the δ scale from residue solvent peaks. NMR descriptions: s, singlet; d, doublet; t, triplet; q, quartet; m, multiplet; br, broad). Coupling constants, J, are reported in Hertz (Hz). High-resolution mass spectra were obtained by electrospray ionization (ESI)-MS at the University of Illinois Urbana-Champaign Mass Spectrometry Laboratory. The purity of all materials used in biochemical and biological experiments were determined by analytical LC-MS to be >95%. FIGS. 11A, 11B, 11C, 11D, 11E, 11E continued, 11F, 11G, 11G, continued, 11H, 11H continued, 11I, 11I continued, 11J, 11J continued, 11K, 11K continued, 11L, and 11L continued present analytical data (LC-MS) of compounds for bioconjugation.

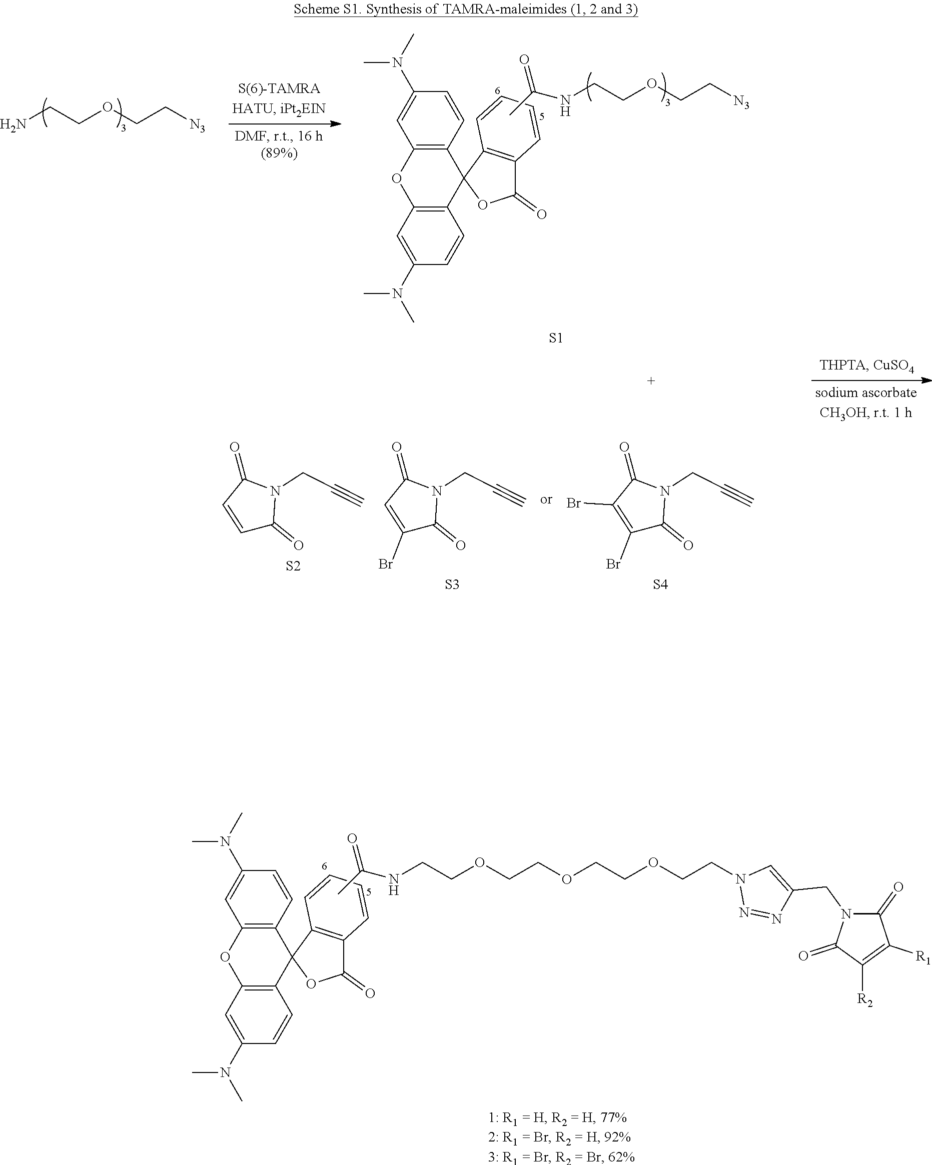

Synthesis of S1. 5(6)-TAMRA (20.0 mg, 0.046 mmol) and HATU (17.7 mg, 0.046 mmol) were dissolved in 250 μL. The mixture solution was added with DIPEA (40 μL, 0.229 mmol) and stirred at room temperature (r.t.) for 10 min. Then a solution of 2-(2-(2-(2-azidoethoxy)ethoxy)ethoxy)ethan-1-amine (11.15 mg, 0.051 mmol) in DMF (150 μL) was added dropwise to the activated TAMRA solution. The reaction was stirred at r.t. for 16 h. The crude reaction was purified by preparative TLC. The product bands were scraped off and stripped with 10% CH₃OH/CH₂Cl₂+0.05% TFA. Silica gel was filtered off and the filtrate was concentrated down to dryness. The viscous dark purple product was then redissolved in 0% iPrOH/CH₂Cl₂ and washed with water. (The pH of the aqueous layer was adjusted to 6-7 with saturated NaHCO₃). The aqueous layer was extracted with 10% iPrOH/CH₂Cl₂ (×5). The organic layer was combined, washed with brine, dried over anhydrous Na₂SO₄, and concentrated to dryness. The 5 and 6 isomer products (Scheme 1) were obtained as dark purple film coating the vial wall (26.2 mg, 89%). 1H-NMR of the mixture of 5 and 6 isomers (400 MHZ, Methanol-d4): δ 8.59 (s, br, 0.38H), 8.18 (s, br, 0.38H), 8.12 (s, 0.56H), 8.08 (d, J=7.8 Hz, 0.52H), 7.73 (s, 0.49H), 7.39 (d, J=7.8 Hz, 0.51H), 7.25 (d, J=8.8 Hz, 1.89H), 7.02 (d, J=8.6 Hz, 2.03H), 6.93 (t, J=2.00 Hz, 2H), 3.76-3.53 (m, 16.06H), 3.28 (s, 12.06H, partial overlap with solvent peak). HRMS calcd for C₃₃H₃₉N₆O₇ [M+H]+ 631.2896, found 631.2894.

Synthesis of S2. S2 was synthesized according to the procedure reported by [24]. 1H-NMR (400 MHZ, Chloroform-d) δ 6.76 (s, 2H), 4.29 (d, J=2.5 Hz, 2H), 2.21 (t, J=2.5 Hz, 1H).

Synthesis of S3. S3 was synthesized according to the procedure reported by [25]. 1H-NMR (400 MHZ, Chloroform-d) § 6.93 (s, 1H), 4.33 (d, J=2.5 Hz, 2H), 2.24 (t, J=2.5 Hz, 1H).

Synthesis of S4. S4 was synthesized according to the procedure reported [26]. 1H-NMR (400 MHZ, Chloroform-d) δ 4.38 (d, J=2.5 Hz, 2H), 2.27 (t, J=2.5 Hz, 1H).

Synthesis of 1. S1 (5.25 mg, 8.32 μmol) was mixed with S2 (2.25 mg, 0.017 mmol) in methanol (373 μL). Then aqueous solutions of 50 mM Tris(3-hydroxypropyltriazolylmethyl)amine (THPTA; 41.6 μL, 2.081 μmol), 50 mM copper (II) sulfate (41.6 μL, 2.081 μmol), and 100 mM sodium ascorbate (41.6 μL, 4.16 μmol) were added. Reaction completion was checked after 1 h by analytical LC-MS followed by purification with preparative HPLC. The fractions containing product were combined and evaporated to remove the organic solvent. The remaining aqueous solution was lyophilized to give the desired products as dark purple powder (4.9 mg, 77%, 5- and 6-isomer mixture). ¹H-NMR (400 MHZ, Methanol-d4) 5-isomer: δ 8.75 (d, J=1.8 Hz, 1H), 8.25 (dd, J=7.9, 1.9 Hz, 1H), 7.93 (s, 1H), 7.51 (d, J=7.9 Hz, 1H), 7.14 (d, J=9.5 Hz, 2H), 7.05 (dd, J=9.5, 2.4 Hz, 2H), 6.98 (d, J=2.4 Hz, 2H), 6.83 (s, 2H), 4.71 (s, 2H), 4.50 (dd, J=5.5, 4.6 Hz, 2H), 3.84 (dd, J=5.6, 4.6 Hz, 2H), 3.75-3.56 (m, 12H), 3.31 (s, 12H, overlap with solvent peak). 6-isomer: δ 8.38 (d, J=8.2 Hz, 1H), 8.20 (dd, J=8.2, 1.8 Hz, 1H), 7.89 (s, 1H), 7.84 (d, J=1.8 Hz, 1H), 7.13 (d, J=9.5 Hz, 2H), 7.04 (dd, J=9.5, 2.4 Hz, 2H), 6.97 (d, J=2.5 Hz, 2H), 6.81 (s, 2H), 4.69 (s, 2H), 4.45 (t, J=4.9 Hz, 2H), 3.78 (dd, J=5.6, 4.5 Hz, 2H), 3.59 (m, 12H), 3.31 (s, 12H, overlap with solvent peak). HRMS calcd for C₄₀H₄₄N₇O₉ [M+H]+ 766.3201 found 766.3167.

Synthesis of 2. S1 (5.25 mg, 8.32 μmol) was mixed with S3 (3.56 mg, 0.017 mmol) in methanol (373 μL). Then aqueous solutions of 50 mM THPTA (41.6 μL, 2.081 μmol), 50 mM copper(II) sulfate (41.6 μL, 2.081 μmol), and 100 mM sodium ascorbate (41.6 μL, 4.16 μmol) were added. Reaction completion was checked after 1 h by analytical LC-MS followed by purification with preparative HPLC. The fractions containing product were combined and evaporated to remove the organic solvent. The remaining aqueous solution was lyophilized to give the desired products as dark purple powder (6.5 mg, 92%, 5- and 6-isomer mixture). ¹H-NMR (400 MHZ, Methanol-d4) 5-isomer: δ 8.76 (s, 1H), 8.26 (d, J=7.9 Hz, 1H), 7.99 (s, 1H), 7.52 (d, J=7.9 Hz, 1H), 7.16-6.97 (m, 7H), 4.75 (s, 2H), 4.50 (t, J=4.9 Hz, 2H), 3.84 (t, J=5.0 Hz, 2H), 3.75-3.57 (m, 12H), 3.32 (s, 12H, overlap with solvent peak). 6-isomer: δ 8.36 (d, J=8.2 Hz, 1H), 8.20 (dd, J=8.1, 1.8 Hz, 1H), 7.93 (s, 1H), 7.83 (d, J=1.8 Hz, 1H), 7.19-6.93 (m, 7H), 4.72 (s, 2H), 4.45 (t, J=5.1 Hz, 2H), 3.78 (dd, J=5.5, 4.5 Hz, 2H), 3.70-3.48 (m, 12H), 3.31 (s, 12H, overlap with solvent peak). HRMS calcd for C₄₀H₄₃N₇O₉Br [M+H]+ 844.2306 found 844.2296.

Synthesis of 3. S1 (5.25 mg, 8.32 μmol) was mixed with S4 (4.88 mg, 0.017 mmol) in methanol (373 μL). Then aqueous solutions of 50 mM THPTA (41.6 μL, 2.081 μmol), 50 mM copper(II) sulfate (41.6 μL, 2.081 μmol), and 100 mM sodium ascorbate (41.6 μL, 4.16 μmol) were added. Reaction completion was checked after 1 h by analytical LC-MS followed by purification with preparative HPLC. The fractions containing product were combined and evaporated to remove the organic solvent. The remaining aqueous solution was lyophilized to give the desired products as dark purple powder (4.8 mg, 62%, 5- and 6-isomer mixture). ¹H-NMR (400 MHZ, Methanol-d4) 5-isomer δ 8.65 (d, J=1.9 Hz, 1H), 8.16 (dd, J=8.0, 1.6 Hz, 1H), 8.00 (s, 1H), 7.45 (d, J=7.9 Hz, 1H), 7.16 (d, J=9.5 Hz, 2H), 7.02 (dd, J=9.4, 2.5 Hz, 2H), 6.95 (d, J=2.5 Hz, 2H), 4.80 (s, 2H), 4.50 (t, J=5.0 Hz, 2H), 3.84 (t, J=5.0 Hz, 2H), 3.75-3.58 (m, 12H), 3.30 (s, 12H, overlap with solvent peak). 6-isomer δ 8.27 (d, J=8.2 Hz, 1H), 8.15 (dd, J=8.1, 1.8 Hz, 1H), 7.95 (s, 1H), 7.79 (d, J=1.8 Hz, 1H), 7.15 (d, J=9.5 Hz, 2H), 7.01 (dd, J=9.5, 2.5 Hz, 2H), 6.94 (d, J=2.4 Hz, 2H), 4.77 (s, 2H), 4.45 (dd, J=5.5, 4.4 Hz, 2H), 3.78 (d, J=5.4, 4.8 Hz 2H), 3.68-3.48 (m, 12H), 3.30 (s, 12H, overlap with solvent peak). HRMS calcd for C₄₀H₄₂N₇O₉Br₂ [M+H]+ 922.1411 found 922.1434.

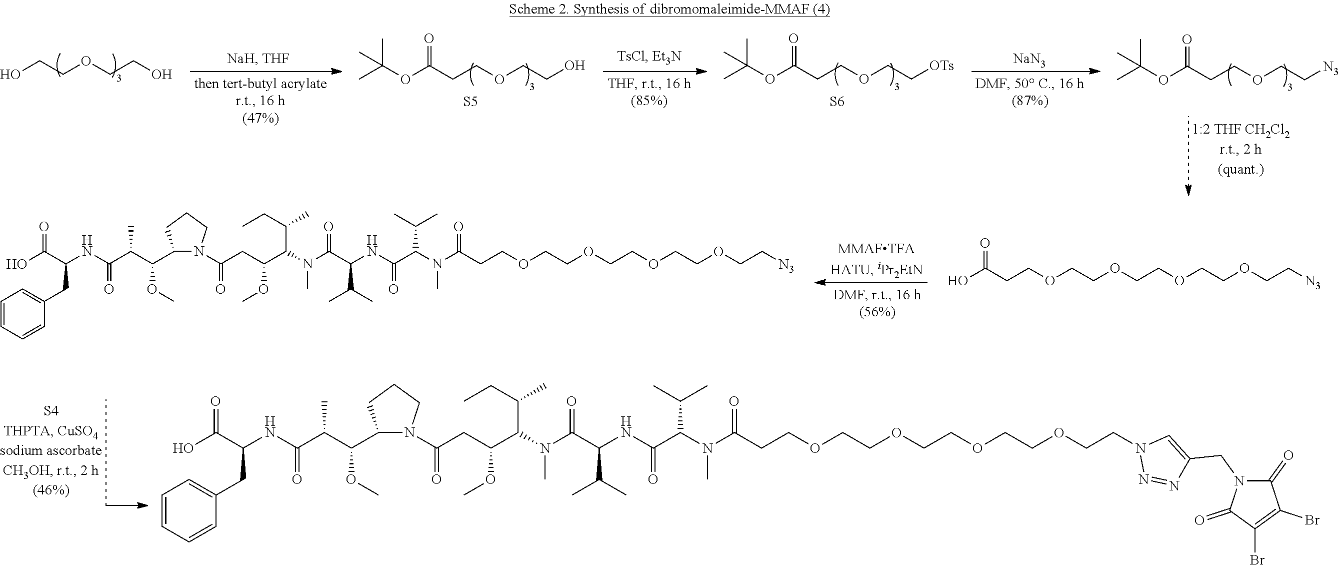

Synthesis of S5. Tetraethylene glycol (5 g, 25.7 mmol) in THF (10 mL) was added with sodium hydride (10.3 mg, 0.258 mmol), followed by tert-butyl acrylate (1.100 g, 8.58 mmol). The reaction was stirred at r.t. for 16 h. The solvent was removed under reduced pressure and the residue was purified with silica gel column chromatography. The product was obtained as clear colorless liquid (1.29 g, 47%). 1H-NMR (400 MHZ, Chloroform-d) δ 3.76-3.58 (m, 17H), 3.07 (s, br, 1H), 2.50 (t, J=6.6 Hz, 2H), 1.43 (s, 9H). HRMS calcd for C₁₅H₃₀O₇Na [M+Na]+ 345.1889 found 345.1879.

Synthesis of S6. S5 (800 mg, 2.481 mmol) was dissolved in THF (3 mL) and treated with triethylamine (1.038 mL, 7.44 mmol). The mixture was then added with a solution of tosyl chloride (568 mg, 2.98 mmol) in THF (2 mL) and a solution of 4-dimethylaminopyridine (DMAP; 30.3 mg, 0.248 mmol) in THF (300 μL). The reaction was stirred at r.t. for 2 h, then extracted with EtOAc, washed with water, brine, dried over anhydrous Na₂SO4, filtered, and concentrated to dryness. The product was purified with silica gel chromatography to obtain the desired product as clear colorless viscous liquid (1.00 g, 85%). ¹H-NMR (400 MHZ, Chloroform-d) δ 7.79 (d, J=8.3 Hz, 2H), 7.34 (d, J=7.9 Hz, 2H), 4.19-4.11 (m, 2H), 3.73-3.66 (m, 4H), 3.65-3.57 (m, 14H), 2.49 (t, J=6.6 Hz, 2H), 2.44 (s, 3H), 1.44 (s, 10H). HRMS calcd for C₂₂H₃₆O₉NaS [M+Na]+ 499.1978 found 499.1971.

Synthesis of S7. S6 (850 mg, 1.784 mmol) was dissolved in DMF (3.5 mL) and added with sodium azide (290 mg, 4.46 mmol). The suspension was stirred in a 50° C. oil bath for 16 h and then dried under vacuum. The pale-yellow residue was suspended in Et₂O. The pale-yellow precipitate was filtered off with a syringe packed with a plug of cotton wools. The clear yellow solution was collected and dried under reduced pressure. The residue was purified with silica gel column chromatography (50% EtOAc/hexanes to 100% EtOAc) to obtain the product as clear pale-yellow liquid (537.1 mg, 87%). ¹H-NMR (400 MHZ, Chloroform-d) δ 3.72-3.58 (m, 16H), 3.38 (t, J=5.1 Hz, 2H), 2.49 (t, J=6.6 Hz, 2H), 1.43 (s, 9H). HRMS calcd for C₁₅H₂₉N₃O₆Na [M+Na]+ 370.1954 found 370.1947.

Synthesis of S8. S7 (200 mg, 0.576 mmol) was dissolved in CH₂Cl₂ (1.8 mL) and then added with trifluoroacetic acid (887 μL, 11.51 mmol). The reaction was stirred at r.t. for 2 h and then co-evaporated with toluene (×3) and dried under high vacuum. The product was used without further purification. ¹H-NMR (400 MHZ, Chloroform-d) δ 3.77 (t, J=6.2 Hz, 2H), 3.70-3.62 (m, 14H), 3.40 (dd, J=5.6, 4.5 Hz, 2H), 2.64 (t, J=6.2 Hz, 2H). HRMS calcd for C₁₁H₂₁N₃O₆Na [M+Na]+ 314.1328 found 314.1332.

Synthesis of S9. S8 (6.89 mg, 0.024 mmol) and HATU (8.09 mg, 0.021 mmol) were dissolved with DMF (300 μL). The solution was then added with DIPEA (10.32 μL, 0.059 mmol). The mixture was stirred at r.t. for 10 min. The solution was then transferred to a small dry vial containing MMAF TFA salt (10 mg, 0.012 mmol, Levena Biopharma). The reaction was stirred at r.t. for 16 h and then purified by preparative HPLC (monitored at 210 nm). The fractions containing the product were combined and evaporated under reduced pressure to remove organic solvents. The remaining aqueous solution was lyophilized. The product was obtained as clear colorless film coating the bottom of the vial (6.7 mg, 56%). ¹H-NMR (400 MHZ, Methanol-d4) δ 8.33 (dd, J=8.7, 4.8 Hz, 0.42H), 8.16 (dt, J=16.9, 8.7 Hz, 0.52H), 7.92 (d, J=8.2 Hz, 0.31H), 7.83 (dd, J=14.0, 8.6 Hz, 0.43H), 7.33-7.14 (m, 5.00H), 4.86-4.78 (m, 1.02H, overlap with residual water), 4.78-4.53 (m, 2.97H), 4.21-3.91 (m, 2.02H), 3.85-3.73 (m, 2.35H), 3.71-3.57 (m, 16.00H), 3.40-3.33 (m, 6.06H), 3.30-3.26 (m, 3.03H), 3.26-3.05 (m, 6.04H), 3.00-2.87 (m, 1.97H), 2.78-2.63 (m, 2.06H), 2.59-2.42 (m, 2.00H), 2.36-2.18 (m, 2.13H), 2.13-1.94 (m, 2.02H), 1.94-1.69 (m, 2.99H), 1.57 (ddt, J=31.2, 12.6, 7.0 Hz, 0.89H), 1.48-1.23 (m, 3.10H), 1.20 (d, J=6.7 Hz, 1.06H), 1.14 (d, J=6.8 Hz, 0.89H), 1.10-0.81 (m, 21.13H). HRMS calcd for C₅₀H₈₅N₈O₁₃ [M+H]+ 1005.6236 found 1005.6251.

Synthesis of 4. S9 (4.2 mg, 4.18 μmol) was mixed with a solution of S4 (2.45 mg, 8.36 μmol) in methanol (187 μL). Then aqueous solutions of 50 mM THPTA (20.9 μL, 1.045 μmol), 50 mM copper (II) sulfate (20.9 μL, 1.045 μmol), and 100 mM sodium ascorbate (20.9 μL, 2.089 μmol) were added. LC-MS of the crude reaction showed complete consumption of alkyne starting material at 2 h. The reaction was filtered through a 0.2-μm PFTE filter and purified by preparative HPLC (monitored at 210 nm). The fractions containing the product were combined and evaporated to remove organic solvents. The remaining aqueous solution was lyophilized to give the product as slightly yellow film coating the bottom of the vial (2.5 mg, 46.1%). ¹H-NMR (600 MHZ, Methanol-d4) δ 8.29 (d, J=8.2 Hz, 0.26H), 8.19-8.09 (m, 0.38H), 8.03 (s, 0.95H), 7.86 (d, J=8.4 z Hz, 0.30H), 7.79 (d, J=8.7 Hz, 0.39H), 7.29-7.21 (m, 4.11H), 7.20-7.14 (m, 1.44H), 4.72 (q, J=8.9 Hz, 1.09H), 4.67-4.53 (m, 3.16H), 4.10-4.02 (m, 1.05iH), 3.89-3.83 (m, 2.41H), 3.81-3.72 (m, 2.32H), 3.70-3.47 (m, 14.84H), 3.43-3.33 (m, 6.11H), 3.29 (s, 1.93H), 3.24-3.16 (m, 2.85H), 3.14-3.03 (m, 3.66H), 2.98-2.88 (m, 1.96H), 2.87-2.77 (m, 0.70H), 2.75-2.60 (m, 1.86H), 2.50-2.41 (m, 1.83H), 2.33-2.01 (m, 3.09H), 1.95-1.71 (m, 3.07H), 1.66-1.35 (m, 2.85H), 1.29 (s, 1.81H), 1.20 (d, J=6.7 Hz, 1.63H), 1.15 (d, J=6.8 Hz, 1.30H), 1.08-0.76 (m, 20.84H). LC-MS calcd for C₅₇H₈₈N₉O₁₅Br₂ [M+H]+ 1299.19 found 1299.57.

### Example 9: Orthogonal Conjugation of TVD-Fab (Cys_Lys)

### Methods

Cloning expression and purification of TVD-Fab (Cys_Lys). TVD-Fab (Lys_Lys) (Hwang et al., Biomolecules 10, 764, 2020) was used as template for a PCR reaction to mutate Lys at position 99 (Kabat numbering 93) of the upper inner V_{H} to Cys, and DNA encoding TVD-Fab (Cys_Lys) was cloned into mammalian expression vector pCEP4. Heavy and light chain encoding pCEP4 plasmids were co-transfected into Expi293F cells, which had been grown in 300 mL Expi293 Expression Medium at a density of 3×10⁶ cells/mL. Following 5 days of cell culture at 37° C. and 5% CO₂, the supernatant was collected and purified by affinity chromatography using a 1-mL HiTrap KappaSelect column.

Conjugation of TVD-Fab (Cys_Lys). 10 μM TVD-Fab (Cys_Lys) was incubated with 50 μM (5 eq) dibromomaleimide-TAMRA or β-lactam-TAMRA in PBS (pH 8.5) for 1 h at room temperature. 10 μg of each conjugation mixture was loaded onto a 10-well NuPAGE 4-12% Bis-Tris Protein Gel. Fluorescent bands were visualized by blue light on an E-gel Imager (Thermo Fisher) and the gel was subsequently stained by PageBlue Protein Staining Solution.

Catalytic assay. 10 μM TVD-Fab (Cys_Lys) was incubated with 50 μM (5 eq) of β-lactam-TAMRA in PBS (pH 7,4) for 4 h at room temperature. The reactivity of TVD-Fab (Cys_Lys) before and after conjugation to β-lactam-TAMRA was analyzed using methodol as described in Nanna and Rader, Methods Mol. Biol. 2033, 39-52, 2019.

### Results

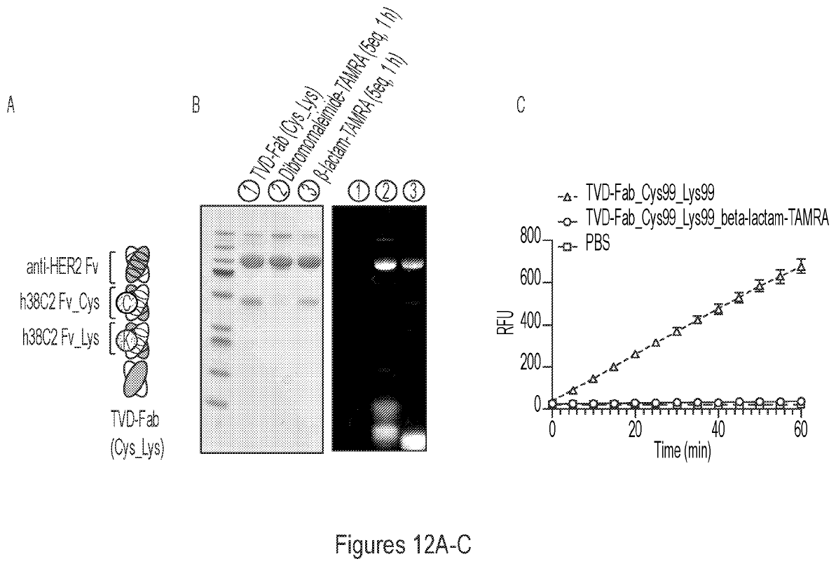

A TVD-Fab targeting HER2 with the outer Fv and carrying the K99C mutation in the upper inner Fv and the parental K99 in the lower inner Fv (FIG. 12A) was generated with high purity. It revealed strong reactivity with dibromomaleimide and β-lactam derivatives, respectively, of the fluorophore TAMRA (FIG. 12B). As expected, incubation with β-lactam-TAMRA resulted in a complete loss of catalytic activity, indicating conjugation to the reactive lysine residue in the lower inner Fv (FIG. 12C).

### REFERENCES

(1) Tong, J. T. W.; Harris, P. W. R.; Brimble, M. A.; Kavianinia, I. An insight into FDA approved antibody-drug conjugates for cancer therapy. Molecules 2021, 26 (19). DOI: ARTN 584710.3390/molecules26195847.
(2) Beck, A.; Goetsch, L.; Dumontet, C.; Corvaia, N. Strategies and challenges for the next generation of antibody drug conjugates. Nat Rev Drug Discov 2017, 16 (5), 315-337. DOI: 10.1038/nrd.2016.268.
(3) Spicer, C. D.; Davis, B. G. Selective chemical protein modification. Nat Commun 2014, 5. DOI: ARTN 474010.1038/ncomms5740.
(4) Guo, J. X.; Kumar, S.; Chipley, M.; Marcq, O.; Gupta, D.; Jin, Z. W.; Tomar, D. S.; Swabowski, C.; Smith, J.; Starkey, J. A.; et al. Characterization and higher-order structure assessment of an interchain cysteine-based ADC: impact of drug loading and distribution on the mechanism of aggregation. Bioconjug Chem 2016, 27 (3), 604-615. DOI: 10.1021/acs.bioconjchem.5b00603.
(5) Behrens, C. R.; Ha, E. H.; Chinn, L. L.; Bowers, S.; Probst, G.; Fitch-Bruhns, M.; Monteon, J.; Valdiosera, A.; Bermudez, A.; Liao-Chan, S.; et al. Antibody-Drug Conjugates (ADCs) Derived from Interchain Cysteine cross-linking demonstrate improved homogeneity and other pharmacological properties over conventional heterogeneous ADCs. Mol Pharmaceut 2015, 12 (11), 3986-3998. DOI: 10.1021/acs.molpharmaceut.5b00432.
(6) Ponte, J. F.; Sun, X. X.; Yoder, N. C.; Fishkin, N.; Laleau, R.; Coccia, J.; Lanieri, L.; Bogalhas, M.; Wang, L. T.; Wilhelm, S.; et al. Understanding how the stability of the thiol-maleimide linkage impacts the pharmacokinetics of lysine-linked antibody-maytansinoid conjugates. Bioconjug Chem 2016, 27 (7), 1588-1598. DOI: 10.1021/acs.bioconjchem.6b00117.
(7) Lyon, R. P.; Setter, J. R.; Bovee, T. D.; Doronina, S. O.; Hunter, J. H.; Anderson, M. E.; Balasubramanian, C. L.; Duniho, S. M.; Leiske, C. I.; Li, F.; et al. Self-hydrolyzing maleimides improve the stability and pharmacological properties of antibody-drug conjugates. Nat Biotechnol 2014, 32 (10), 1059-+. DOI: 10.1038/nbt.2968.
(8) Rader, C.; Turner, J. M.; Heine, A.; Shabat, D.; Sinha, S. C.; Wilson, I. A.; Lerner, R. A.; Barbas, C. F., III A humanized aldolase antibody for selective chemotherapy and adaptor immunotherapy. J Mol Biol 2003, 332 (4), 889-899. DOI: 10.1016/S0022-2836 (03) 00992-6.
(9) Zhu, X. Y.; Tanaka, F.; Lerner, R. A.; Barbas, C. F., III; Wilson, I. A. Direct observation of an enamine intermediate in amine catalysis. J Am Chem Soc 2009, 131 (51), 18206-+. DOI: 10.1021/ja907271a.
(10) Rader, C.; Sinha, S. C.; Popkov, M.; Lerner, R. A.; Barbas, C. F., III Chemically programmed monoclonal antibodies for cancer therapy: adaptor immunotherapy based on a covalent antibody catalyst. Proc Natl Acad Sci USA 2003, 100 (9), 5396-5400. DOI: 10.1073/pnas.0931308100.
(11) Gavrilyuk, J. I.; Wuellner, U.; Barbas, C. F., III beta-Lactam-based approach for the chemical programming of aldolase antibody 38C2. Bioorg Med Chem Lett 2009, 19 (5), 1421-1424. DOI: 10.1016/j.bmcl.2009.01.028.
(12) Hwang, D.; Tsuji, K.; Park, H.; Burke, T. R., Jr.; Rader, C. Site-specific lysine arylation as an alternative bioconjugation strategy for chemically programmed antibodies and antibody-drug conjugates. Bioconjug Chem 2019, 30 (11), 2889-2896. DOI: 10.1021/acs.bioconjchem.9b00609.
(13) Nanna, A. R.; Li, X.; Walseng, E.; Pedzisa, L.; Goydel, R. S.; Hymel, D.; Burke, T. R., Jr.; Roush, W. R.; Rader, C. Harnessing a catalytic lysine residue for the one-step preparation of homogeneous antibody-drug conjugates. Nat Commun 2017, 8 (1), 1112. DOI: 10.1038/s41467-017-01257-1.
(14) Hwang, D.; Nilchan, N.; Nanna, A. R.; Li, X.; Cameron, M. D.; Roush, W. R.; Park, H.; Rader, C. Site-selective antibody functionalization via orthogonally reactive arginine and lysine residues. Cell Chem Biol 2019, 26 (9), 1229-1239 e1229. DOI: 10.1016/j.chembiol.2019.05.010.
(15) Nilchan, N.; Alburger, J. M.; Roush, W. R.; Rader, C. An engineered arginine residue of unusual pH-sensitive reactivity facilitates site-selective antibody conjugation. Biochemistry 2021, 60 (14), 1080-1087. DOI: 10.1021/acs.biochem.0c00955.
(16) Smith, M. E. B.; Schumacher, F. F.; Ryan, C. P.; Tedaldi, L. M.; Papaioannou, D.; Waksman, G.; Caddick, S.; Baker, J. R. Protein modification, bioconjugation, and disulfide bridging using bromomaleimides. J Am Chem Soc 2010, 132 (6), 1960-1965. DOI: 10.1021/ja908610s.
(17) Rader, C.; Turner, J. M.; Heine, A.; Shabat, D.; Sinha, S. C.; Wilson, I. A.; Lerner, R. A.; Barbas, C. F. A humanized aldolase antibody for selective chemotherapy and adaptor immunotherapy. J Mol Biol 2003, 332 (4), 889-899. DOI: 10.1016/s0022-2836 (03) 00992-6.
(18) Toda, N.; Asano, S.; Barbas, C. F., 3rd. Rapid, stable, chemoselective labeling of thiols with Julia-Kocienski-like reagents: a serum-stable alternative to maleimide-based protein conjugation. Angew Chem Int Ed Engl 2013, 52 (48), 12592-12596. DOI: 10.1002/anie.201306241.
(19) Li, X.; Nelson, C. G.; Nair, R. R.; Hazlehurst, L.; Moroni, T.; Martinez-Acedo, P.; Nanna, A. R.; Hymel, D.; Burke, T. R., Jr.; Rader, C. Stable and potent selenomab-drug conjugates. Cell Chem Biol 2017, 24 (4), 433-442 e436. DOI: 10.1016/j.chembiol.2017.02.012.
(20) Hwang, D.; Rader, C. Site-specific antibody-drug conjugates in triple variable domain Fab format. Biomolecules 2020, 10 (5). DOI: 10.3390/biom10050764.
(21) Nilchan, N.; Li, X.; Pedzisa, L.; Nanna, A. R.; Roush, W. R.; Rader, C. Dual-mechanistic antibody-drug conjugate via site-specific selenocysteine/cysteine conjugation. Antib Ther 2019, 2 (4), 71-78. DOI: 10.1093/abt/tbz009.
(22) Yamazaki, C. M.; Yamaguchi, A.; Anami, Y.; Xiong, W.; Otani, Y.; Lee, J.; Ueno, N. T.; Zhang, N.; An, Z.; Tsuchikama, K. Antibody-drug conjugates with dual payloads for combating breast tumor heterogeneity and drug resistance. Nat Commun 2021, 12 (1), 3528. DOI: 10.1038/s41467-021-23793-7.
(23) Nanna, A. R.; Rader, C. Engineering dual variable domains for the generation of site-specific antibody-drug conjugates. Methods Mol Biol 2019, 2033, 39-52. DOI: 10.1007/978-1-4939-9654-4_4.

The invention thus has been disclosed broadly and illustrated in reference to representative embodiments described above. It is understood that various modifications can be made to the present invention without departing from the spirit and scope thereof. It is further noted that all publications, patents and patent applications cited herein are hereby expressly incorporated by reference in their entirety and for all purposes as if each is individually so denoted. Definitions that are contained in text incorporated by reference are excluded to the extent that they contradict definitions in this disclosure.

What is claimed is:

1. An antibody compound comprising a binding site comprising a heavy chain variable region comprising CDRs of SEQ ID NO:1 and a light chain variable region comprising CDRs of SEQ ID NO:2, wherein position 93 of the heavy chain variable region by Kabat numbering is occupied by cysteine.

2. The antibody compound of claim 1, which is humanized.

3. The antibody compound of claim 1, wherein the heavy chain and light chain variable regions comprise SEQ ID NOs: 1 and 2 respectively.

4. The antibody compound of claim 1, which is a dual variable domain (DVD) compound comprising (i) the binding site, and (ii) a second binding site comprising a heavy chain variable region and a light chain variable region recognizing a target of interest.

5. The antibody compound of claim 4, wherein heavy and light chain variable regions of the second binding site are linked to N-termini of the heavy and light chain variable regions of the binding site.

6. The antibody compound of claim 4, which is a homodimeric molecule comprising two antibody arms, each comprising the binding site and the second binding site.

7. The antibody compound of claim 4, which is a heterodimeric molecule comprising two arms, one arm comprising the binding site and the second binding site, the other arm comprising the binding site with lysine instead of cysteine at position 93 of the heavy chain variable region by Kabat numbering and the second binding site.

8. The antibody compound of claim 4, which is a heterodimeric molecule comprising two arms, one arm comprising the binding site and the second binding site, the other arm comprising the binding site with arginine instead of cysteine at position 93 of the heavy chain variable region by Kabat numbering and the second binding site.

9. The antibody compound of claim 4, which is a heterodimeric molecule comprising two arms, one arm comprising the binding site and the binding site with arginine instead of cysteine at position 93 of the heavy chain variable region by Kabat numbering, the other arm comprising the binding site with lysine instead of cysteine at position 93 of the heavy chain variable region by Kabat numbering and the second binding site.

10. The antibody compound of claim 4, which is a heterodimeric molecule comprising two arms, one arm comprising the binding site and the binding site with lysine instead of cysteine at position 93 of the heavy chain variable region by Kabat numbering, the other arm comprising the binding site with arginine instead of cysteine at position 93 of the heavy chain variable region by Kabat numbering and the second binding site.

11. The antibody compound of claim 4, which is a heterodimeric molecule comprising two arms, one arm comprising the binding site with lysine instead of cysteine at position 93 of the heavy chain variable region by Kabat numbering and the binding site with arginine instead of cysteine at position 93 of the heavy chain variable region by Kabat numbering, the other arm comprising the binding site and the second binding site.

12. The antibody compound of claim 1, which is a triple variable domain (TVD) compound comprising (i) the binding site, (ii) the binding site, the binding site with arginine instead of cysteine at position 93 of the heavy chain variable region by Kabat numbering, or the binding site with lysine instead of cysteine at position 93 of the heavy chain variable region by Kabat numbering and (iii) a second binding site comprising a heavy chain variable region and a light chain variable region recognizing a target of interest.

13. The antibody compound of claim 12, wherein heavy and light chain variable regions of the second binding site are linked to N-termini of the heavy and light chain variable regions of the binding site.

14. The antibody compound of claim 12, which is a heterodimeric molecule comprising two arms, one arm comprising the binding site, the binding site with arginine instead of cysteine at position 93 of the heavy chain variable region by Kabat numbering, and the second binding site, the other arm comprising the binding site, the binding site with lysine instead of cysteine at position 93 of the heavy chain variable region by Kabat numbering, and the second binding site.

15. The antibody compound of claim 4, wherein the dual variable domain compound is a bispecific immunoglobulin molecule.

16. The antibody compound of claim 4, wherein the binding site is a Fab, Fab′, F(ab′)₂, Fv or scFv.

17. The antibody compound of claim 12, wherein the target of interest is different than the target recognized by the binding site, wherein the triple variable domain compound is a bispecific immunoglobulin molecule.

18. The antibody compound of claim 12, wherein the binding site is a Fab, Fab′, F(ab′)₂, Fv or scFv.

19. The antibody compound of claim 16 or 18, wherein the binding site is a Fab.

20. The antibody compound of claim 4 or 12, wherein the binding site or second binding site or both comprises a humanized immunoglobulin sequence.

21. The antibody compound of claim 4 or 12, where the target of interest is a tumor cell surface antigen.

22. The antibody compound of claim 20, wherein the tumor cell surface antigen is HER2, HER3, HER4, EGFR, EGFRvIII, FOLR1, FCMR (TOSO), CD19, CD22, CD30, CD33, CD123, CD138, CD79B, PSMA, BCMA, CD38, SLAMF7, Siglec-6, Siglec-15, PDL1, CD70, NECTIN4, TROP2, tissue factor, integrin avb3, GD2, ROR1 or ROR2.

23. An antibody drug conjugate (ADC) comprising at least one drug moiety that is conjugated to an antibody compound via a reactive cysteine residue in the antibody compound, wherein the antibody compound comprise a binding site comprising a heavy chain variable region comprising CDRs of SEQ ID NO:1 and a light chain variable region comprising CDRs of SEQ ID NO:2, wherein position 93 of the heavy chain variable region by Kabat numbering is occupied by cysteine.

24. The antibody drug conjugate of claim 23, wherein the antibody compound is humanized.

25. The antibody drug conjugate of claim 23, wherein the antibody compound is a dual variable domain (DVD) compound comprising (i) the binding site, and (ii) a second binding site comprising a heavy chain variable region and a light chain variable region recognizing a target of interest.

26. The antibody drug conjugate of claim 25, wherein the DVD compound is a homodimeric molecule comprising two antibody arms, each comprising the binding site and the second binding site.

27. The antibody drug conjugate of claim 25, wherein the DVD compound is a heterodimeric molecule comprising two arms, one arm comprising the binding site and the second binding site, the other arm comprising the binding site with lysine instead of cysteine at position 93 of the heavy chain variable region by Kabat numbering and the second binding site.

28. The antibody drug conjugate of claim 25, wherein the DVD compound is a heterodimeric molecule comprising two arms, one arm comprising the binding site and the second binding site, the other arm comprising the binding site with arginine instead of cysteine at position 93 of the heavy chain variable region by Kabat numbering and the second binding site.

29. The antibody drug conjugate of claim 25, wherein the DVD compound is a heterodimeric molecule comprising two arms, one arm comprising the binding site and the binding site with arginine instead of cysteine at position 93 of the heavy chain variable region by Kabat numbering, the other arm comprising the binding site with lysine instead of cysteine at position 93 of the heavy chain variable region by Kabat numbering and the second binding site.

30. The antibody drug conjugate of claim 25, wherein the DVD compound is a heterodimeric molecule comprising two arms, one arm comprising the binding site and the binding site with lysine instead of cysteine at position 93 of the heavy chain variable region by Kabat numbering, the other arm comprising the binding site with arginine instead of cysteine at position 93 of the heavy chain variable region by Kabat numbering and the second binding site.

31. The antibody drug conjugate of claim 25 wherein the DVD compound is a heterodimeric molecule comprising two arms, one arm comprising the binding site with lysine instead of cysteine at position 93 of the heavy chain variable region by Kabat numbering and the binding site with arginine instead of cysteine at position 93 of the heavy chain variable region by Kabat numbering, the other arm comprising the binding site and the second binding site.

32. The antibody drug conjugate of claim 23, wherein the antibody compound is a triple variable domain (TVD) compound comprising (i) the binding site, (ii) the binding site, the binding site with arginine instead of cysteine at position 93 of the heavy chain variable region by Kabat numbering, or the binding site with lysine instead of cysteine at position 93 of the heavy chain variable region by Kabat numbering and (iii) a second binding site comprising a heavy chain variable region and a light chain variable region recognizing a target of interest.

33. The antibody drug conjugate of claim 32 which is a heterodimeric molecule comprising two arms, one arm comprising the binding site, the binding site with arginine instead of cysteine at position 93 of the heavy chain variable region by Kabat numbering, and the second binding site, the other arm comprising the binding site, the binding site with lysine instead of cysteine at position 93 of the heavy chain variable region by Kabat numbering, and the second binding site.

34. The antibody drug conjugate of claim 23, wherein the drug moiety is conjugated to the antibody compound via a linker moiety.

35. The antibody drug conjugate of claim 34, wherein the drug moiety is derivatized with the linker moiety prior to conjugation with the antibody compound.

36. The antibody drug conjugate of claim 34, wherein the linker moiety is a cleavable linker.

37. The antibody drug conjugate of claim 34, wherein the linker moiety comprises maleimide, monobromomaleimide, or dibromomaleimide.

38. The antibody drug conjugate of claim 25, wherein the antibody compound comprises an antigen/hapten-binding fragment of a dual variable domain (DVD) compound that is a Fab, Fab′, F(ab′)₂, Fv or scFv.

39. The antibody drug conjugate of claim 38, wherein the antibody compound comprises a Fab.

40. The antibody drug conjugate of claim 32, wherein the antibody compound comprises an antigen/hapten-binding fragment of a triple variable domain (TVD) compound that is a Fab, Fab′, F(ab′)₂, Fv or scFv.

41. The antibody drug conjugate of claim 40, wherein the antibody compound comprises a Fab.

42. The antibody drug conjugate of claim 25 or 32, where the target of interest is a tumor cell surface antigen.

43. The antibody drug conjugate of claim 42, wherein the tumor cell surface antigen is HER2, HER3, HER4, EGFR, EGFRvIII, FOLR1, FCMR (TOSO), CD19, CD22, CD30, CD33, CD123, CD138, CD79B, PSMA, BCMA, CD38, SLAMF7, Siglec-6, Siglec-15, PDL1, CD70, NECTIN4, TROP2, tissue factor, integrin avb3, GD2, ROR1 or ROR2.

44. The antibody drug conjugate of claim 23, wherein the drug moiety is a cytotoxic agent, an siRNA, or a small molecule-based proteolysis targeting chimera.

45. The antibody drug conjugate of claim 44, wherein the cytotoxic agent is selected from a toxin, a chemotherapeutic agent, a photoabsorber, an antibiotic, a radioactive isotope, a chelated radioactive isotope and a nucleolytic enzyme.

46. The antibody drug conjugate of claim 25, wherein the binding site comprises heavy chain and light chain variable domain sequences respectively shown in SEQ ID NOs: 1 and 2, and the target of interest is HER2.

47. The antibody drug conjugate of claim 46, wherein the drug moiety is an auristatin, a dolostatin, a cemadotin, a camptothecin, an amanitin, a maytansinoid, a pyrrolobenzodiazepine, an indolinobenzodiazepine, a duocarmycin, an endiyne, a doxorubicin, a cepafungin or a Fleximer.

48. The antibody drug conjugate of claim 46, wherein the drug moiety is monomethyl auristatin F (MMAF).

49. The antibody drug conjugate of claim 46, wherein the antibody compound is a DVD-Fab comprising heavy chain and light chain sequences shown in SEQ ID NOs: 8 and 10, respectively.

50. The antibody drug conjugate of claim 46, wherein the antibody compound is a DVD-IgG1 comprising heavy chain and light chain sequences shown in SEQ ID NOs: 9 and 10, respectively.

51. The antibody drug conjugate of claim 50, wherein the DVD-IgG1 is a homodimeric molecule comprising two antibody arms, each comprising heavy chain and light chain sequences shown in SEQ ID NOs: 9 and 10, respectively.

52. The antibody drug conjugate of claim 50, wherein the DVD-IgG1 is a heterodimeric molecule comprising two arms, one arm comprising heavy chain and light chain sequences shown in SEQ ID NOs: 9 and 10, respectively, the other arm comprising heavy chain and light chain sequences shown in SEQ ID NOs: 14 and 10, respectively.

53. The antibody drug conjugate of claim 50, wherein the DVD-IgG1 is a heterodimeric molecule comprising two arms, one arm comprising heavy chain and light chain sequences shown in SEQ ID NOs: 9 and 10, respectively, the other arm comprising heavy chain and light chain sequences shown in SEQ ID NOs: 12 and 10, respectively.

54. The antibody drug conjugate of claim 52 or 53 wherein two different drug moieties are conjugated to the two antibody arms of the heterodimeric DVD-IgG1 molecule.

55. The antibody drug conjugate of claim 46, wherein the antibody compound is a TVD-Fab comprising a heavy chain as shown in any of SEQ ID NOs: 24, 26, or 28 and a light chain sequence as shown in SEQ ID NO:30.

56. The antibody drug conjugate of claim 46, wherein the antibody compound is a TVD-IgG1 comprising a heavy chain as shown in any of SEQ ID NOs: 25, 27, or 29 and a light chain sequence as shown in SEQ ID NO:30.

57. The antibody drug conjugate of claim 56, wherein the TVD-IgG1 is a homodimeric molecule comprising two antibody arms, each comprising a heavy chain as shown in SEQ ID NO:25 and a light chain sequence as shown in SEQ ID NO:30.

58. The antibody drug conjugate of claim 56, wherein the TVD-IgG1 is a homodimeric molecule comprising two antibody arms, each comprising a heavy chain as shown in SEQ ID NO:27 and a light chain sequence as shown in SEQ ID NO:30.

59. The antibody drug conjugate of claim 56, wherein the TVD-IgG1 is a homodimeric molecule comprising two antibody arms, each comprising a heavy chain as shown in SEQ ID NO:29 and a light chain sequence as shown in SEQ ID NO:30.

60. The antibody drug conjugate of claim 56, wherein the TVD-IgG1 is a heterodimeric molecule comprising two arms, one arm comprising heavy chain and light chain sequences shown in SEQ ID NOs: 25 and 30, respectively, the other arm comprising heavy chain and light chain sequences shown in SEQ ID NOs: 27 and 30, respectively.

61. The antibody drug conjugate of claim 56, wherein the TVD-IgG1 is a heterodimeric molecule comprising two arms, one arm comprising heavy chain and light chain sequences shown in SEQ ID NOs: 25 and 30, respectively, the other arm comprising heavy chain and light chain sequences shown in SEQ ID NOs: 29 and 30, respectively.

62. The antibody drug conjugate of claim 56, wherein the TVD-IgG1 is a heterodimeric molecule comprising two arms, one arm comprising heavy chain and light chain sequences shown in SEQ ID NOs: 27 and 30, respectively, the other arm comprising heavy chain and light chain sequences shown in SEQ ID NOs: 29 and 30, respectively.

63. The antibody drug conjugate of any one of claims 58-61 wherein two different drug moieties are conjugated to the two antibody arms of the heterodimeric TVD-IgG1 molecule.

64. The antibody drug conjugate of claim 62 wherein three different drug moieties are conjugated to the antibody arms of the heterodimeric TVD-IgG1 molecule.

65. The antibody drug conjugate of any one of claims 29-31, wherein a first drug moiety is conjugated to the binding site, a second drug moiety is conjugated to the binding site with arginine instead of cysteine at position 93 of the heavy chain variable region by Kabat numbering, and a third drug moiety is conjugated to the binding site with lysine instead of cysteine at position 93 of the heavy chain variable region by Kabat numbering.

66. The antibody drug conjugate of claim 33, wherein a first drug moiety is conjugated to the binding site, a second drug moiety is conjugated to the binding site with arginine instead of cysteine at position 93 of the heavy chain variable region by Kabat numbering, and a third drug moiety is conjugated to the binding site with lysine instead of cysteine at position 93 of the heavy chain variable region by Kabat numbering, wherein the first, second, and third drug moieties are different from each other.

67. The antibody drug conjugate of claim 32, wherein a first drug moiety is conjugated to the binding site and a second drug moiety is conjugated to the binding site with arginine instead of cysteine at position 93 of the heavy chain variable region by Kabat numbering, or the binding site with lysine instead of cysteine at position 93 of the heavy chain variable region by Kabat numbering, wherein the first and second drug moieties are different from each other.

68. A pharmaceutical composition, comprising an effective amount of the antibody drug conjugate of claim 23 and optionally a pharmaceutically acceptable carrier.

69. A method for treating cancer in a subject, comprising administering to the subject in need of treatment the pharmaceutical composition of claim 68.
